(19) United States
(12) Patent Application Publication
(10) Pub. No.: US 2024/0328046 A1
(43) Pub. Date: Oct. 3, 2024

(54) BONDED NONWOVEN FABRIC

(71) Applicants: Fitesa Germany GmbH, Peine (DE); Fitesa Simpsonville, Inc., Simpsonville, SC (US); FitesaCNC Co., Ltd., Rayong (TH)

(72) Inventors: Jessica Teague DAHLE, Greenville, SC (US); Alfredo IZZO, Peine (DE); Nattarin Pitilertsrikul, Rayong (TH); Rene RUSCHEL, Greer, SC (US); Jirawan JIRAPONG, Rayong (TH)

(21) Appl. No.: 18/421,007

(22) Filed: Jan. 24, 2024

Related U.S. Application Data
(60) Provisional application No. 63/454,941, filed on Mar. 27, 2023.

(51) Int. Cl. D04H 1/56; A61F 13/15; A61F 13/515; D04H 1/4291; D04H 1/559

(52) U.S. Cl. CPC D04H 1/56; A61F 13/15764; A61F 13/515; D04H 1/4291; D04H 1/559; A61F 2013/15292; A61F 2013/15365; A61F 2013/15886; A61F 2013/15959; D10B 2321/022; D10B 2401/063; D10B 2509/026

(57) ABSTRACT

A nonwoven fabric having a plurality of fibers bonded with a bond pattern on a surface thereof to form a coherent web, the nonwoven fabric having a vertical axis extending in a machine direction and a horizontal axis extending in a cross direction, the bond pattern having a plurality of spaced apart pairs of arrays extending in the machine, cross, and diagonal directions of the nonwoven fabric, wherein each array includes a plurality of spaced apart bond points having an oblong shape, and wherein the nonwoven fabric has a percent bonded surface area less than about 12% and an average bond point packing value from about 6.5 to 8.0 mm⁻¹. Also provided are a thermal calender, a system for preparing a nonwoven fabric, and associated method.

### CROSS-REFERENCE TO RELATED APPLICATION

The present application claims the benefit of U.S. Application No. 63/454,941, filed Mar. 27, 2023, the contents of which are hereby incorporated by reference in its entirety.

### FIELD

The presently-disclosed invention relates generally to nonwoven fabrics, and more particularly to bonded nonwoven fabrics exhibiting improvements in abrasion resistance and softness.

### BACKGROUND

Nonwoven fabrics are used in a variety of applications such as garments, disposable medical products, and absorbent articles such as diapers and personal hygiene products, among others. New products being developed for these applications have demanding performance requirements, including comfort, conformability to the body, freedom of body movement, good softness and drape, adequate tensile strength and durability, and resistance to surface abrasion, pilling or fuzzing. Accordingly, the nonwoven fabrics which are used in these types of products must be engineered to meet these performance requirements.

Despite significant efforts in developing nonwoven fabrics, there is still a need for products exhibiting improvements in abrasion resistance and mechanical properties without sacrificing other beneficial properties such as softness.

### SUMMARY

One or more embodiments of the invention may provide a nonwoven fabric having desirable properties with respect to abrasion resistance and softness while maintaining good mechanical properties, such as tensile strength and elongation.

Certain embodiments are directed to a nonwoven fabric comprising a plurality of fibers bonded with a bond pattern on a surface thereof to form a coherent web in which the nonwoven fabric has a vertical axis extending in a machine direction and a horizontal axis extending in a cross direction. The bond pattern comprises a plurality of spaced apart pairs of arrays extending in the machine, cross, and diagonal directions of the nonwoven fabric, wherein each array comprises a plurality of spaced apart bond points having an oblong shape, and wherein the nonwoven fabric has a percent bonded surface area less than about 12% and an average bond point packing value greater than 3.5 mm⁻¹, such as from about 6.5 to 8.0 mm⁻¹.

In certain embodiments of the nonwoven fabric, the bond points have an average surface area from about 0.15 to 0.25 mm².

In certain embodiments of the nonwoven fabric, the nonwoven fabric has a bond point density from about 50 to 60 individual bond points per square centimeter.

In certain embodiments of the nonwoven fabric, nonwoven fabric has a Martindale Abrasion Score from about 1.0 to 1.5, a cross direction Handle-o-Meter from about 6.6 to 7.2 grams, a machine direction Handle-O-Meter from about 3.5 to 3.95 grams, and an average abrasion resistance as determined by the weight of removed material from 3.2 to 5.5 grams.

In certain embodiments of the nonwoven fabric, a percent bonded surface area of the nonwoven fabric is from about 9.8 to 10%, the average bond point surface area is from about 0.16 to 0.2 mm², the average bond point packing value is from about 6.75 to 7.25 mm⁻¹, and a bond point density of the nonwoven fabric is from about 52 to 58 individual bond points per square centimeter.

In certain embodiments of the nonwoven fabric, the nonwoven fabric exhibits a Martindale Abrasion Score of 1 to 2, a cross direction Handle-o-Meter from about 6.7 to 7.0 grams, a machine direction Handle-O-Meter from about 3.6 to 3.9 grams, and an average abrasion resistance as determined by the weight of removed material from 3.4 to 3.6 grams.

In certain embodiments of the nonwoven fabric, the bond pattern comprises alternating first and second arrays of individual bond points extending in the cross direction of the nonwoven fabric, wherein the individual bond points of said first array and second array each have a length and a width, and wherein the lengths of the individual bond points of the first array are aligned in substantially the same direction and at an angle that is from about 43 to 47 degrees relative to the horizontal axis of the nonwoven fabric, and the lengths of the individual bond points of the second array are rotated from about 88 to 92 degrees relative to the alignment of the lengths of the individual bond points of the first array, and the individual bond points of the second array are offset in the cross direction from adjacent individual bond points of the first array.

In certain embodiments of the nonwoven fabric, the number of individual bond points per cm² is from about 45 to 60, and in particular, from about 50 to 58, and more particularly, from about 54 to 56.

In certain embodiments of the nonwoven fabric, the percent bonded area of the nonwoven fabric from about 9 to 10.5%, and in particular, from about 9.8 to 10.2%, and more particularly, from about 9.9 to 10%.

In certain embodiments of the nonwoven fabric, a distance between adjacent bonds in the cross direction is from about 1.4 to 1.6 mm, and in particular, from about 1.45 to 1.55 mm, and more particularly, from about 1.48 to 1.52 mm.

In certain embodiments of the nonwoven fabric, a distance between adjacent bond points in a same array in a diagonal direction of the nonwoven fabric is from about 0.7 to 0.95 mm, and in particular, from about 0.75 to 0.90, and more particularly, from about 0.80 to 0.85.

In certain embodiments of the nonwoven fabric, the bond points have an oval-elliptical shape, rectangular shape, rod shape, or a combination thereof.

In certain embodiments of the nonwoven fabric, the bond pattern further defines a plurality of repeating alternating third and fourth arrays of individual bond points extending in the machine direction of the nonwoven fabric, and wherein the individual bond points of the third array are offset in the machine direction from adjacent individual bond points of the fourth array.

In certain embodiments of the nonwoven fabric, the bond pattern further defines a plurality of alternating fifth and sixth arrays of individual bond points extending in a direction that is diagonal relative to the machine direction of the nonwoven fabric.

In certain embodiments of the nonwoven fabric, adjacent bond points in each of the fifth and sixth arrays are directionally aligned at an angle that is from about 88 to 92 degrees relative to a directional alignment of an adjacent individual bond point in the same array.

In certain embodiments of the nonwoven fabric, three adjacent arrays of individual bonds further define a plurality of bond patterns in both the machine and cross directions having a quincunx like bond pattern in which four individual bond points defining corners of the quincunx like bond pattern share a directional orientation that is substantially the same relative to the cross or machine directions of the nonwoven fabric, and in which an individual bond point defining a center point of the quincunx like bond pattern has a directional orientation that is rotated from about 88 to 92 degrees relative to the directional orientation of the individual bond points defining the corners of the quincunx like bond pattern.

In certain embodiments of the nonwoven fabric, an angle formed by an array extending in the diagonal direction and array extending in the cross direction is from about 43 to 47 degrees, and in particular, about 45 degrees.

In certain embodiments of the nonwoven fabric, the nonwoven fabric exhibits a Martindale Abrasion Score of less than 1.5 and in particular, from 1.2 to 1.5, and more particularly, from about 1.40 to 1.45.

In certain embodiments of the nonwoven fabric, the nonwoven fabric has a basis weight from about 20 to 30 gsm and exhibits a softness as demonstrated by a cross direction handle-o-meter of less than 7.0 grams (g), such as less than 7.9 grams or less than 7.5 grams.

In certain embodiments of the nonwoven fabric, the nonwoven fabric has a basis weight from about 20 to 30 gsm and exhibits a softness as demonstrated by a machine direction handle-o-meter of less than 3.9 grams (g), such as less than 3.8 grams or less than 3.78 grams.

In certain embodiments of the nonwoven fabric, the nonwoven fabric exhibits an increase in tensile strength, percent elongation, abrasion resistance, and softness in comparison to a similarly prepared nonwoven fabric with the exception that the similarly fabric was point bonded with a bonding pattern having a bond point packing of less than 3.5 mm⁻¹.

In certain embodiments of the nonwoven fabric, the nonwoven fabric exhibits a tensile strength that is 10% or more greater in comparison to a similarly prepared nonwoven fabric having a bond point packing of less than 3.5 mm⁻¹, such as an increase in ensile strength that is from 10% to 50%, such as 12 to 30%, 12 to 25%, 12 to 24%, or 12 to 20% greater than the tensile strength of a similarly prepared nonwoven fabric having a bond point packing of less than 3.5 mm⁻¹.

In certain embodiments of the nonwoven fabric, the nonwoven fabric exhibits an increase in tensile strength that is 10% or greater in comparison to a similarly prepared nonwoven fabric having a bonded surface area greater than 12%,

In certain embodiments of the nonwoven fabric, the nonwoven fabric exhibits an increase in tensile strength that is from 10% to 30%, such as 12 to 20%, greater than the tensile strength of a similarly prepared nonwoven fabric having a bonded surface area greater than 12%.

In certain embodiments of the nonwoven fabric, the nonwoven fabric exhibits an increase in machine direction tensile strength that is from about 10 to 50% greater in comparison to a similarly prepared nonwoven fabric having a bond point packing of less than 3.5 mm⁻¹.

In certain embodiments of the nonwoven fabric, the nonwoven fabric exhibits an increase in machine direction tensile strength that is from about 10 to 30%, such as from about 12 to 20%, or from about 12 to 15% in comparison to a similarly prepared nonwoven fabric having a bond point packing of less than 3.5 mm⁻¹.

In certain embodiments of the nonwoven fabric, the nonwoven fabric exhibits an increase in cross direction tensile strength that is from about 10 to 50% greater in comparison to a similarly prepared nonwoven fabric having a bond point packing of less than 3.5 mm⁻¹.

In certain embodiments of the nonwoven fabric, the nonwoven fabric exhibits an increase in cross direction tensile strength that is from about 10 to 30%, such as from about 15 to 25%, from about 18 to 24%, or from about 19 to 21% in comparison to a similarly prepared nonwoven fabric having a bond point packing of less than 3.5 mm⁻¹.

In certain embodiments of the nonwoven fabric, the nonwoven fabric exhibits an increase in machine direction tensile strength that is from about 10 to 50% greater in comparison to a similarly prepared nonwoven fabric having a bonded surface area greater than 12%.

In certain embodiments of the nonwoven fabric, the nonwoven fabric exhibits an increase in machine direction tensile strength that is from about 10 to 30%, such as from about 12 to 20%, or from about 12 to 15% in comparison to a similarly prepared nonwoven fabric having a bonded surface area greater than 12%.

In certain embodiments of the nonwoven fabric, the nonwoven fabric exhibits an increase in cross direction tensile strength that is from about 10 to 50% greater in comparison to a similarly prepared nonwoven fabric having a bonded surface area greater than 12%.

In certain embodiments of the nonwoven fabric, the nonwoven fabric exhibits an increase in cross direction tensile strength that is from about 10 to 30%, such as from about 15 to 25%, from about 18 to 24%, or from about 19 to 21% in comparison to a similarly prepared nonwoven fabric having a bonded surface area greater than 12%.

In certain embodiments of the nonwoven fabric, the nonwoven fabric exhibits an increase in percent elongation that is from about 4 to 50% in comparison to a similarly prepared nonwoven fabric having a bond point packing of less than 3.5 mm⁻¹.

In certain embodiments of the nonwoven fabric, the nonwoven fabric exhibits an increase percent elongation that is from about 4 to 25%, such as from about 5 to 20%, or from about 5 to 15%, in comparison to a similarly prepared nonwoven fabric having a bond point packing of less than 3.5 mm⁻¹.

In certain embodiments of the nonwoven fabric, the nonwoven fabric exhibits an increase in percent elongation that is from about 4 to 50% in comparison to a similarly prepared nonwoven fabric having a bonded surface area greater than 12%.

In certain embodiments of the nonwoven fabric, the nonwoven fabric exhibits an increase in percent elongation that is from about 4 to 25%, such as from about 5 to 20%, or from about 5 to 15%, in comparison to a similarly prepared nonwoven fabric having a bonded surface area greater than 12%.

In certain embodiments of the nonwoven fabric, the nonwoven fabric exhibits an increase in abrasion resistance as exemplified by a percent difference in the Martindale Abrasion Score of 10 to 30% in comparison to a similarly prepared nonwoven fabric having a bond point packing of less than 3.5 mm⁻¹.

In certain embodiments of the nonwoven fabric, the nonwoven fabric exhibits an increase in abrasion resistance as exemplified by a percent difference in the Martindale Abrasion Score of from about 10 to 25%, such as from about 12 to 24%, or from about 1S to 22% A, in comparison to a similarly prepared nonwoven fabric having a bond point packing of less than 3.5 mm⁻¹.

In certain embodiments of the nonwoven fabric, the nonwoven fabric exhibits an increase in abrasion resistance exemplified by a percent difference in the Martindale Abrasion Score of 10 to 30% in comparison to a similarly prepared nonwoven fabric having a bonded surface area greater than 12%.

In certain embodiments of the nonwoven fabric, the nonwoven fabric exhibits an increase in abrasion resistance as exemplified by a percent difference in the Martindale Abrasion Score of from about 10 to 25%, such as from about 12 to 24%., or from about 18 to 22%, in comparison to a similarly prepared nonwoven fabric having a bonded surface area greater than 12%.

In certain embodiments of the nonwoven fabric, the nonwoven fabric exhibits an average percent decrease in the weight of material removed during the abrasion testing (in accordance with Test Method NWSP 20.5) from 5 to 150% in comparison to a similarly prepared nonwoven fabric having a bond point packing of less than 3.5 mm⁻¹.

In certain embodiments of the nonwoven fabric, the nonwoven fabric exhibits an average percent decrease in the weight of material removed during the abrasion testing (in accordance with Test Method NWSP 20.5) from 8 to 120%, such as 9 to 95% in comparison to a similarly prepared nonwoven fabric having a bond point packing of less than 3.5 mm⁻¹.

In certain embodiments of the nonwoven fabric, the nonwoven fabric exhibits an average percent decrease in the weight of material removed during the abrasion testing (in accordance with Test Method NWSP 20.5) from 5 to 150% in comparison to a similarly prepared nonwoven fabric having a bonded surface area greater than 12%.

In certain embodiments of the nonwoven fabric, the nonwoven fabric exhibits an average percent decrease in the weight of material removed during the abrasion testing (in accordance with Test Method NWSP 20.5) from 8 to 120%, such as 9 to 95% in comparison to a similarly prepared nonwoven fabric having a bonded surface area greater than 12%.

In certain embodiments of the nonwoven fabric, the nonwoven fabric exhibits an increase in softness as demonstrated by an average difference of Handle-O-Meter values from about 5 to 20% in comparison to a similarly prepared nonwoven fabric having a bond point packing of less than 3.5 mm⁻¹.

In certain embodiments of the nonwoven fabric, the nonwoven fabric exhibits an increase in softness as demonstrated by an average difference of Handle-O-Meter values from about 6 to 15%, such as 8 to 10% in comparison to a similarly prepared nonwoven fabric having a bond point packing of less than 3.5 mm⁻¹.

In certain embodiments of the nonwoven fabric, the nonwoven fabric exhibits an increase in softness as demonstrated by an average difference of Handle-O-Meter values from about 5 to 20% in comparison to a similarly prepared nonwoven fabric having a bonded surface area greater than 12%.

In certain embodiments of the nonwoven fabric, the nonwoven fabric exhibits an increase in softness as demonstrated by an average difference of Handle-O-Meter values from about 6 to 15%, such as 8 to 10% in comparison to a similarly prepared nonwoven fabric having a bonded surface area greater than 12%.

In certain embodiments of the nonwoven fabric, the nonwoven fabric exhibits an increase in softness as demonstrated by an average difference of Handle-O-Meter values from about 5 to 20% in comparison to a similarly prepared nonwoven fabric having a bond point packing of less than 3.5 mm⁻¹.

In certain embodiments of the nonwoven fabric, the nonwoven fabric has a percent bonded area from about 9.6 to 10.4%, an average individual bond point surface area from about 0.15 to 0.25 mm², an average bond point packing value from about 6.75 to 7.25 mm⁻¹, a bond point density from about 50 to 60 individual bond points per square centimeter, a Martindale Abrasion Score from about 1.0 to 1.5, a cross direction Handle-o-Meter from about 6.6 to 7.2 grams, a machine direction Handle-O-Meter from about 3.5 to 3.95 grams, and an average abrasion resistance as determined by the weight of removed material from 3.2 to 5.5 grams.

In certain embodiments of the nonwoven fabric, the nonwoven fabric has a percent bonded area from about 9.8 to 10%, an average individual bond point surface area from about 0.15 to 0.2 mm², an average bond point packing value from about 7 to 7.2 mm⁻¹, a bond point density from about 52 to 58 individual bond points per square centimeter, a Martindale Abrasion Score from about 1.42 to 1.45, a cross direction Handle-o-Meter from about 6.7 to 7.0 grams, a machine direction Handle-O-Meter from about 3.6 to 3.9 grams, and an average abrasion resistance as determined by the weight of removed material from 3.4 to 3.6 grams.

In certain embodiments of the nonwoven fabric, the nonwoven fabric has a percent bonded area from about 9.6 to 10.2%, an average individual bond surface area from about 0.15 to 0.25 mm², an average bond point packing value from about 6.75 to 7.25 mm⁻¹, and an average increase in tensile strengths that are 10% greater, such as from about 10 to 50%, from about 12 to 24%, or from about 12 to 22%, in comparison to a similarly prepared nonwoven fabric having a bond point packing of less than 3.5 mm⁻¹.

In certain embodiments of the nonwoven fabric, the nonwoven fabric has a percent bonded area from about 9.6 to 10.2%, an average individual bond surface area from about 0.15 to 0.25 mm², an average bond point packing value from about 6.75 to 7.25 mm⁻¹, and an average increase in tensile strengths that are 10% greater, such as from about 10 to 50%, from about 12 to 24%, or from about 12 to 22%, in comparison to a similarly prepared nonwoven fabric having a bonded surface area greater than 12%, such as 18.1%.

In certain embodiments of the nonwoven fabric, the nonwoven fabric has a percent bonded area from about 9.6 to 10.2%, an average individual bond surface area from about 0.15 to 0.25 mm², an average bond point packing value from about 6.75 to 7.25 mm⁻¹, and an average increase in percent elongation that is from about 4 to 50%, such as from about 4 to 20%, from about 4 to 15%, or from about 4 to 14%, in comparison to a similarly prepared nonwoven fabric having a bond point packing of less than 3.5 mm⁻¹.

In certain embodiments of the nonwoven fabric, the nonwoven fabric has a percent bonded area from about 9.6 to 10.2%, an average individual bond surface area from about 0.15 to 0.25 mm², an average bond point packing value from about 6.5 to 7.5 mm⁻¹, and an average increase in percent elongation that is from about 4 to 50%, such as from about 4 to 20%, from about 4 to 15%, or from about 4 to 14%, in comparison to a similarly prepared nonwoven fabric having a bonded surface area greater than 12%, such as 18.1%.

In certain embodiments of the nonwoven fabric, the nonwoven fabric has a percent bonded area from about 9.6 to 10.2%, an average individual bond surface area from about 0.15 to 0.25 mm², an average bond point packing value from about 6.75 to 7.25 mm⁻¹, and one or more of the following properties:
an improvement in abrasion resistance as exemplified by a percent difference in the Martindale Abrasion Score of 10 to 30% in comparison to a similarly prepared nonwoven fabric having a bond point packing of less than 3.5 mm⁻¹;
an average percent decrease in the weight of material removed during the abrasion testing (in accordance with Test Method NWSP 20.5) from 5 to 150%, such as from about 8 to 120% or 9 to 95% in comparison to a similarly prepared nonwoven fabric having a bond point packing of less than 3.5 mm⁻¹; and
improvements in softness as demonstrated by an average improvement of Handle-O-Meter values from about 5 to 20%, such as from about 6 to 15% or 8 to 10%, in comparison to a similarly prepared non woven fabric having a bond point packing of less than 3.5 mm⁻¹.

In certain embodiments of the nonwoven fabric, the nonwoven fabric has a percent bonded area from about 9.6 to 10.2%, an average individual bond surface area from about 0.15 to 0.25 mm², an average bond point packing value from about 6.75 to 7.25 mm⁻¹, and one or more of the following properties:
an improvement in abrasion resistance as exemplified by a percent difference in the Martindale Abrasion Score of 10 to 30% in comparison to a similarly prepared nonwoven fabric having a bonded surface area greater than 12%, such as 18.1%;
an average percent decrease in the weight of material removed during the abrasion testing (in accordance with Test Method NWSP 20.5) from 5 to 150%, such as from about 8 to 120% or 9 to 95% in comparison to a similarly prepared nonwoven fabric having a bonded surface area greater than 12%, such as 18.1%; and
improvements in softness as demonstrated by an average improvement of Handle-O-Meter values from about 5 to 20%, such as from about 6 to 15% or 8 to 10%, in comparison to a similarly prepared nonwoven fabric having a bonded surface area greater than 12%, such as 18.1%.

In certain embodiments of the nonwoven fabric, the nonwoven fabric has a percent bonded area from about 9.6 to 10.2%, an average individual bond surface area from about 0.15 to 0.25 mm², an average bond point packing value from about 6.75 to 7.25 mm⁻¹, and one or more of the following properties:
an improvement in abrasion resistance as exemplified by a percent difference in the Martindale Abrasion Score of 10 to 30% in comparison to a similarly prepared nonwoven fabric having a bond point packing of less than 3.5 mm⁻¹;
an average percent decrease in the weight of material removed during the abrasion testing (in accordance with Test Method NWSP 20.5) from 5 to 150%, such as from about 8 to 120% or 9 to 95% in comparison to a similarly prepared nonwoven fabric having a bond point packing of less than 3.5 mm⁻¹;
improvements in softness as demonstrated by an average improvement of Handle-O-Meter values from about 5 to 20%, such as from about 6 to 15% or 8 to 10%, in comparison to a similarly prepared nonwoven fabric having a bond point packing of less than 3.5 mm⁻¹;
an increase in machine direction (MD) tensile strengths that are from about 10 to 50% greater, such as from about 10 to 30%, such as from about 12 to 20%, or from about 12 to 15% greater, in comparison to a similarly prepared nonwoven fabric having a bond point packing of less than 3.5 mm⁻¹;
an increase in cross direction (CD) tensile strengths that are from about 10 to 50% greater, such as from about 10 to 30%, such as from about 15 to 25%, from about 18 to 24%, or from about 19 to 21%, in comparison to a similarly prepared nonwoven fabric having a bond point packing of less than 3.5 mm⁻¹; and
an increase in percent elongation that is from about 4 to 50%, such as from about 5 to 20%, or from about 5 to 15%, in comparison to a similarly prepared nonwoven fabric having a bond point packing of less than 3.5 mm⁻¹.

In certain embodiments of the nonwoven fabric, the nonwoven fabric has a percent bonded area from about 9.6 to 10.2%, an average individual bond surface area from about 0.15 to 0.25 mm², an average bond point packing value from about 6.75 to 7.25 mm⁻¹, and one or more of the following properties:
an improvement in abrasion resistance as exemplified by a percent difference in the Martindale Abrasion Score of 10 to 30% in comparison to a similarly prepared nonwoven fabric having a bonded surface area greater than 12%, such as 18.1%;
an average percent decrease in the weight of material removed during the abrasion testing (in accordance with Test Method NWSP 20.5) from 5 to 150%, such as from about 8 to 120% or 9 to 95% in comparison to a similarly prepared nonwoven fabric having a bonded surface area greater than 12%, such as 18.1%;
an improvement in softness as demonstrated by an average improvement of Handle-O-Meter values from about 5 to 20%, such as from about 6 to 15% or 8 to 10%, in comparison to a similarly prepared nonwoven fabric having a bonded surface area greater than 12%, such as 18.1%;
an increase in machine direction (MD) tensile strengths that are from about 10 to 50% greater, such as from about 10 to 30%, such as from about 12 to 20%, or from about 12 to 15% greater, in comparison to a similarly prepared nonwoven fabric having a bonded surface area greater than 12%, such as 18.1%;
an increase in cross direction (CD) tensile strengths that are from about 10 to 50% greater, such as from about 10 to 30%, such as from about 15 to 25%, from about 18 to 24%, or from about 19 to 21%, in comparison to a similarly prepared nonwoven fabric having a bonded surface area greater than 12%, such as 18.1%; and
an increase in percent elongation that is from about 4 to 50%, such as from about 5 to 20%, or from about 5 to 15%, in comparison to a similarly prepared nonwoven fabric having a bonded surface area greater than 12%, such as 18.1%.

In certain embodiments of the nonwoven fabric, the nonwoven fabric comprises a spunbond layer.

In certain embodiments of the nonwoven fabric, the nonwoven fabric comprises a first spunbond layer having low or no crimping filaments and a second layer comprising crimped filaments.

In certain embodiments of the nonwoven fabric, the nonwoven fabric comprises at least two layers in which one of the layers is selected from the group of meltblown layer; carded fabric layer, spunbond layer, resin bonded layer, airlaid fabric layer, and a spunlace layer.

In certain embodiments of the nonwoven fabric, the nonwoven fabric is in an absorbent article.

In certain embodiments, embodiments of the invention provide an absorbent article comprising the nonwoven fabric. In certain embodiments, the invention relates to the use of the nonwoven fabric, wherein the nonwoven fabric is in an absorbent article.

In certain embodiments, embodiments of the invention provide a nonwoven article comprising the nonwoven fabric. In certain embodiments of the nonwoven fabric, the nonwoven is a component of a composite sheet material. In certain embodiments, embodiments of the invention provide a composite sheet material comprising the nonwoven fabric. In some embodiments, the nonwoven fabric comprises a composite sheet material. In some embodiments, a sheet material comprises a meltblown layer comprising the nonwoven fabric in accordance with an embodiment of the invention. In certain embodiments, the meltblown layer is sandwiched between two spunbond layers, and in which at least one of the spunbond layers is in accordance with the nonwoven fabric layer of the present disclosure.

Additional aspects are directed a calender bonding unit have an engraved pattern roll configured to impart a pattern in accordance with one or more embodiments of the disclosure.

In certain embodiments, a calender bonding unit for point bonding a sheet material is provided in which the calender bonding unit comprising a pair of cooperating cylindrical rolls in which at least one of the rolls includes an engraved pattern thereon, the engraved pattern comprising a plurality of individual, spaced apart bonding points that extend radially outward from a surface of the roll, the plurality of bonding points defining a pattern comprising a plurality of spaced apart arrays that extend in the cross and radial direction of the roll, and are configured and arranged to thermally point bond a nonwoven fabric in which a percent bonded surface area of the nonwoven fabric is less than about 12% and an average bond point packing value of the nonwoven fabric is from about 6.5 to 8 mm⁻¹.

In certain embodiments, the engraved pattern further comprises a plurality of spaced apart pairs of arrays that extend circumferentially about the roll in a spiral-like shape.

In certain embodiments, the bonding points have a continuous side wall and a raised surface, the raised surfaces have an average surface area from about 0.15 to 0.25 mm².

In certain embodiments, the number of bonding points is from about 50 to 60 individual bonding points per square centimeter.

In certain embodiments, an average length of the bonding points is from about 0.74 to 0.78 mm, and an average width of the bonding points is from about 0.24 to 0.36 mm.

In a further aspect of the disclosure, embodiments are directed to a system for preparing a bonded nonwoven fabric.

In certain embodiments, a system of preparing a nonwoven fabric is provide in which the system includes:
a first polymer source.
a spinbeam in communication with the first polymer source, the spinbeam configured and arranged to produce a plurality of polymer fibers;
a collection surface disposed below the spinbeam for depositing the plurality of polymer fibers thereon to form a web of fibers; and
a thermal bonding unit disposed downstream of the spin beam, the thermal bonding unit comprising a pair of cooperating cylindrical rolls in which at least one of the rolls includes an engraved pattern thereon, the engraved pattern comprising a plurality of individual, spaced apart bonding points that extend radially outward from a surface of the roll, the plurality of bonding points defining a pattern comprising a plurality of spaced apart arrays that extend in the cross and radial direction of the roll, and are configured and arranged to thermally point bond the web of fibers to form a nonwoven fabric in which a percent bonded surface area of the nonwoven fabric is less than about 12% and an average bond point packing value of the nonwoven fabric is from about 6.5 to 8 mm⁻¹.

Further aspects of the invention are also directed to methods of preparing a nonwoven fabric including the step of passing a nonwoven web through a heated calender roll in which the calender roll comprises a pair of cooperating cylindrical rolls in which at least one of the rolls includes an engraved pattern thereon, the engraved pattern comprising a plurality of individual, spaced apart bonding points that extend radially outward from a surface of the roll, the plurality of bonding points defining a pattern comprising a plurality of spaced apart arrays that extend in the cross and radial direction of the roll, and are configured and arranged to thermally point bond the web of fibers to form a nonwoven fabric in which a percent bonded surface area of the nonwoven fabric is less than about 12% and an average bond point packing value of the nonwoven fabric is from about 6.5 to 8 mm⁻¹.

In certain aspects, embodiments of the invention are directed to a nonwoven fabric comprising a plurality of fibers bonded with a bond pattern on a surface thereof to form a coherent web, the nonwoven fabric having a vertical axis extending in a machine direction and a horizontal axis extending in a cross direction, the bond pattern comprising a plurality of spaced apart pairs of arrays extending in the machine, cross, and diagonal directions of the nonwoven fabric, wherein each array comprises a plurality of spaced apart bond points having an oblong shape, and wherein the nonwoven fabric has a percent bonded surface area less than about 14%, a collective average bond distance from about 1.5 to 1.7 mm, and an average bond point packing value from about 3.0 to 5.0 mm⁻¹.

Additional aspects of the invention are directed to an associated method, system, and apparatus for preparing a nonwoven fabric in which the nonwoven fabric has a percent bonded surface area less than about 14%, a collective average bond distance from about 1.5 to 1.7 mm, and an average bond point packing value from about 3.0 to 5.0 mm⁻¹.

In certain embodiments, the fibers of nonwoven fabrics comprise a blend of a polypropylene resin and less than 20%, by weight, of a polypropylene copolymer.

### BRIEF DESCRIPTION OF THE SEVERAL DRAWINGS

Having thus described the invention in general terms, reference will now be made to the accompanying drawings, which are not necessarily drawn to scale, and wherein:

FIG. 1 illustrates a bonded nonwoven fabric having a bond pattern that is in accordance with at least one embodiment of the invention;

FIGS. 2A and 2B illustrate a bond point that is in accordance with one or more embodiments of the present invention;

FIGS. 3A-3C illustrate various bonding arrays of a bond pattern on the surface of a nonwoven fabric in accordance with at least one embodiment of the present invention;

FIG. 4 illustrates a bond pattern on the surface of a nonwoven fabric in accordance with at least one embodiment of the present invention;

FIGS. 5A and 5B illustrate a secondary bond pattern formed on the surface of a nonwoven fabric in accordance with at least one embodiment of the present invention;

FIG. 6 illustrates a bond pattern on the surface of a nonwoven fabric in accordance with at least one embodiment of the present invention;

FIGS. 7A-7D illustrate a further bond pattern on the surface of a nonwoven fabric in accordance with at least one embodiment of the present invention;

FIGS. 8A-8C illustrate a further bond pattern on the surface of a nonwoven fabric in accordance with at least one embodiment of the present invention;

FIG. 9A illustrates a system for preparing a bonded nonwoven fabric in accordance with at least one embodiment of the present invention;

FIG. 9B illustrates a system for preparing a bonded nonwoven fabric in accordance with at least one embodiment of the present invention;

FIG. 10 illustrates a system for preparing a bonded nonwoven fabric in accordance with at least one embodiment of the present invention;

FIG. 11 illustrates a calender bonding unit in accordance with at least one embodiment of the present invention;

FIG. 12 illustrates a patterned roll of the calender bonding unit of FIG. 11;

FIGS. 13A-13C illustrate various views of a bonding point in accordance with at least one embodiment of the present invention; and

FIGS. 14A-14B illustrate multilayer nonwoven fabrics in accordance with at least one embodiment of the present invention.

### DETAILED DESCRIPTION

The invention now will be described more fully hereinafter with reference to the accompanying drawings, in which some, but not all embodiments of the inventions are shown. Indeed, this inventions may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

The terms “first,” “second,” and the like, “primary,” “exemplary,” “secondary,” and the like, do not denote any order, quantity, or importance, but rather are used to distinguish one element from another. Further, the terms “a,” “an,” and “the” do not denote a limitation of quantity, but rather denote the presence of “at least one” of the referenced item.

Each embodiment disclosed herein is contemplated as being applicable to each of the other disclosed embodiments. All combinations and sub-combinations of the various elements described herein are within the scope of the invention.

It is understood that where a parameter range is provided, all integers within that range, and tenths and hundredths thereof, are also provided by the invention. For example, “5-10%” includes 5%, 6%, 7%, 8%, 9%, and 10%; 5.0%, 5.1%, 5.2% . . . 9.8%, 9.9%, and 10.0%; and 5.00%, 5.01%, 5.02% . . . 9.98%, 9.99%, and 10.00%.

As used herein, the terms “about.” “approximately,” and “substantially” in the context of a numerical value or range means±10% of the numerical value or range recited or claimed, and in particular, encompasses values within a standard margin of error of measurement (e.g. SEM) of a stated value or variations ±0.5, 1%, 5%, or 10% from a specified value.

For the purposes of the present application, the following terms shall have the following meanings:

The term “fiber” can refer to a fiber of finite length or a filament of infinite length.

As used herein, the term “monocomponent” refers to fibers formed from one polymer or formed from a single blend of polymers. Of course, this does not exclude fibers to which additives have been added for color, anti-static properties, lubrication, hydrophilicity, liquid repellency, etc.

As used herein, the term “multicomponent” refers to fibers formed from at least two polymers (e.g., bicomponent fibers) that are extruded from separate extruders. The at least two polymers can each independently be the same or different from each other, or be a blend of polymers. The polymers are arranged in substantially constantly positioned distinct zones across the cross-section of the fibers. The components may be arranged in any desired configuration, such as sheath-core, side-by-side, pie, island-in-the-sea, and so forth. Various methods for forming multicomponent fibers are described in U.S. Pat. No. 4,789,592 to Taniguchi et al. and U.S. Pat. No. 5,336,552 to Strack et al., U.S. Pat. No. 5,108,820 to Kaneko, et al., U.S. Pat. No. 4,795,668 to Kruege, et al., U.S. Pat. No. 5,382,400 to Pike, et al., U.S. Pat. No. 5,336,552 to Strack, et al., and U.S. Pat. No. 6,200,669 to Marmon, et al., which are incorporated herein in their entirety by reference. Multicomponent fibers having various irregular shapes may also be formed, such as described in U.S. Pat. No. 5,277,976 to Hogle, et al., U.S. Pat. No. 5,162,074 to Hills, U.S. Pat. No. 5,466,410 to Hills, U.S. Pat. No. 5,069,970 to Largman, et al., and U.S. Pat. No. 5,057,368 to Largman, et al., which are incorporated herein in their entirety by reference.

As used herein the terms “nonwoven,” “nonwoven web” and “nonwoven fabric” refer to a structure or a web of material which has been formed without use of weaving or knitting processes to produce a structure of individual fibers or threads which are intermeshed, but not in an identifiable, repeating manner. Nonwoven webs have been, in the past, formed by a variety of conventional processes such as, for example, meltblown processes, spunbond processes, and staple fiber carding processes.

As used herein, the term “meltblown” refers to a process in which fibers are formed by extruding a molten thermoplastic material through a plurality of fine, usually circular, die capillaries into a high velocity gas (e.g. air) stream which attenuates the molten thermoplastic material and forms fibers, which can be to microfiber diameter. Thereafter, the meltblown fibers are carried by the gas stream and are deposited on a collecting surface to form a web of random meltblown fibers. Such a process is disclosed, for example, in U.S. Pat. No. 3,849,241 to Buntin et al.

As used herein, the term “machine direction” or “MD” refers to the direction of travel of the nonwoven web during manufacturing.

As used herein, the term “cross direction” or “CD” refers to a direction that is perpendicular to the machine direction and extends laterally across the width of the nonwoven web.

As used herein, the term “diagonal direction” or “DD” refers to a direction that is angled from greater than 0° to less than 90° relative to one or more of the cross and machine directions.

As used herein, and unless indicated to the contrary, the term “molecular weight” refers to the weight average molecular weight (Mw), and is expressed in grams/mol. The weight average molecular weight can be determined using commonly known techniques, such as gel permeation chromatography (GPC).

As used herein, the term “spunbond” refers to a process involving extruding a molten thermoplastic material as filaments from a plurality of fine, usually circular, capillaries of a spinneret, with the filaments then being attenuated and drawn mechanically or pneumatically. The filaments are deposited on a collecting surface to form a web of randomly arranged substantially continuous filaments which can thereafter be bonded together to form a coherent nonwoven fabric. The production of spunbond non-woven webs is illustrated in patents such as, for example, U.S. Pat. Nos. 3,338,992; 3,692,613, 3,802,817; 4,405,297 and 5,665,300. In general, these spunbond processes include extruding the filaments from a spinneret, quenching the filaments with a flow of air to hasten the solidification of the molten filaments, attenuating the filaments by applying a draw tension, either by pneumatically entraining the filaments in an air stream or mechanically by wrapping them around mechanical draw rolls, depositing the drawn filaments onto a foraminous collection surface to form a web, and bonding the web of loose filaments into a nonwoven fabric. The bonding can be any thermal or chemical bonding treatment, with thermal point bonding being typical.

As used herein “thermal point bonding” involves passing a material such as one or more webs of fibers to be bonded between a heated calender roll and an anvil roll. The calender roll is typically engraved with a pattern so that the fabric is bonded in discrete point bond sites rather than being bonded across its entire surface.

As used herein, the term “bond density” refers to the number of individual bond points in a given surface area of the nonwoven fabric.

As used herein the term “polymer” generally includes, but is not limited to, homopolymers, copolymers, such as, for example, block, graft, random and alternating copolymers, terpolymers, etc. and blends and modifications thereof. Furthermore, unless otherwise specifically limited, the term “polymer” shall include all possible geometrical configurations of the material, including isotactic, syndiotactic and random symmetries.

### Nonwoven Fabric

In a first aspect, embodiments of the disclosure are directed to thermally point bonded nonwoven fabrics having improved abrasion resistance and softness. Generally, improvements in the abrasion resistance of the surface of a nonwoven fabric may be obtained by increasing the percentage of bonded area of the nonwoven fabric. That is, the greater percentage area of the fibers on the surface of the fabric that are subjected to thermal point bonding, the greater the abrasion resistance of the fabric due to a greater number of fibers being bonded to adjacent fibers. However, such improvements in abrasion resistance typically result in a decrease in softness of the bonded nonwoven fabric. Accordingly, there is generally a recognized trade-off between improvements in abrasion resistance and improvements in softness of the bonded nonwoven fabric.

Advantageously and surprisingly, the inventors of the present disclosure have discovered that improvements in both abrasion resistance and softness may be obtained with nonwoven fabrics that are thermally point bonded with bond patterns in accordance with one or more embodiments of the present invention. In particular, it has been discovered that a bonded nonwoven fabric having a first bond pattern comprising a plurality of alternating arrays composed of individual bond points in both the machine direction and cross direction of the nonwoven fabric in which the overall percentage of bonded surface area of the nonwoven fabric is less than 12%, the surface area of the individual bond points is from about 0.10 to 0.60 square millimeters (mm²), the bond point packing value is greater than about 3.5 mmi, and the bond density is from about 20 to 60 individual point bonds per square centimeter (cm²) provides a nonwoven fabric having improved softness and abrasion resistance in comparison to a similar nonwoven fabric having a greater percentage of bonded surface area.

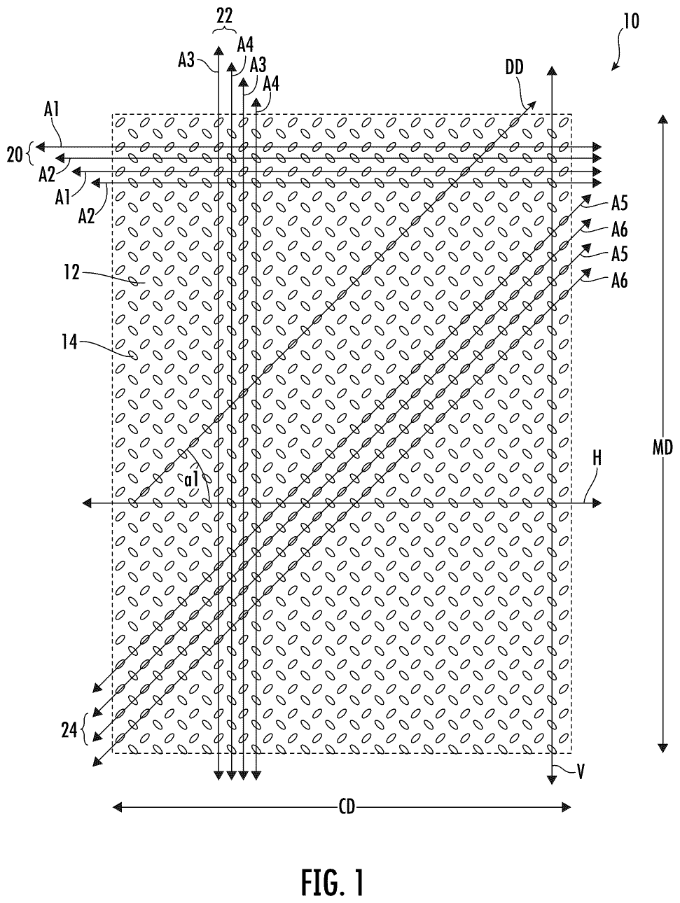

With reference to FIG. 1, a bonded nonwoven fabric in accordance with one or more embodiments of the invention is shown and broadly designated by reference character 10. The bonded nonwoven fabric 10 comprises a surface 12 comprising a plurality of individual point bonds 14 thereon. The bond points 14 are spaced apart from each other and are configured and arranged to define a first pattern comprising a plurality of arrays that extend in the machine direction (“MD”), cross direction (“CD”) and diagonal direction (“DD”) of the nonwoven fabric 10. The nonwoven fabric 10 also includes a vertical axis (“V”) that is substantially aligned with the machine direction of the nonwoven fabric 10, and a horizontal axis (“H) that is substantially aligned with the cross direction of the nonwoven fabric 10.

In the illustrated embodiment, the individual bond points 14 define regions of the nonwoven fabric 10 in which the fibers are thermally bonded together to form a coherent web.

The arrays extending in the cross direction of the nonwoven fabric 10 comprise a plurality of individual bond points 14 in which the bond points 14 are spaced apart from each other and extend laterally in the cross direction of the nonwoven fabric. In addition, the arrays extending in the cross direction comprise a plurality of array pairs 20 in which each array pair comprises a first array A1 defining a first member of the array pair 20 and a second array A2 defining a second member of the array pair. The plurality of array pairs 20 define a pattern in which first array A1 and second array A2 alternate in a repeating pattern in the machine direction of the nonwoven fabric.

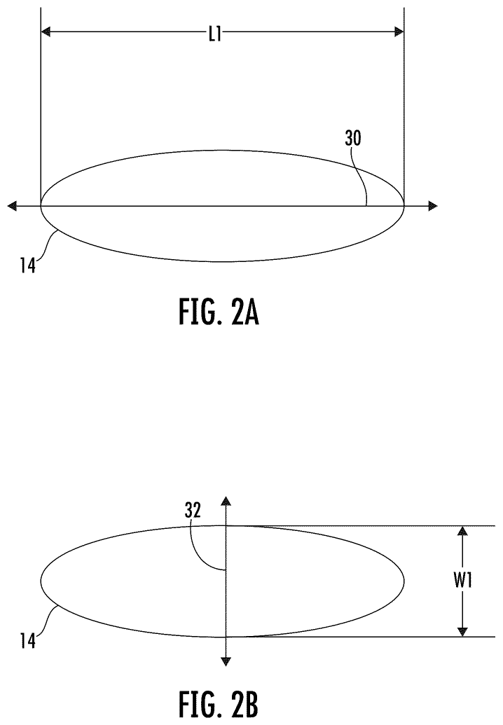

As shown in FIG. 1, the individual bond points 14 may have a generally oblong shape, such as oval-elliptical, rectangular, rod/bar, or the like. In an oblong shaped bond point, the bond point includes a major axis and a minor axis in which the major axis is of greater length than the minor axis (see, e.g., FIGS. 2A and 2B, reference characters 30, 32, respectively). In certain embodiments, the major axis of the bond points in the same array (e.g., first array A1) are oriented/aligned in the same direction while the major axis of the individual bond points in the array forming the second member (e.g., second array A2) of the array pair are oriented/aligned in an alignment that is oriented about 85° to 95° relative to the alignment of the major axis of bond points 14 in the first array A1. In certain embodiments, the individual bond points in first array are oriented/aligned in an alignment that is oriented about 86° to 94°, such as from about 87° to 93°, 88° to 92°, 86° to 94°, 89° to 91°, or 90° relative to the alignment of the major axis of bond points 14 in the second array A2.

Similarly, the arrays extending in the machine direction of the nonwoven fabric 10 comprise a plurality of individual bond points 14 in which the bond points 14 are spaced apart from each other and extend longitudinally in the machine direction of the nonwoven fabric. In addition, the arrays extending in the machine direction comprise a plurality of array pairs 22 in which each array pair comprises a third array A3 defining a first member of the array pair 22 and a fourth array A4 defining a second member of the array pair 22. The plurality of array pairs 22 define a pattern in which third array A3 and fourth array A4 alternate in a repeating pattern in the cross direction of the nonwoven fabric.

With respect to array pair 22, the major axis of the bond points 14 in the same array (e.g., third array A3) are oriented/aligned in the same direction while the major axis of the individual bond points in the array forming the second member (e.g., fourth array A4) of the array pair are oriented/aligned in an alignment that is oriented about 85° to 95° relative to the alignment of the major axis of bond points 14 in the third array A3. In certain embodiments, the individual bond points in third array A3 are oriented/aligned in an alignment that is oriented about 86° to 94°, such as from about 87° to 93°, 88° to 92°, 86° to 94°, 89° to 91°, or 90° relative to the alignment of the major axis of bond points 12 in the fourth array A4.

As noted previously, the nonwoven fabric may also include arrays of individual bond points 14 that extend in a diagonal direction of the nonwoven fabric. These diagonal arrays are also arranged in a plurality of array pairs 24 that extend across the surface of the nonwoven fabric at an angle that is diagonally aligned relative to the vertical and/or horizontal axis of the nonwoven fabric.

Each array pair 24 comprises a fifth array A5 and a sixth array A6 in which the plurality of array pairs 24 define a pattern in which fifth array A5 and sixth array A6 alternate in a repeating pattern in the diagonal direction of the nonwoven fabric.

As shown in FIG. 1, the major axis of each successive bond point 14 in the diagonally aligned arrays A5, A6 are rotated from about 85° to 95° relative to the alignment of the major axis of the preceding bond point 14 in the same array. In certain embodiments, the major axis of each successive bond point 14 in the diagonally oriented arrays A5, A6 are rotated from about 86° to 94°, such as from about 87° to 93°, 88° to 92°, 86° to 94°, 89° to 91°, or 90° relative to the alignment of the major axis of the preceding bond point 14 in the same array.

In certain embodiments, the intersection of the arrays extending in diagonal direction (DD) and the horizontal axis H of the nonwoven fabric 10 defines an angle a1. Angle a1 is typically between about 25° to 55°, and more typically, from about 28° to 48°, and even more typically, from about 30° to 45°.

In certain embodiments, angle a1 is greater than about 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, and 54°.

In certain embodiments, angle a1 is less than about 55°, 54°, 53°, 52°, 51°, 50°, 49°, 48°, 47°, 46°, 45°, 44°, 43°, 42°, 41°, 40°, 39°, 38°, 37°, 36°, 35°, 34°, 33°, 32°, 31°, 30°, 29°, 28°, 27°, and 26°.

In certain embodiments, the individual bond points having an oblong like shape have an average length that is from about 0.65 mm to 1.25 mm, and in particular, from about 0.70 to 1.20 mm, and more particularly, from about 1.15 to 0.72 mm, and even more particularly, from about 0.74 to 1.10 mm.

In a preferred embodiment, the individual bond points having an oblong like shape have an average length that is from about 0.74 to 0.78 mm, with an average length that is about 0.76 mm being somewhat more preferred.

In certain embodiments, the individual bond points having an oblong like shape have an average width that is from about 0.24 mm to 0.60 mm, and in particular, from about 0.25 to 0.55 mm, and more particularly, from about 0.27 to 0.50 mm, and even more particularly, from about 0.28 to 0.48 mm.

In preferred embodiments, the individual bond points having an oblong shape have an average width that is from about 0.24 to 0.36 mm, and in particular, from about 0.26 to 0.32 mm, and more particularly, from about 0.28 to 0.31 mm. In a slightly more preferred embodiment, the individual bond points have an average width that is about 0.30 mm.

In certain embodiments, the individual bond points have a length to width ratio that is from about 1.5 to 8, and in particular, from about 1.75 to 4, and more particularly, from about 2 to 2.8.

In certain embodiments, the average collective bond point distance ranges from about 1.10 to 2.25 mm. The average bond point distance refers to how closely the bond points are packed together in a given bonding pattern with respect to the machine, cross, and diagonal directions of the nonwoven fabric. The average bond point distance may be calculated from the average of distances between adjacent bonds in the machine direction, adjacent bonds in the cross direction, and adjacent bonds in the diagonal direction of the nonwoven fabric. In some embodiments, the average bond distance ranges from about 1.15 to 2.10 mm, and in particular, from about 1.20 to 2.0 mm. Unless otherwise specified, the distance between adjacent bonds is measured at the shortest distance between the two adjacent bonds.

It has further been discovered that a bonded nonwoven fabric comprising a bonding pattern having an average bond point packing value greater than about 3.5 mm⁻¹ provides improvements in softness and abrasion resistance as well as improvements in mechanical properties in comparison to a bonded nonwoven fabric having a greater % bonded area and an average bond point packing value that is less than 3.5 mm⁻¹.

The average bond point packing value for a given bonding pattern is calculated by dividing the collective average bond point distance by the average surface area of the bond point for the bonding pattern.

In certain embodiments, the bonded nonwoven fabric has an average bond point packing value from about 3.5 to 10 mm⁻¹, such as from about 4.5 to 9 mm⁻¹, and 5 to 7.5 mm⁻¹.

Referring to FIGS. 2A and 2B, the individual bond points 14 include a length L1 and width W1. The length of the bond point extends in a substantially straight line between opposite ends of the bond points, and defines a major axis 30 of the bond point. The width of the bond point is the measurement between opposite sides of the bond point, and defines a minor axis 32 of the bond point. The major axis 30 of an individual bond point extends through the farthest away points of the bond point, and the minor axis 32 extends through the closest points of the bond point. Generally, the major and minor axes of an individual bond point are substantially perpendicular to each other.

In the illustrated embodiments, the individual bond points have a generally oval-elliptical shape. It should be recognized that other shapes may be used, such as bond points having an oblong like shape and variations thereof. For example, the bond points may have a rectangular shape, bar/rod shape, oval-elliptical shape, and combinations thereof. In certain embodiments, the oblong shaped bond points may also be used in combination with other shaped bond points such as square, diamond or circular shaped bond points.

### Exemplary Embodiment A

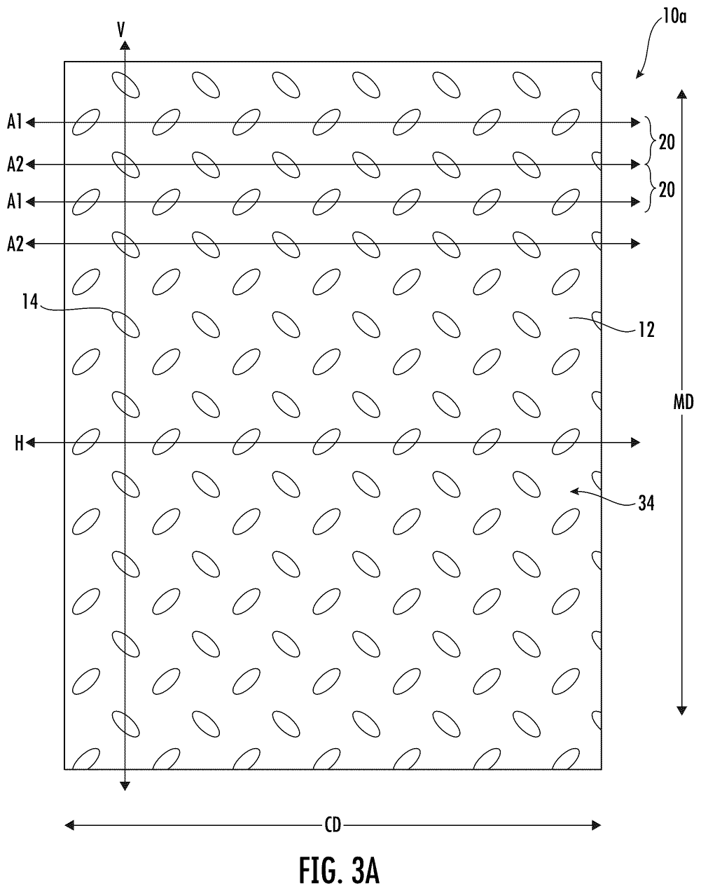

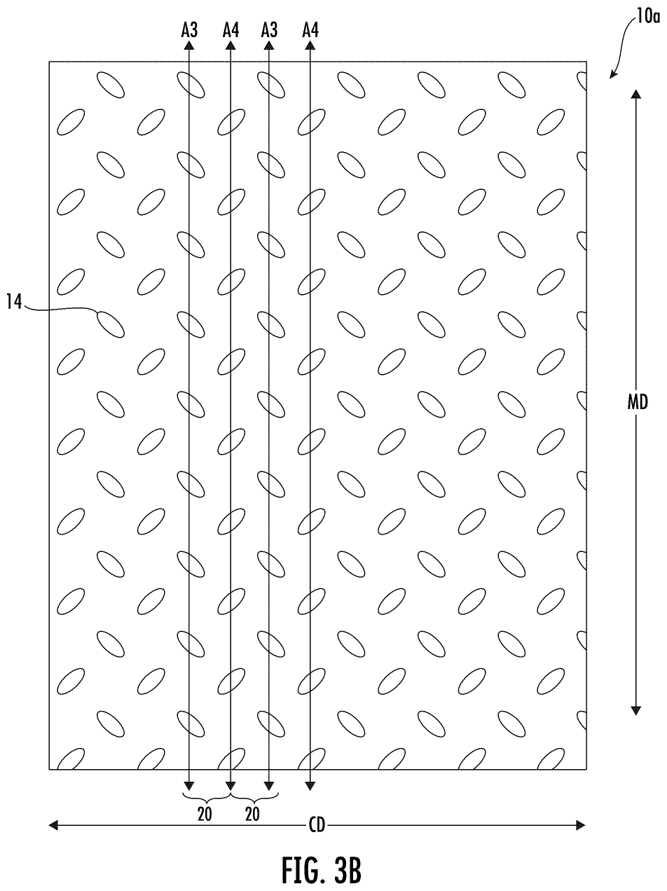

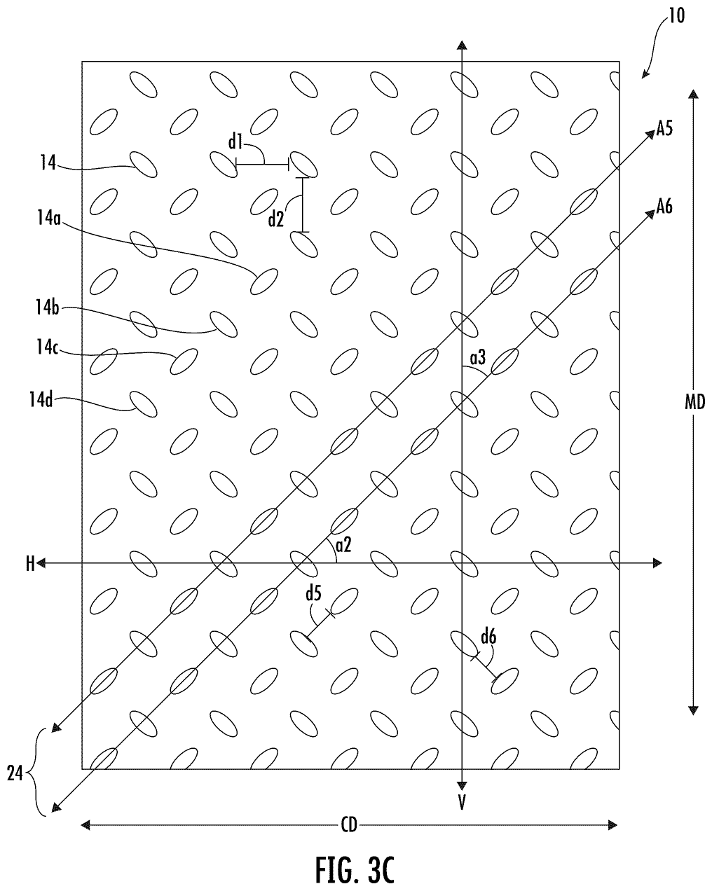

With reference to FIGS. 3A-3C, a preferred embodiment of the bonded nonwoven fabric is shown and generally indicated by reference character 10a. The nonwoven fabric 10a comprises a plurality of fibers that are point bonded to each other with a plurality of individual bond points 14 to form a coherent web. The nonwoven fabric includes a horizontal axis “H” that is substantially aligned with the cross direction “CD” of the nonwoven fabric and a vertical axis “V” that is substantially aligned with the machine direction “MD” of the nonwoven fabric.

In the embodiment of FIGS. 3A-3C, the overall percentage of bonded surface area of the nonwoven fabric is less than about 10%, the surface area of the individual bond points is from about 0.1 to 0.30 square millimeters (mm²), the bond point packing value is from about 6 to 8 mm⁻¹, and the bond density is from about 45 to 60 individual point bonds per square centimeter (cm²).

The plurality of individual bond points 14 define a first pattern 34 on the surface 12 of the nonwoven fabric 10a. As shown in FIG. 2A, the first pattern 34 comprises a series of alternating first and second arrays A1, A2 of individual bond points 14 that extend in the cross direction of the nonwoven fabric 10, and that are arranged in array pairs 20. In certain embodiments, the individual bond points 14 defining the second arrays of the first pattern are offset in the cross direction relative to adjacent bond points of the first arrays. In other words, adjacent bond points of the first and second arrays are not aligned with each other in the machine direction of the nonwoven fabric. This configuration and arrangement can be seen in FIG. 3A, where the vertical axis V only extends through bond points of the second array and does not extend through bond points of the first array. In the embodiment shown in FIG. 3A, the first and second arrays A1, A2 are substantially aligned with the cross direction of the nonwoven fabric 10a.

In a preferred embodiment, the individual bond points of the first arrays do not overlap in the machine direction with the individual bond points of the second arrays. However, it should be recognized that in some embodiments, the individual bond points of the first and second arrays may overlap in the machine direction with bond points in an adjacent array.

In certain embodiments, the individual bond points have an average length that is from about 0.65 mm to 0.85 mm, and in particular, from about 0.70 to 0.80 mm, and more particularly, from about 0.74 to 0.78 mm. In a preferred embodiment, the individual bond points have an average length that is about 0.76 mm.

In certain embodiments, the individual bond points have an average width that is from about 0.24 to 0.36 mm, and in particular, from about 0.26 to 0.32 mm, and more particularly, from about 0.28 to 0.31 mm. In a preferred embodiment, the individual bond points have an average width that is about 0.30 mm.

In certain embodiments, the individual bond points have a length to width ratio that is from about 2 to 3, and in particular, from about 2.15 to 2.85, and more particularly, from about 2.45 to 2.65.

Referring back to FIG. 3A, the lengths (e.g., major axes) of the individual bond points of the first array are typically aligned at an angle that is diagonal relative to the machine direction of the nonwoven fabric. That is, the intersection of the major axis 30 of individual bond points of the first array and the horizontal axis H of the nonwoven fabric defines an angle that is greater than 0° and less than 90°. In certain embodiments, the angle formed by the intersection of the horizontal axis H and the major axis of the individual bond point of the first array A1 is from about 43° to 47° degrees, and in particular, from about 44° to 46°, and more particularly, about 45°.

In certain embodiments, the individual bond points of the first array have their lengths (e.g., major axes) aligned in substantially the same direction as each other.

The lengths of the individual bond points of the second array are typically aligned at an angle that is diagonal relative to the machine direction of the nonwoven fabric. That is, the intersection of the major axis 30 of individual bond points of the second array and the horizontal axis H of the nonwoven fabric defines an angle that is greater than 0° and less than 90°. In certain embodiments, the angle formed by the intersection of the horizontal axis H and the major axis of the individual bond point of the second array A2 is from about 43° to 47° degrees, and in particular, from about 44° to 46°, and more particularly, about 45°.

In certain embodiments, the lengths (e.g., major axis) of the individual bond points of the second array are typically rotated from about 88° to 92°, such as from about 89° to 91°, and more particularly, about 90° relative to the alignment of the lengths (e.g., major axis) of the individual bond points of the first array.

In a preferred embodiment, the intersection of a line segment extending along the major axis of individual bond points of the first array and a line segment extending along the major axis of individual bond points of the second array defines an angle that is about 90°.

In certain embodiments, the individual bond points of the second array have their lengths (e.g., major axes) aligned in substantially the same direction as each other.

With reference to FIG. 3B, the first pattern 30 of bond points further comprises a series of alternating third and fourth arrays A3, A4 of individual bond points 14 that extend in the machine direction of the nonwoven fabric 10, and that arranged in array pairs 22. In the embodiment shown in FIG. 3B, the third and fourth arrays A3, A4 are substantially aligned with the machine direction of the nonwoven fabric 10a. Similar, to the first and second arrays A1, A2, the individual bond points 14 defining the fourth arrays of the first pattern are offset in the machine direction relative to adjacent bond points of the third arrays. In other words, adjacent bond points of the third and fourth arrays are not aligned with each other in the machine direction of the nonwoven fabric.

In certain embodiments, the lengths of the individual bond points of the third array A3 are typically aligned at an angle that is diagonal relative to the vertical axis of the nonwoven fabric. That is, the intersection of the major axis 30 of individual bond points of the third array and the vertical axis V of the nonwoven fabric defines an angle that is greater than 0° and less than 90°. In certain embodiments, the angle formed by the intersection of the horizontal axis V and the major axis of the individual bond point of the third array A3 is from about 43° to 47° degrees, and in particular, from about 44° to 46°, and more particularly, about 45°.

In certain embodiments, the individual bond points of the third array A3 have their lengths (e.g., major axes) aligned in substantially the same direction as each other.

Similarly, the lengths of the individual bond points of the fourth array are typically aligned at an angle that is diagonal relative to the vertical axis of the nonwoven fabric. That is, the intersection of the major axis 30 of individual bond points of the fourth array and the vertical axis V of the nonwoven fabric defines an angle that is greater than 0° and less than 90°. In certain embodiments, the angle formed by the intersection of the vertical axis V and the major axis of the individual bond point of the fourth array A4 is from about 43° to 47° degrees, and in particular, from about 44° to 46°, and more particularly, about 45°.

In certain embodiments, the lengths (i.e., major axis) of the individual bond points of the fourth array are typically rotated from about 88 to 92°, such as from about 89° to 91°, and more particularly, about 90° relative to the alignment of the lengths (i.e., major axis) of the individual bond points of the third array.

In a preferred embodiment, the intersection of a line segment of the major axis of individual bond points of the third array and a line segment of the major axis of individual bond points of the fourth array define an angle that is about 90°.

In certain embodiments, the individual bond points of the fourth array have their lengths (e.g., major axes) aligned in substantially the same direction as each other.

Turning now to FIG. 3C, the first pattern 34 of bond points further comprises a series of alternating fifth and sixth arrays A5, A6 in which the arrays extend diagonally across the surface of the nonwoven fabric relative to the machine direction of the fabric. The fifth and sixth arrays A5, A6 arrays are arranged in array pairs 24. As shown in FIG. 3C, the intersection of the fifth or sixth arrays with the horizontal axis H defines an angle a2. Generally, angle a2 is from about 40° to 50°, and in particular, from about 42° to 48°, more particularly from about 43° to 47°, and even more particularly, from about 44° to 46°. In a preferred embodiment, angle a2 is about 45°.

Similarly, the intersection of the fifth and sixth arrays with the vertical axis V defines an angle a3. Generally, angle a3 is from about 40° to 50°, and in particular, from about 42° to 48°, more particularly from about 43° to 47°, and even more particularly, from about 44° to 46°. In a preferred embodiment, angle a3 is about 45°.

As shown in FIG. 3C, each successive bond point in the fifth and sixth arrays are rotated approximately 90° relative to the preceding bond point in the same array. In this regard, it can be seen that the lengths of bond points 14a, 14c are aligned substantially in the same direction relative to the horizontal axis H of the nonwoven fabric 10a, and that the lengths of bond points 14b, 14d are rotated approximately 90° relative to the lengths of bond points 14a, 14c. In this way, the major axis of each successive bond point in the same array is substantially perpendicular to the major axis of the preceding or succeeding bond point in the same array.

Continuing to refer to FIG. 3C, the distance d1 between adjacent bond points in the same array in cross direction of the first and second arrays may range from about 1.40 to 1.60 mm, and in particular, from about 1.45 to 1.55, and more particularly, from about 1.48 to 1.52. In a preferred embodiment, the distance d1 between adjacent bond points in the cross direction of the first and second arrays is about 1.51 mm.

In certain embodiments, the distance d2 between adjacent bond points in the machine direction in the same array of the third and fourth arrays may range from about 1.40 to 1.60 mm, and in particular, from about 1.45 to 1.55, and more particularly, from about 1.48 to 1.52 mm. In a preferred embodiment, the distance d2 between adjacent bond points in the same array in the machine direction of the third and fourth arrays is about 1.51 mm.

In certain embodiments, the distance d5 between adjacent bond points in the same arrays of the fifth or sixth arrays may range from about 0.70 to 0.95 mm, and in particular, from about 0.75 to 0.90, and more particularly, from about 0.80 to 0.85. In a preferred embodiment, the distance d5 between adjacent bond points in the same array of the fifth or sixth arrays is about 0.82 to 0.84 mm.

Similarly, for diagonally aligned arrays that are rotated approximately 90° relative to the fifth and sixth arrays (not identified by reference characters), the distance d6 between adjacent bond points in the same array may range from about 0.70 to 0.95 mm, and in particular, from about 0.75 to 0.90, and more particularly, from about 0.80 to 0.85 mm. In a preferred embodiment, the distance d6 between adjacent bond points in the same array is about 0.82 to 0.84 mm.

In certain embodiments, the average collective bond point distance for the embodiments of FIGS. 3A-3C may ranges from about 1.15 to 1.45 mm. As discussed previously, the average bond point distance is calculated from the average of distances between bond points in the machine, cross, and diagonal direction of the nonwoven fabric. For example, the average collective bond point distance may be calculated from the average distance of d1, d2, d5, and d6.

In certain embodiments, the average collective bond point distance is from about 1.20 mm to 1.40 mm, and in particular, from about 1.25 to 1.35 mm, and more particularly, from about 1.26 to 1.30 mm, with an average distance of 1.27 to 1.29 mm being somewhat more preferred.

In certain embodiments in accordance with the embodiments of FIGS. 3A-3C, the bonded nonwoven fabric has an average bond point packing value from about 6 to 10.0 mm⁻¹, such as from about 6.5 to 9.5 mm⁻¹, and 7.0 to 8.0 mm⁻¹. In a preferred embodiment, the bonded nonwoven fabric has an average bond point packing value from about 7.0 to 7.25 mm⁻¹.

More particularly, in certain embodiments in accordance with the embodiments of FIGS. 3A-3C, the bonded nonwoven fabric has an average bond point packing value greater than 6.0 mm⁻¹, greater than 6.1 mm⁻¹, greater than 6.2 mm⁻¹, greater than 6.3 mm⁻¹, greater than 6.4 mm⁻¹, greater than 6.5 mm⁻¹, greater than 6.6 mm⁻¹, greater than 6.7 mm⁻¹, greater than 6.8 mm⁻¹, greater than 6.9 mm⁻¹, greater than 7.0 mm⁻¹, greater than 7.1 mm⁻¹, greater than 7.2 mm⁻¹, greater than 7.3 mm⁻¹, greater than 7.4 mm⁻¹, greater than 7.5 mm⁻¹, greater than 7.6 mm⁻¹, greater than 7.7 mm⁻¹, greater than 7.8 mm⁻¹, greater than 7.9 mm⁻¹, greater than 8.0 mm⁻¹, greater than 8.1 mm⁻¹, greater than 8.2 mm⁻¹, greater than 8.3 mm⁻¹, greater than 8.4 mm⁻¹, greater than 8.5 mm⁻¹, greater than 8.6 mm⁻¹, greater than 8.7 mm⁻¹, greater than 8.8 mm⁻¹, greater than 8.9 mm⁻¹, greater than 9.0 mm⁻¹, greater than 9.1 mm⁻¹, greater than 9.2 mm⁻¹, greater than 9.3 mm⁻¹, greater than 9.4 mm⁻¹, greater than 9.5 mm⁻¹, greater than 9.6 mm⁻¹, greater than 9.7 mm⁻¹, greater than 9.8 mm⁻¹, greater than 9.9 mm⁻¹, and greater than 10.0 mm⁻¹.

In certain embodiments, in accordance with the embodiments of FIGS. 3A-3C, the bonded nonwoven fabric has an average bond point packing value of less than 10.0 mm⁻¹, less than 9.9 mm⁻¹, less than 9.8 mm⁻¹, less than 9.7 mm⁻¹, less than 9.6 mm⁻¹, less than 9.5 mm⁻¹, less than 9.4 mm⁻¹, less than 9.3 mm⁻¹, less than 9.2 mm⁻¹, less than 9.1 mm⁻¹, less than 9.0 mm⁻¹, less than 8.9 mm⁻¹, less than 8.8 mm⁻¹, less than 8.7 mm⁻¹, less than 8.6 mm⁻¹, less than 8.5 mm⁻¹, less than 8.4 mm⁻¹, less than 8.3 mm⁻¹, less than 8.2 mm⁻¹, less than 8.1 mm⁻¹, less than 7.0 mm⁻¹, less than 6.9 mm⁻¹, less than 6.8 mm⁻¹, less than 6.7 mm⁻¹, less than 6.6 mm⁻¹, less than 6.5 mm⁻¹, less than 6.4 mm⁻¹, less than 6.3 mm⁻¹, less than 6.2 mm⁻¹, less than 6.1 mm⁻¹, less than 6.0 mm⁻¹, less than 5.9 mm⁻¹, less than 5.8 mm⁻¹, less than 5.7 mm⁻¹, less than 5.6 mm⁻¹, less than 5.5 mm⁻¹, less than 5.4 mm⁻¹, less than 5.3 mm⁻¹, less than 5.2 mm⁻¹, less than 5.1 mm⁻¹, and less than 5.0 mm⁻¹

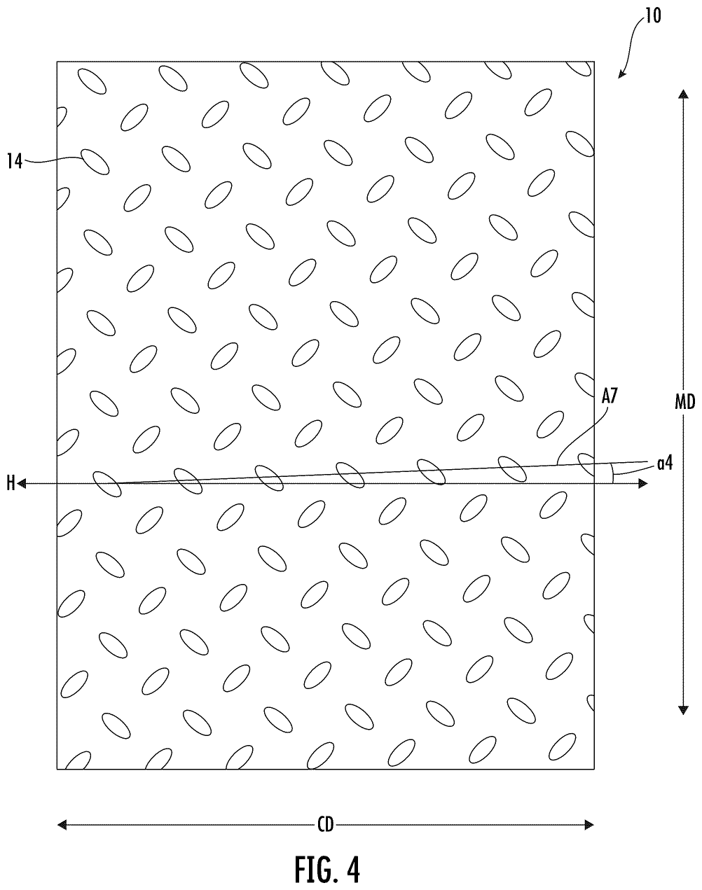

In certain embodiments, the arrays extending in the cross direction of the nonwoven fabric may not be parallel with the horizontal axis H of the nonwoven fabric. In this regard, FIG. 4 illustrates an embodiment in which the first bond pattern comprises a plurality of arrays of individual bond points in which the arrays are not aligned with the horizontal axis of the nonwoven fabric 10. As shown, array A7 defines an array in which the individual bond points 16 extend in the cross direction of the nonwoven fabric at an angle that is greater than 0° relative to the horizontal axis. In particular, the intersection of array A7 and the horizontal axis defines angle a3 that may range from greater than 0° to less than 6°. In a preferred embodiment, angle a3 is from about 0.5° to 4°, and in particular, from about 1° to 3°, with an angle of about 2° being preferred.

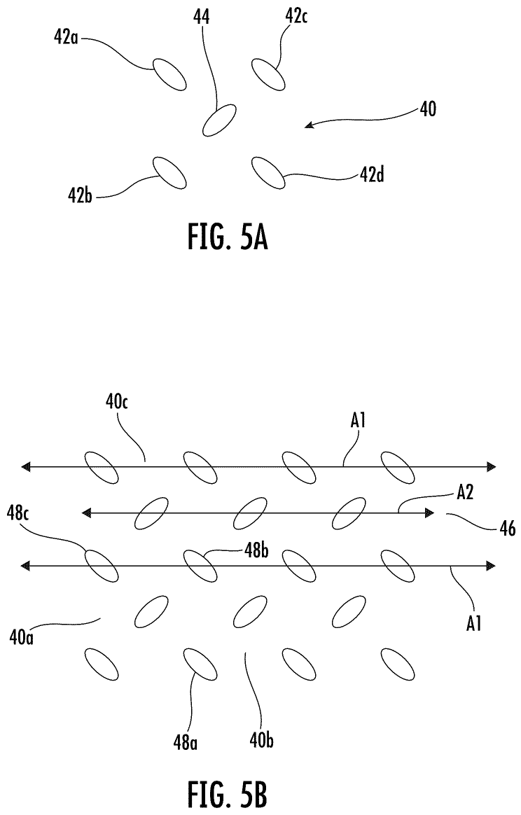

In certain embodiments, the first pattern defines a plurality of second patterns within the first pattern. In this regard, FIGS. 5A and 5B illustrate a second pattern 40 comprising individual bond points that collectively define a bond pattern having a quincunx like shape. As seen in FIGS. 5A and 5B, three adjacent arrays (e.g., A1, A2, A1) of individual bond points further define a plurality of bond patterns in both the machine and cross directions having a quincunx like shape in which four individual bond points defining corners (40a, 40b, 40c, 40d) of the quincunx like bond pattern 40 share a directional orientation that is substantially the same relative to the cross or machine directions of the nonwoven fabric, and wherein an individual bond point 44 defining a center point of the quincunx like bond pattern has a directional orientation that is rotated from about 88° to 92° (e.g., from about 89° to 91°, and in particular, about 90°) relative to the directional orientation of the individual bond points defining the corners of the quincunx like bond pattern.

FIG. 5B illustrates a partial bond pattern 46 of the first bond pattern having a plurality of quincunx like bond pattern extending in both the machine and cross directions of the nonwoven fabric. Each quincunx like bond pattern shares corner bond points with adjacent quincunx like bond patterns in the machine and cross directions. In this regard, quincunx like bond pattern 40a shares corner bond points 48a and 48b with quincunx like bond pattern 40b in the cross direction. Similarly, quincunx like bond pattern 40a shares corner bond points 48c, 48b with adjacent quincunx like bond pattern 40c in the machine direction of the nonwoven fabric.

As discussed previously, the inventors of the present disclosure have surprisingly discovered that abrasion resistant is improved in comparison to similarly prepared nonwoven fabrics comprising oval bonds with a higher percentage of bonded area of the nonwoven fabric. In particular, embodiments of the present invention provide improvements in abrasion resistance while having a lower percentage of bonded area in comparison to similar nonwoven fabrics.

The surface area of each individual bond point 14 in accordance with embodiments shown in FIGS. 3A-3C, is typically from about 0.10 to 0.40 mm², and more typically, from about 0.12 to 0.3 mm², and even more typically, about 0.15 to 0.25 mm². In a preferred embodiment, the surface area of each individual bond point is about 0.18 mm².

In certain embodiments, the average surface area of each individual bond point 14 in accordance with embodiments shown in FIGS. 3A-3C, is greater than 0.1, greater than 0.11, greater than, 0.12, greater than 0.13, greater than 0.14, greater than 0.15, greater than 0.16, greater than 0.17, greater than 0.17, greater than 0.18, greater than 0.19, greater than 0.20, greater than 0.21, greater than 0.22, greater than 0.23, greater than 0.24, greater than 0.25, greater than 0.26, greater than 0.27, greater than 0.28, greater than 0.29, and greater than 0.30.

In certain embodiments, the average surface area of each individual bond point 14 in accordance with embodiments shown in FIGS. 3A-3C, is less than 0.30, less than 0.29, less than, 0.28, less than 0.27, less than, 0.26, less than 0.25, less than, 0.25, less than 0.23, less than, 0.22, less than 0.21, less than, 0.20, less than 0.19, less than, 0.18, less than 0.17, less than, 0.16, less than 0.15, less than, 0.14, less than 0.13, less than, 0.12, less than 0.11, and less than, 0.10.

In certain embodiments of the invention, the number of individual bond points per cm² is from about 45 to 60, and in particular, from about 50 to 58, and more particularly, from about 54 to 56.

In some embodiments, the percent bonded area of the nonwoven fabric is from about 9 to 10.5%, and in particular, from about 9.5 to 10.4%, and more particularly, from about 9.8 to 10.2%. In a preferred embodiment, the percent bonded area of the nonwoven fabric from about 9.9 to 10%.

### Exemplary Embodiment B

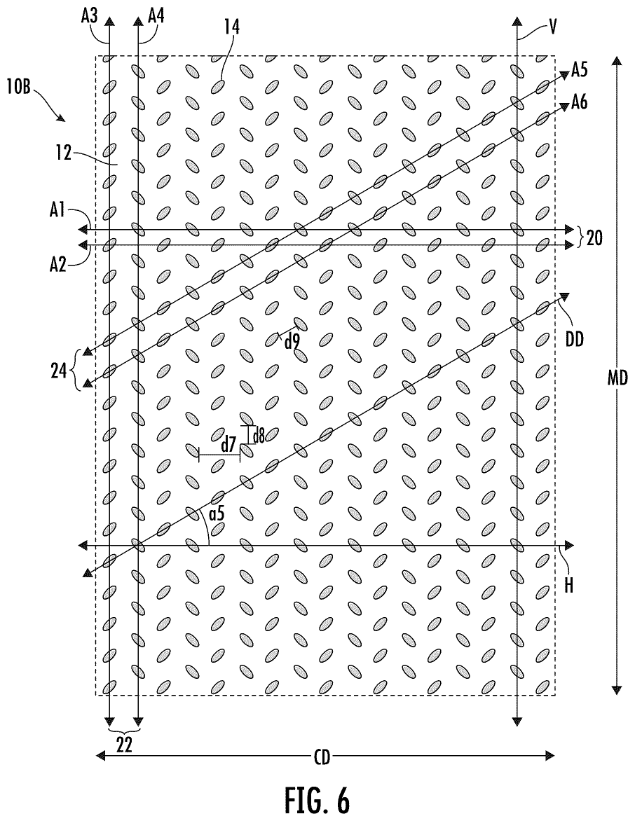

With reference to FIG. 6, an embodiment of the nonwoven fabric is shown and broadly designated by reference character 10b. As in the embodiments shown in FIGS. 3A-3C, the nonwoven fabric 10b comprises a plurality of fibers that are point bonded to each other with a plurality of individual bond points 14 to form a coherent web. The nonwoven fabric further includes a surface 12 comprising a plurality of individual bond points 14 thereon. The bond points 14 are spaced apart from each other and are configured and arranged to define a plurality of arrays that extend in the machine direction (“MD”), cross direction (“CD”) and diagonal direction (“DD”) of the nonwoven fabric 10b. The nonwoven fabric 10b also includes a vertical axis (“V”) that is substantially aligned the machine direction of the nonwoven fabric 10, and a horizontal axis (“H) that is substantially aligned with the cross direction of the nonwoven fabric 10.

Similar to the previously discussed embodiment, the nonwoven fabric 10b further comprises pairs (20) of alternating first and second arrays (reference characters A1, A2) extending laterally in the cross direction of the nonwoven fabric; pairs (22) of alternating third and fourth arrays (reference characters A3, A4) extending longitudinally in the machine direction of the nonwoven fabric; and pairs (24) of alternating fifth and sixth arrays (reference characters A5, A6) extending diagonally across the surface of the nonwoven fabric relative to the machine direction of the nonwoven fabric.

In the embodiment illustrated in FIG. 6, the lengths (e.g., major axes) of the individual bond points of the first array are typically aligned at an angle that is diagonal relative to the machine direction of the nonwoven fabric. That is, the intersection of the major axis 30 of individual bond points of the first array and the horizontal axis H of the nonwoven fabric defines an angle that is greater than 0° and less than 90°. In certain embodiments, the angle formed by the intersection of the horizontal axis H and the major axis of the individual bond point of the first array is from about 43° to 47° degrees, and in particular, from about 44° to 46°, and more particularly, about 45°.

In certain embodiments, the individual bond points of the first array have their lengths (e.g., major axes) aligned in substantially the same direction as each other.

The lengths (e.g., major axes) of the individual bond points of the second array are typically aligned at an angle that is diagonal relative to the machine direction of the nonwoven fabric. That is, the intersection of the major axis 30 of individual bond points of the second array and the horizontal axis H of the nonwoven fabric defines an angle that is greater than 0° and less than 90°. In certain embodiments, the angle formed by the intersection of the horizontal axis H and the major axis of the individual bond point of the second array is from about 43° to 47° degrees, and in particular, from about 44° to 46°, and more particularly, about 45°.

In certain embodiments, the lengths (e.g., major axis) of the individual bond points of the second array are typically rotated from about 88° to 92°, such as from about 89° to 91°, and more particularly, about 90° relative to the alignment of the lengths (e.g., major axis) of the individual bond points of the first array.

The intersection of the array extending in the diagonal direction (DD) (e.g., arrays A5, A6) with the horizontal axis H defines an angle a5. Generally, angle a5 is from about 28° to 36°, and in particular, from about 29° to 35°, more particularly from about 30° to 34°, and even more particularly, from about 31° to 33°. In a preferred embodiment, angle a5 is about 32°.

In the embodiment of FIG. 6, the percentage of bonded surface area of the nonwoven fabric is less than about 12%, the average surface area of the individual bond points is from about 0.2 to 0.6 square millimeters (mm²), and the bond density is from about 30 to 30 individual point bonds per square centimeter (cm²).

In certain embodiments, the percentage of bonded surface area of the nonwoven fabric is from about 10 to 10.5%, the average surface area of the individual bond points is from about 0.35 to 0.45 mm², the bond point packing value is from about 4.5 to 6.5 mm⁻¹, and the bond density is from about 24 to 26 individual point bonds per square centimeter (cm²).

In certain embodiments, the individual bond points have an average length that is from about 1.04 mm to 1.14 mm, and in particular, from about 1.06 to 1.12 mm, and more particularly, from about 1.08 to 1.10 mm. In a preferred embodiment, the individual bond points have an average length that is about 1.09 mm.

In certain embodiments, the individual bond points have an average width that is from about 0.44 to 0.50 mm, and in particular, from about 0.45 to 0.49 mm, and more particularly, from about 0.46 to 0.48 mm. In a preferred embodiment, the individual bond points have an average width that is about 0.47 mm.

In certain embodiments, the average surface area of the individual bond points is from about 0.3 to 0.8 square millimeters (mm²), and in particular, about 0.35 to 0.7 mm², and more particularly, from about 0.38 to 0.5 mm². In some embodiments, the average surface area of the individual bond points is about 0.4 mm².

In some embodiments, the bond density (number of individual bond points per square cm) is from about 20 to 30, and in particular, from about 22 to 28, and more particularly, 24 to 26 bond points per cm².

The distance d7 between adjacent point bonds in arrays extending in the cross direction (e.g., arrays A1, A2) in the same array is typically from about 2.90 to 3.2 mm, and in particular, from about 2.95 to 3.15 mm, and more particularly, from about 3.0 to 3.12 mm, and even more particularly, from about 3.05 to 3.10 mm. In a preferred embodiment, the distance d7 between adjacent point bonds in arrays extending in the cross direction is about 3.08 mm.

The distance d8 between adjacent point bonds in arrays extending in the machine direction (e.g., arrays A3, A4) in the same array is typically from about 1.65 to 1.85 mm, and in particular, from about 1.70 to 1.80 mm, and more typically, from about 1.72 to 1.78 mm. In a preferred embodiment, the distance d8 between adjacent point bonds in arrays extending in the machine direction is about 1.75 mm.

The distance d9 between adjacent point bonds in arrays extending in the diagonal direction (e.g., arrays A5, A6) in the same array is typically from about 1.25 to 1.55 mm, and in particular, from about 1.30 to 1.45 mm, and more typically, from about 1.34 to 1.44 mm. In a preferred embodiment, the distance d9 between adjacent point bonds in arrays extending in the machine direction in the same array is about 1.38 to 1.40 mm.

In certain embodiments, the average collective bond point distance for the embodiments in accordance with FIG. 6 may ranges from about 1.90 to 2.25 mm. As discussed previously, the average bond point distance is calculated from the average of distances between bond points in the machine, cross, and diagonal direction of the nonwoven fabric. For example, the average collective bond point distance may be calculated from the average distance of d7, d8, and d9.

In certain embodiments, the average collective bond point distance is from about 1.95 mm to 2.20 mm, and in particular, from about 2.05 to 2.15 mm, and more particularly, from about 2.06 to 2.12 mm, with an average distance of 2.07 to 2.08 mm being somewhat more preferred.

In certain embodiments in accordance with the embodiments of FIG. 6, the bonded nonwoven fabric has an average bond point packing value from about 4 to 6.5 mm⁻¹, such as from about 4.5 to 6 mm⁻¹, and 4.75 to 5.5 mm⁻¹. In a preferred embodiment, the bonded nonwoven fabric has an average bond point packing value from about 5.0 to 5.25 mm⁻¹.

### Exemplary Embodiment C

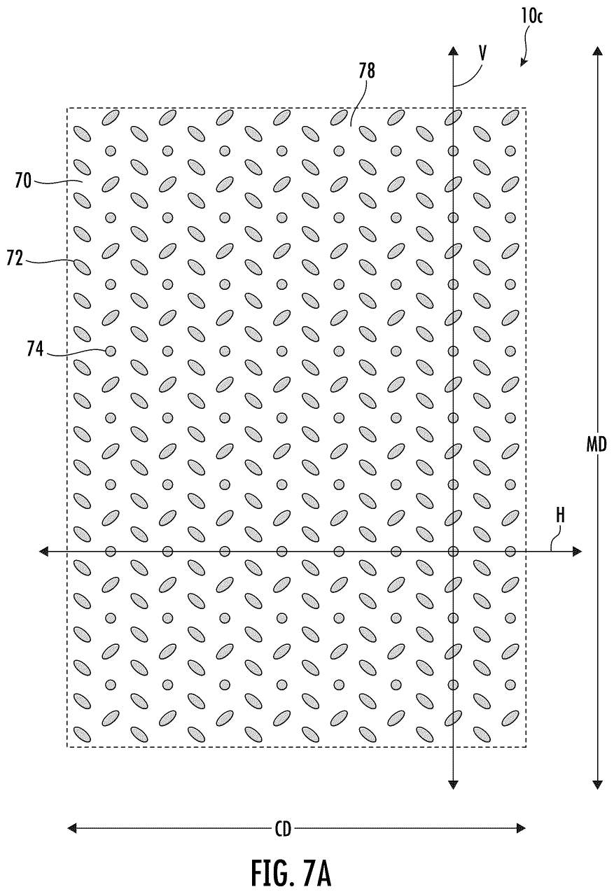

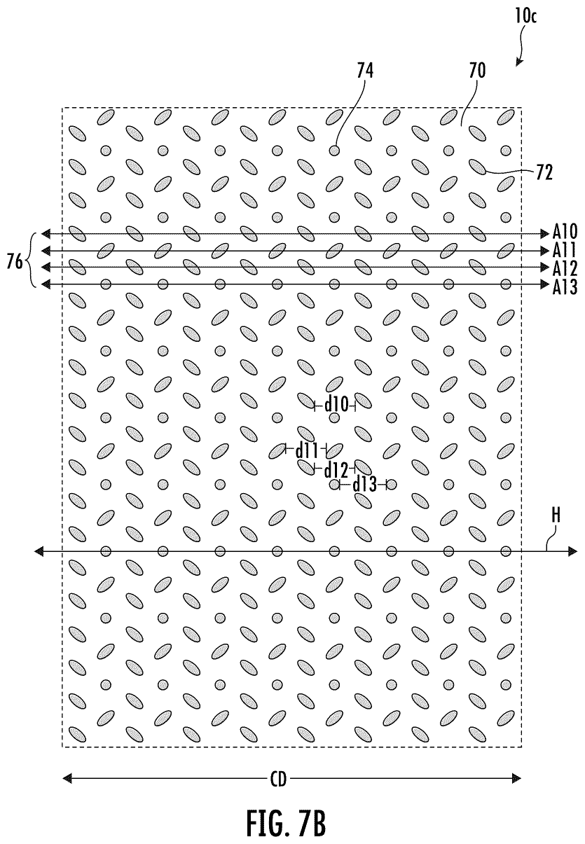

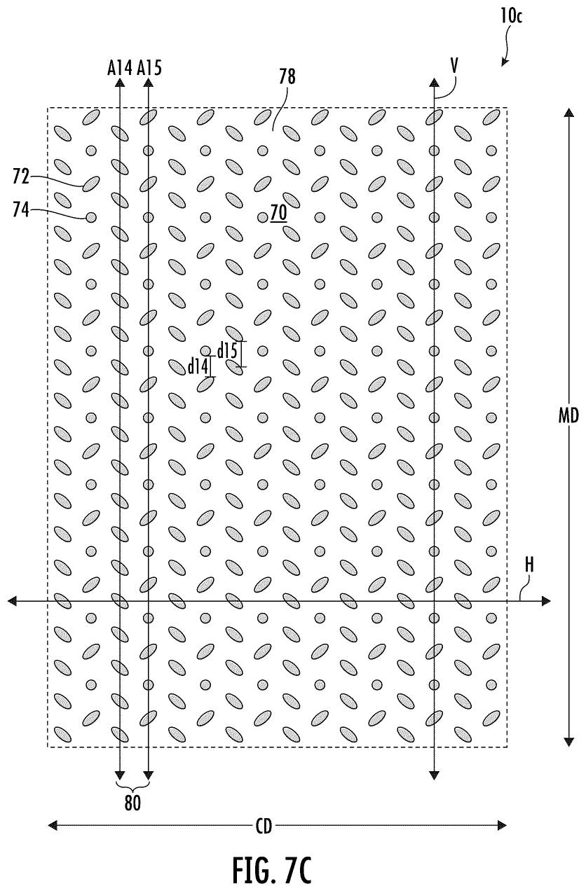

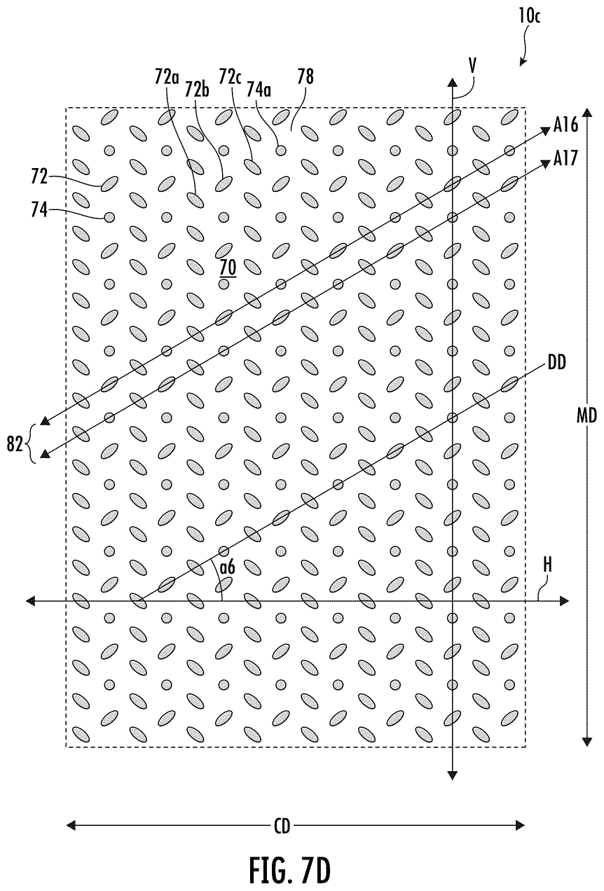

Turning now to FIGS. 7A-7D, an additional embodiment of a bonded nonwoven fabric in accordance with at least one embodiment of the disclosure is illustrated and designated by reference character 10c.

As in the embodiments shown in FIG. 1, the nonwoven fabric 10c comprises a plurality of fibers that are point bonded to each other with a plurality of individual bond points 72, 74 to form a coherent web. The nonwoven fabric further includes a surface 70 comprising a plurality of individual bond points 72, 74 thereon that collectively define a bonded pattern 78 on the surface of the nonwoven fabric 10c. The bond points 14 are spaced apart from each other and are configured and arranged to define a plurality of arrays that extend in the machine direction (“MD”), cross direction (“CD”) and diagonal direction (“DD”) of the nonwoven fabric 10c. The nonwoven fabric 10c also includes a vertical axis (“V”) that is substantially aligned the machine direction of the nonwoven fabric 10c, and a horizontal axis (“H) that is substantially aligned with the cross direction of the nonwoven fabric 10c.

The embodiment of FIG. 7B comprises a plurality of a first array set 76 in which each of the arrays of the first array set 76 comprises a plurality of individual bond points that extend laterally in the cross direction of the nonwoven fabric 10c. The first array set 76 includes four arrays (A10, A11, A12, A13) that each extend laterally in the cross direction of the nonwoven fabric 10c and are successively spaced apart from each other in the machine direction of the nonwoven fabric 10c. That is, when array A10 is the first array of first array set 76, then the next successive array in the machine direction of the nonwoven fabric 10c will be array A11, and the next successive array following array A11 will be array A12, and finally, the next successive array of first array set 76 following array A12 is array A13. At this point, the pattern repeats itself in the machine direction of the nonwoven fabric 10c.

In the illustrated embodiment, the array A10 comprises a plurality of spaced apart individual bond points 72 having a generally oblong shape, such as an oval-elliptical shape. Additional variations of oblong shaped bond points that may be used in the embodiments of the invention are discussed previously. The lengths (e.g., major axes) of the individual bond points of the first array A10 are typically aligned at an angle that is diagonal relative to the machine direction of the nonwoven fabric. That is, the intersection of the major axis 30 of individual bond points 72 of the first array and the horizontal axis H of the nonwoven fabric defines an angle that is greater than 0° and less than 90°. In certain embodiments, the angle formed by the intersection of the horizontal axis H and the major axis of the individual bond point of the first array A10 is from about 26° to 34° degrees, and in particular, from about 28° to 32°, and more particularly, 29° to 31°. In a somewhat more preferred embodiment, the angle formed by the intersection of the horizontal axis H and the major axis of the individual bond point of the first array A10 is about 30°.

In certain embodiments, the individual bond points 72 of the array A10 have their lengths (e.g., major axes) aligned in substantially the same direction as each other.

Following, array A10 is array A11. Array A11 comprises a plurality of spaced apart individual bond points 72 having a generally oblong shape, such as an oval-elliptical shape. Similar to array A10, the lengths (e.g., major axes) of the individual bond points of array A11 are typically aligned at an angle that is diagonal relative to the machine direction of the nonwoven fabric. In certain embodiments, the angle formed by the intersection of the horizontal axis H and the major axis of the individual bond points 74 of the array A11 is from about 26° to 34° degrees, and in particular, from about 28° to 32°, and more particularly, 29° to 31°. In a somewhat more preferred embodiment, the angle formed by the intersection of the horizontal axis H and the major axis of the individual bond point of the first array A11 is about 30°.

However, in array A11 the alignment of the lengths (e.g., major axes) of the individual bond points of array A11 are rotated in a directional orientation that is opposite to the alignment of array A10 relative to the vertical axis of the nonwoven fabric. In particular, the lengths (e.g., major axis) of the individual bond points 72 of the array A11 are typically rotated from about 85° to 95°, such as from about 88° to 92°, and more particularly, about 88° to 92° relative to the alignment of the lengths (e.g., major axis) of the individual bond points of array A10.

Array A12 successively follows array A11 in the machine direction of the nonwoven fabric 10c. The configuration and arrangement of the bond points of array A12 are substantially similar to that of array A10. That is, the lengths (e.g., major axes) of bond points 72 of arrays A10, A12 are substantially aligned in the same direction relative to the horizontal axis of the nonwoven fabric 10c. Further, the adjacent bond points in arrays A10, A12 are substantially aligned in the machine direction of the nonwoven fabric 10c.

It can also be seen that the position of the individual bond points in array A11 are offset in the cross direction from adjacent bond points in arrays A10 and A12. In other words, the bond points of array A11 are not aligned in the machine direction with the bond points of arrays A10 and A12.

The average distances d10 between adjacent bond points 74 within the same array of array A10 may range from 2.2 to 2.6 mm, and in particular, from 2.3 to 2.55 mm, and more particularly, from about 2.45 to 2.50 mm. Similarly, average distances d12 between adjacent bond points 74 within the same array of array A12 may range from 2.3 to 2.6 mm, and in particular, from 2.3 to 2.55 mm, and more particularly, from about 2.45 to 2.50 mm. The average distances d11 between adjacent bond points 74 within the same array of array A11 may range from 2.2 to 2.6 mm, and in particular, from 2.3 to 2.55 mm, and more particularly, from about 2.45 to 2.50 mm.

Array A13 comprises a plurality of circular/square shaped bond points 74 that extend laterally in the cross direction of the nonwoven fabric 10c. Typically bond points 74 have a length to width ratio that is from about 0.9:1.1 to 1:1.

In certain embodiments, the individual bond points 74 of array A13 are substantially aligned in the machine direction with adjacent bond points 74 in array A11.

The average distances d13 between adjacent bond points 74 within the same array of array A13 may range from 2.45 to 2.75 mm, and in particular, from 2.50 to 2.70 mm, and more particularly, from about 2.55 to 2.65 mm, with a distance from about 2.58 to 2.62 mm being somewhat more preferred.

The average distances d14 between adjacent bond points in the diagonal direction of the nonwoven fabric may range from about 1.20 to 1.50 mm, and in particular, from about 1.25 to 1.45 mm, and more particularly, from about 1.30 to 1.40 mm. In a preferred embodiment, the average distance d14 is from about 1.32 to 1.36 mm.

With reference to FIG. 7C, bonded pattern 78 may further comprise a second array set 80 comprising a pair of alternating arrays extending longitudinally in the machine direction of the nonwoven fabric 10c. The second array set 80 comprises a pair of alternating arrays A14, array A15 that are spaced from each other in the cross direction and extend in the machine direction of the nonwoven fabric. Each array comprises a plurality of spaced apart individual bond points 72, 74.

Array A14 comprise a plurality of generally oblong shaped bond points 72, such as an elliptical oval shaped bond point. Other variations of oblong shaped bond points are discussed previously. In certain embodiments and as shown in FIG. 7C, the oblong shaped bond points 72 of array A14 are aligned in substantially the same direction with respect to the machine direction of the nonwoven fabric. In some embodiments, the angle formed by the intersection of the horizontal axis H and the major axis of the individual bond point of array A14 may be from about 26° to 34° degrees, and in particular, from about 28° to 32°, and more particularly, 29° to 31°. In a somewhat more preferred embodiment, the angle formed by the intersection of the horizontal axis H and the major axis of the individual bond point of the first array A14 is about 300.

Array A15 comprises a repeating pattern of oblong shaped bond points 72, such as oval-elliptical, which is preceded and succeeded by a circular/square/diamond shaped bond points 74. In this way, every other bond point in the array has an oblong shape and every other bond point has a circular/square shape.

Similar to array A14, the lengths (e.g., major axes) of the individual oblong shaped bond points of array A15 are typically aligned at an angle that is diagonal relative to the machine direction of the nonwoven fabric. In certain embodiments, the angle formed by the intersection of the horizontal axis H and the major axis of the individual bond points 74 of the array A15 is from about 26° to 34° degrees, and in particular, from about 28° to 32°, and more particularly, 29° to 31°. In a somewhat more preferred embodiment, the angle formed by the intersection of the horizontal axis H and the major axis of the individual bond point of the first array A15 is about 30°.

However, in array A15 the alignment of the lengths (e.g., major axes) of the individual bond points of array A15 are rotated in a directional orientation that is opposite to the alignment of array A14 relative to the vertical axis of the nonwoven fabric. In particular, the lengths (e.g., major axis) of the individual bond points 72 of the array A11 are typically rotated from about 85° to 95°, such as from about 88° to 92°, and more particularly, about 88° to 92° relative to the alignment of the lengths (e.g., major axis) of the individual bond points of array A14.

The average distances d15 between adjacent bond points 74, 72 within the same array of array A15 may range from 1.30 to 1.48 mm, and in particular, from 1.34 to 1.44 mm, with an average distance d15 ranging from about 1.36 to 1.40 mm. Similarly, average distances d16 between adjacent bond points 74 within the same array of array A14 may range 1.30 to 1.48 mm, and in particular, from 1.34 to 1.44 mm, with an average distance d16 ranging from about 1.36 to 1.40 mm.

With reference to FIG. 7D, bonded pattern 78 may further comprise a third array set 82 comprising a pair of alternating arrays extending in a diagonal direction relative to the horizontal and vertical axis of the nonwoven fabric 10c. The third array set 82 comprises a pair of alternating arrays A16, array A17 that are spaced from each other in the machine direction and extend in the diagonal direction of the nonwoven fabric. Each array comprises a plurality of spaced apart individual bond points 72, 74.

Array A16 comprises a repeating pattern of three oblong shaped bond points 72 and a circle/squared shaped bond points 74. In particular, array A16 comprises a first bond point 72a having its major axes aligned the same as the bond points 72 of A10. This is successively followed by second bond point 72b having its major axes aligned the same as the bond points 72 of array A11. Array A11 is followed in succession by third bond point 74c having the same alignment as first bond point 72a. Finally, a circular/square shaped bond 74a point follows in succession third bond point 72c.

The intersection of the array extending in the diagonal direction (DD) (e.g., arrays A16, A17) with the horizontal axis H defines an angle a6. Generally, angle a6 is from about 26° to 34°, and in particular, from about 27° to 33°, more particularly from about 28° to 32°, and even more particularly, from about 29° to 31°. In a preferred embodiment, angle a6 is about 30°.

In certain embodiments, the average collective bond point distance for the embodiments in accordance with FIGS. 7A-7D may ranges from about 1.65 to 1.85 mm. As discussed previously, the average bond point distance is calculated from the average of distances between bond points in the machine, cross, and diagonal direction of the nonwoven fabric. For example, the average collective bond point distance may be calculated from the average distance of d10-d16.

In some embodiments, the bond density (number of individual bond points per square cm) is from about 28 to 38, and in particular, from about 30 to 36, and more particularly, 32 to 34 bond points per cm².

In certain embodiments, the average collective bond point distance is from about 1.70 mm to 1.82 mm, and in particular, from about 1.72 to 1.80 mm, and more particularly, from about 1.74 to 1.75 mm, with an average distance of 1.75 mm being somewhat more preferred.

In certain embodiments in accordance with the embodiments of FIGS. 7A-7D, the bonded nonwoven fabric has an average bond point packing value from about 4.0 to 6.5 mm⁻¹, such as from about 4.5 to 5.75 mm⁻¹, and 4.75 to 5.5 mm⁻¹. In a preferred embodiment, the bonded nonwoven fabric has an average bond point packing value from about 5.0 to 5.25 mm⁻¹.

In certain embodiments, the percentage of bonded surface area of nonwoven fabrics in accordance with embodiments of FIGS. 7A-7D is from about 11 to 12%, the average surface area of the individual bond points is from about 0.25 to 0.5 mm², the bond point packing value is from about 4.5 to 5.5 mm⁻¹, and the bond density is from about 28 to 38 individual point bonds per square centimeter (cm²).

In certain embodiments, the percentage of bonded surface area of nonwoven fabrics in accordance with embodiments of FIGS. 7A-7D is from about 11.4 to 11.6%, the average surface area of the individual bond points is from about 0.3 to 0.4 mm², the bond point packing value is from about 5.05 to 5.15 mm⁻¹, and the bond density is from about 32 to 34 individual point bonds per square centimeter (cm²).

### Exemplary Embodiment D

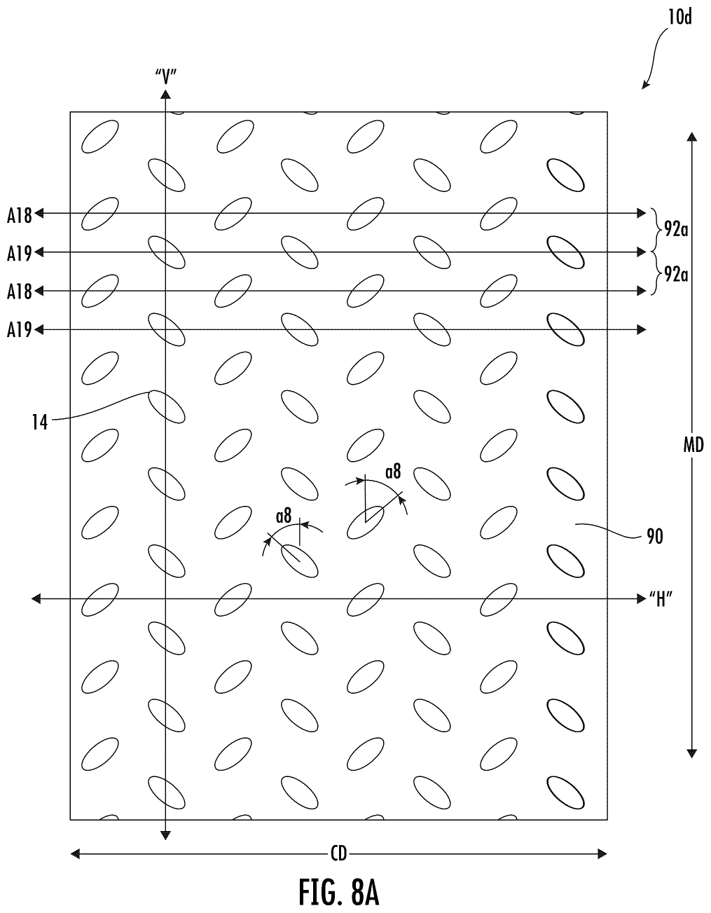

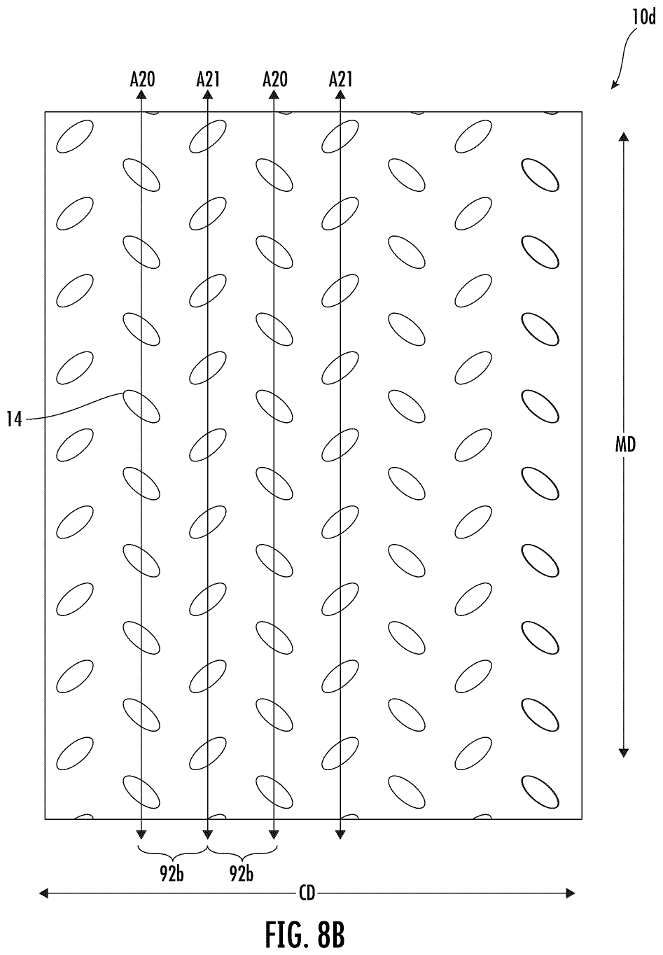

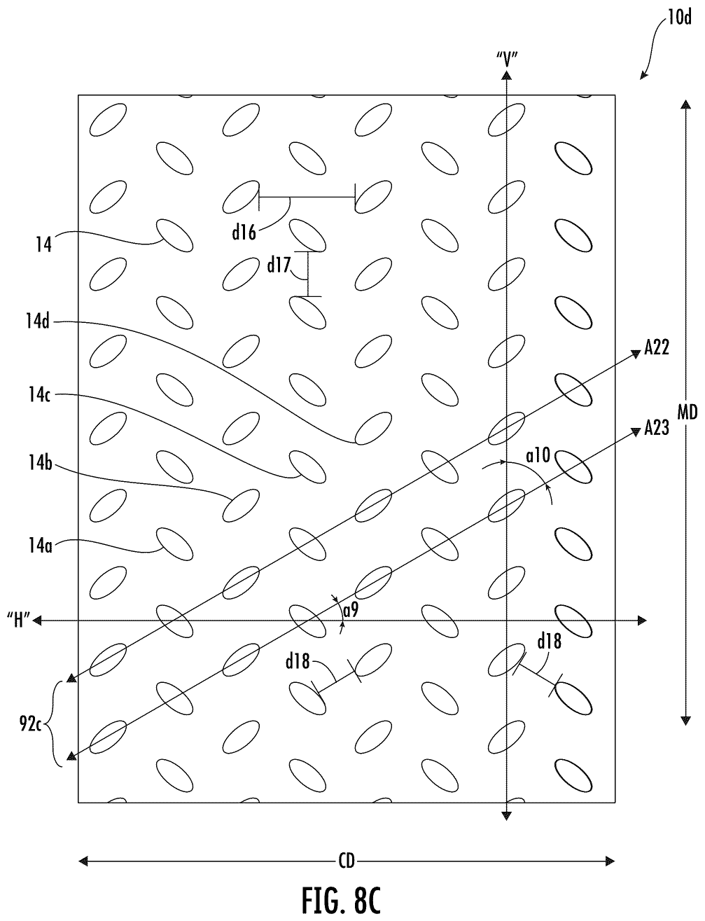

With reference to FIGS. 8A-8C, a further embodiment of the bonded nonwoven fabric is shown and generally indicated by reference character 10d. The nonwoven fabric 10d comprises a plurality of fibers that are point bonded to each other with a plurality of individual bond points 14 to form a coherent web. The nonwoven fabric includes a horizontal axis “H” that is substantially aligned with the cross direction “CD” of the nonwoven fabric and a vertical axis “V” that is substantially aligned with the machine direction “MD” of the nonwoven fabric.

With respect to the nonwowen fabrics in accordance with certain embodiments, the inventors of the present disclosure have discovered that improvements in both abrasion resistance and softness may be obtained with nonwoven fabrics that are thermally point bonded with bond patterns in accordance with one or more embodiments of the present invention. In particular, it has been discovered that a bonded nonwoven fabric having a first bond pattern comprising a plurality of alternating arrays composed of individual bond points in both the machine direction and cross direction of the nonwoven fabric in which the overall percentage of bonded surface area of the nonwoven fabric is less than 14%, the surface area of the individual bond points is from about 0.10 to 0.60 square millimeters (mm²), the bond point packing value is greater than about 2.0 mm⁻¹, such as between 3 to 5 mm⁻¹ and the bond density is from about 20 to 60 individual point bonds per square centimeter (cm²) provides a nonwoven fabric having improved softness and abrasion resistance in comparison to a similar nonwoven fabric having a greater percentage of bonded surface area.

In the embodiment of FIGS. 8A-8C, the overall percentage of bonded surface area of the nonwoven fabric is less than about 14%, the surface area of the individual bond points is from about 0.2 to 0.60 square millimeters (mm²), the bond point packing value is from about 2 to 6 mm⁻¹, and the bond density is from about 28 to 40 individual point bonds per square centimeter (cm²).

The plurality of individual bond points 14 define a first pattern 90 on the surface 12 of the nonwoven fabric 10d. As shown in FIG. 8A, the first pattern 90 comprises a series of alternating first and second arrays A18, A19 of individual bond points 14 that extend in the cross direction of the nonwoven fabric 10d, and that are arranged in array pairs 92a. In certain embodiments, the individual bond points 14 defining the second arrays of the first pattern are offset in the cross direction relative to adjacent bond points of the first arrays. In other words, adjacent bond points of the first and second arrays are not aligned with each other in the machine direction of the nonwoven fabric. This configuration and arrangement can be seen in FIG. 8A, where the vertical axis V only extends through bond points of the second array and does not extend through bond points of the first array. In the embodiment shown in FIG. 8A, the first and second arrays A18, A19 are substantially aligned with the cross direction of the nonwoven fabric 10d.

In a preferred embodiment, the individual bond points of the first arrays do not overlap in the machine direction with the individual bond points of the second arrays. However, it should be recognized that in some embodiments, the individual bond points of the first and second arrays may overlap in the machine direction with bond points in an adjacent array.

In certain embodiments, the percentage of bonded surface area of the nonwoven fabric is from about 13 to 14%, the average surface area of the individual bond points is from about 0.44 to 0.50 mm², the bond point packing value is from about 3 to 5 mm⁻¹, and the bond density is from about 30 to 35 individual point bonds per square centimeter (cm²).

In certain embodiments, the individual bond points have an average length that is from about 1.04 mm to 1.14 mm, and in particular, from about 1.06 to 1.12 mm, and more particularly, from about 1.08 to 1.10 mm. In a preferred embodiment, the individual bond points have an average length that is about 1.09 mm.

In certain embodiments, the individual bond points have an average width that is from about 0.44 to 0.50 mm, and in particular, from about 0.45 to 0.49 mm, and more particularly, from about 0.46 to 0.48 mm. In a preferred embodiment, the individual bond points have an average width that is about 0.47 mm.

In certain embodiments, the average surface area of the individual bond points is from about 0.3 to 0.8 square millimeters (mm²), and in particular, about 0.35 to 0.7 mm², and more particularly, from about 0.38 to 0.5 mm². In some embodiments, the average surface area of the individual bond points is about 0.4 mm².

Referring back to FIG. 8A, the lengths (e.g., major axes) of the individual bond points of the first array are typically aligned at an angle that is diagonal relative to the machine direction of the nonwoven fabric. That is, the intersection of the major axis (see FIG. 2A reference character 30) of individual bond points of the first array and the vertical axis V of the nonwoven fabric defines an angle a8 that is greater than 45° and less than 55°. In certain embodiments, the angle formed by the intersection of the vertical axis V and the major axis of the individual bond point of the first array A18 is from about 48° to 53° degrees, and in particular, from about 49° to 51°, and more particularly, about 50°.

In certain embodiments, the individual bond points of the first array have their lengths (e.g., major axes) aligned in substantially the same direction as each other.

The lengths of the individual bond points of the second array are typically aligned at an angle that is diagonal relative to the machine direction of the nonwoven fabric. That is, the intersection of the major axis 30 of individual bond points of the second array and the horizontal axis H of the nonwoven fabric defines an angle that is greater than 0° and less than 90°. In certain embodiments, the angle formed by the intersection of the horizontal axis H and the major axis of the individual bond point of the second array A19 is from about 43° to 47° degrees, and in particular, from about 44° to 46°, and more particularly, about 45°.

In certain embodiments, the lengths (e.g., major axis) of the individual bond points of the second array are typically rotated from about 88° to 92°, such as from about 89° to 91°, and more particularly, about 90° relative to the alignment of the lengths (e.g., major axis) of the individual bond points of the first array.

In a preferred embodiment, the intersection of a line segment extending along the major axis of individual bond points of the first array and a line segment extending along the major axis of individual bond points of the second array defines an angle that is about 90°.

In certain embodiments, the individual bond points of the second array have their lengths (e.g., major axes) aligned in substantially the same direction as each other.

With reference to FIG. 8B, the first pattern 90 of bond points further comprises a series of alternating third and fourth arrays A20, A21 of individual bond points 14 that extend in the machine direction of the nonwoven fabric 10, and that arranged in array pairs 92b. In the embodiment shown in FIG. 8B, the third and fourth arrays A20, A21 are substantially aligned with the machine direction of the nonwoven fabric 10d. Similar, to the first and second arrays A18, A19, the individual bond points 14 defining the fourth arrays of the first pattern are offset in the machine direction relative to adjacent bond points of the third arrays. In other words, adjacent bond points of the third and fourth arrays are not aligned with each other in the machine direction of the nonwoven fabric.

In certain embodiments, the lengths of the individual bond points of the third array A20 are typically aligned at an angle that is diagonal relative to the vertical axis of the nonwoven fabric. That is, the intersection of the major axis 30 of individual bond points of the third array and the vertical axis V of the nonwoven fabric defines an angle that is greater than 0° and less than 90°. In certain embodiments, the angle formed by the intersection of the horizontal axis V and the major axis of the individual bond point of the third array A20 is from about 43° to 47° degrees, and in particular, from about 44° to 46°, and more particularly, about 45°.

In certain embodiments, the individual bond points of the third array A3 have their lengths (e.g., major axes) aligned in substantially the same direction as each other.

Similarly, the lengths of the individual bond points of the fourth array A21 are typically aligned at an angle that is diagonal relative to the vertical axis of the nonwoven fabric. That is, the intersection of the major axis 30 of individual bond points of the fourth array and the vertical axis V of the nonwoven fabric defines an angle that is greater than 0° and less than 90°. In certain embodiments, the angle formed by the intersection of the vertical axis V and the major axis of the individual bond point of the fourth array A21 is from about 43° to 47° degrees, and in particular, from about 44° to 46°, and more particularly, about 45°.

In certain embodiments, the lengths (i.e., major axis) of the individual bond points of the fourth array are typically rotated from about 88° to 92°, such as from about 89° to 91°, and more particularly, about 90° relative to the alignment of the lengths (i.e., major axis) of the individual bond points of the third array.

In a preferred embodiment, the intersection of a line segment of the major axis of individual bond points of the third array and a line segment of the major axis of individual bond points of the fourth array define an angle that is about 90°.

In certain embodiments, the individual bond points of the fourth array have their lengths (e.g., major axes) aligned in substantially the same direction as each other.

Turning now to FIG. 8C, the first pattern 90 of bond points further comprises a series of alternating fifth and sixth arrays A22, A23 in which the arrays extend diagonally across the surface of the nonwoven fabric relative to the machine direction of the fabric. The fifth and sixth arrays A5, A6 arrays are arranged in array pairs 92c. As shown in FIG. 3C, the intersection of the fifth or sixth arrays with the horizontal axis H defines an angle a9. Generally, angle a9 is from about 25° to 35°, and in particular, from about 27° to 33°, more particularly from about 28° to 32°, and even more particularly, from about 29° to 31°. In a preferred embodiment, angle a9 is about 30°.

Similarly, the intersection of the fifth and sixth arrays with the vertical axis V defines an angle a10. Generally, angle a10 is from about 55° to 65°, and in particular, from about 56° to 34°, more particularly from about 58° to 62°, and even more particularly, from about 59° to 61°. In a preferred embodiment, angle a10 is about 60°.

As shown in FIG. 8C, each successive bond point in the fifth and sixth arrays are rotated approximately 90° relative to the preceding bond point in the same array. In this regard, it can be seen that the lengths of bond points 14a, 14c are aligned substantially in the same direction relative to the horizontal axis H of the nonwoven fabric 10a, and that the lengths of bond points 14b, 14d are rotated approximately 90° relative to the lengths of bond points 14a, 14c. In this way, the major axis of each successive bond point in the same array is substantially perpendicular to the major axis of the preceding or succeeding bond point in the same array.

Continuing to refer to FIG. 8C, the distance d16 between adjacent bond points in the same array in cross direction of the first and second arrays may range from about 2.3 to 2.7 mm, and in particular, from about 2.4 to 2.6, and more particularly, from about 2.48 to 2.52. In a preferred embodiment, the distance d16 between adjacent bond points in the cross direction of the first and second arrays is about 2.5 mm.

In certain embodiments, the distance d17 between adjacent bond points in the machine direction in the same array of the third and fourth arrays may range from about 1.20 to 1.40 mm, and in particular, from about 1.25 to 1.35, and more particularly, from about 1.28 to 1.32 mm. In a preferred embodiment, the distance d17 between adjacent bond points in the same array in the machine direction of the third and fourth arrays is about 1.3 mm.

In certain embodiments, the distance d18 between adjacent bond points in the same arrays of the fifth or sixth arrays (diagonally oriented arrays) may range from about 0.80 to 1.2 mm, and in particular, from about 0.9 to 1.1, and more particularly, from about 0.95 to 1.05. In a preferred embodiment, the distance d18 between adjacent bond points in the same array of the fifth or sixth arrays is about 0.98 to 1.02 mm.

Similarly, for diagonally aligned arrays that are rotated approximately 90° relative to the fifth and sixth arrays (not identified by reference characters), the distance d18 between adjacent bond points in the same array may range from about 0.80 to 1.2 mm, and in particular, from about 0.9 to 1.1, and more particularly, from about 0.95 to 1.05. In a preferred embodiment, the distance d18 between adjacent bond points in the same array of the fifth or sixth arrays is about 0.98 to 1.02 mm.

In certain embodiments, the average collective bond point distance for the embodiments of FIGS. 8A-8C may ranges from about 1.4 to 1.8 mm. As discussed previously, the average bond point distance is calculated from the average of distances between bond points in the machine, cross, and diagonal direction of the nonwoven fabric. For example, the average collective bond point distance may be calculated from the average distance of d16, d17, and d18.

In certain embodiments, the average collective bond point distance is from about 1.45 mm to 1.75 mm, and in particular, from about 1.5 to 1.7 mm, and more particularly, from about 1.55 to 1.65 mm, with an average distance of 1.58 to 1.62 mm being somewhat more preferred.

In certain embodiments in accordance with the embodiments of FIGS. 8A-8C, the bonded nonwoven fabric has an average bond point packing value from about 2 to 6 mm⁻¹, such as from about 2.5 to 5.5 mm⁻¹, and 3.0 to 5.0 mm⁻¹. In a preferred embodiment, the bonded nonwoven fabric has an average bond point packing value from about 3.75 to 4.25 mm⁻¹.

More particularly, in certain embodiments in accordance with the embodiments of FIGS. 8A-8C, the bonded nonwoven fabric has an average bond point packing value greater than 2.0 mm⁻¹, greater than 2.1 mm⁻¹, greater than 2.2 mm⁻¹, greater than 2.3 mm⁻¹, greater than 2.4 mm⁻¹, greater than 2.5 mm⁻¹, greater than 2.6 mm⁻¹, greater than 2.7 mm⁻¹, greater than 2.8 mm⁻¹, greater than 2.9 mm⁻¹, greater than 3.0 mm⁻¹, greater than 3.1 mm⁻¹, greater than 3.2 mm⁻¹, greater than 3.3 mm⁻¹, greater than 3.4 mm⁻¹, greater than 3.5 mm⁻¹, greater than 3.6 mm⁻¹, greater than 3.7 mm⁻¹, greater than 3.8 mm⁻¹, greater than 3.9 mm⁻¹, greater than 4.0 mm⁻¹, greater than 4.1 mm⁻¹, greater than 4.2 mm⁻¹, greater than 4.3 mm⁻¹, greater than 4.4 mm⁻¹, greater than 4.5 mm⁻¹, greater than 4.6 mm⁻¹, greater than 4.7 mm⁻¹, greater than 4.8 mm⁻¹, greater than 4.9 mm⁻¹, greater than 5.0 mm⁻¹, greater than 5.1 mm⁻¹, greater than 5.2 mm⁻¹, greater than 5.3 mm⁻¹, greater than 5.4 mm⁻¹, greater than 5.5 mm⁻¹, greater than 5.6 mm⁻¹, greater than 5.7 mm⁻¹, greater than 5.8 mm⁻¹, greater than 5.9 mm⁻¹, and greater than 6.0 mm⁻¹.

In certain embodiments, in accordance with the embodiments of FIGS. 8A-8C, the bonded nonwoven fabric has an average bond point packing value of less than 6.0 mm⁻¹, less than 5.9 mm⁻¹, less than 5.8 mm⁻¹, less than 5.7 mm⁻¹, less than 5.6 mm⁻¹, less than 5.5 mm⁻¹, less than 5.4 mm⁻¹, less than 5.3 mm⁻¹, less than 5.2 mm⁻¹, less than 5.1 mm⁻¹, less than 5.0 mm⁻¹, less than 4.9 mm⁻¹, less than 4.8 mm⁻¹, less than 4.7 mm⁻¹, less than 4.6 mm⁻¹, less than 4.5 mm⁻¹, less than 4.4 mm⁻¹, less than 4.3 mm⁻¹, less than 4.2 mm⁻¹, less than 4.1 mm⁻¹, less than 4.0 mm⁻¹, less than 3.9 mm⁻¹, less than 3.8 mm⁻¹, less than 3.7 mm⁻¹, less than 3.6 mm⁻¹, less than 3.5 mm⁻¹, less than 3.4 mm⁻¹, less than 3.3 mm⁻¹, less than 3.2 mm⁻¹, less than 3.1 mm⁻¹, less than 3.0 mm⁻¹, less than 2.9 mm⁻¹, less than 2.8 mm⁻¹, less than 2.7 mm⁻¹, less than 2.6 mm⁻¹, less than 2.5 mm⁻¹, less than 2.4 mm⁻¹, less than 2.3 mm⁻¹, less than 2.2 mm⁻¹, less than 2.1 mm⁻¹, and less than 2.0 mm⁻¹

In certain embodiments, the arrays extending in the cross direction of the nonwoven fabric 10d may not be parallel with the horizontal axis H of the nonwoven fabric. As discussed previously with reference to FIG. 4, the plurality of arrays of individual bond points in the cross direction may be non-aligned with the horizontal axis of the nonwoven fabric. As shown in FIG. 4, array A7 defines an array in which the individual bond points 14 extend in the cross direction of the nonwoven fabric at an angle that is greater than 0° relative to the horizontal axis. In particular, the intersection of array (A7 of FIG. 4) and the horizontal axis defines angle a3 that may range from greater than 0° to less than 6°. In a preferred embodiment, angle a3 is from about 0.5° to 4°, and in particular, from about 1° to 3°, with an angle of about 2° being preferred.

As in the embodiments shown in FIGS. 3A-3C, the first pattern defines a plurality of second patterns within the first pattern. Referring back to FIGS. 5A and 5B, the nonwoven fabric 10d may comprise a second pattern 40 comprising individual bond points that collectively define a bond pattern having a quincunx like shape. As in FIGS. 5A and 5B, three adjacent arrays (e.g., A18, A19, A18) of individual bond points further define a plurality of bond patterns in both the machine and cross directions having a quincunx like shape in which four individual bond points defining corners (40a, 40b, 40c, 40d) of the quincunx like bond pattern 40 share a directional orientation that is substantially the same relative to the cross or machine directions of the nonwoven fabric, and wherein an individual bond point 44 defining a center point of the quincunx like bond pattern has a directional orientation that is rotated from about 88° to 92° (e.g., from about 89° to 91°, and in particular, about 90°) relative to the directional orientation of the individual bond points defining the corners of the quincunx like bond pattern. Additional details of the quincunx like bond pattern are discussed previously.

As discussed previously, the inventors of the present disclosure have surprisingly discovered that abrasion resistant is improved in comparison to similarly prepared nonwoven fabrics comprising oval bonds with a higher percentage of bonded area of the nonwoven fabric. In particular, embodiments of the present invention provide improvements in abrasion resistance while having a lower percentage of bonded area in comparison to similar nonwoven fabrics.

The surface area of each individual bond point 14 in accordance with embodiments shown in FIGS. 8A-8C, is typically from about 0.2 to 0.60 mm², and more typically, from about 0.3 to 0.5 mm², and even more typically, about 0.35 to 0.45 mm². In a preferred embodiment, the surface area of each individual bond point is about 0.4 mm².

In certain embodiments, the average surface area of each individual bond point 14 in accordance with embodiments shown in FIGS. 8A-8C, is greater than 0.2, greater than 0.21, greater than, 0.22, greater than 0.23, greater than 0.24, greater than 0.25, greater than 0.26, greater than 0.27, greater than 0.28, greater than 0.29, than 0.30, greater than 0.31, greater than 0.32, greater than 0.33, greater than 0.34, greater than 0.35, greater than 0.36, greater than 0.37, greater than 0.38, greater than 0.39, greater than 0.40, greater than 0.41, greater than, 0.42, greater than 0.43, greater than 0.44, greater than 0.45, greater than 0.46, greater than 0.47, greater than 0.48, greater than 0.49, than 0.50, greater than 0.51, greater than 0.52, greater than 0.53, greater than 0.54, greater than 0.55, greater than 0.56, greater than 0.57, greater than 0.58, greater than 0.59, greater than 0.60 mm².

In certain embodiments, the average surface area of each individual bond point 14 in accordance with embodiments shown in FIGS. 8A-8C, is less than 0.60, less than 0.59, less than, 0.58, less than 0.57, less than, 0.56, less than 0.55, less than, 0.55, less than 0.53, less than, 0.52, less than 0.51, less than, 0.50, less than 0.49, less than, 0.48, less than 0.47, less than, 0.46, less than 0.45, less than, 0.44, less than 0.43, less than, 0.42, less than 0.41, less than, 0.40, less than 0.39, less than, 0.38, less than 0.37, less than, 0.36, less than 0.35, less than, 0.34, less than 0.33, less than, 0.32, less than 0.31, less than, 0.30, less than 0.29, less than, 0.28, less than 0.27, less than, 0.26, less than 0.25, less than, 0.24, less than 0.23, less than, 0.22, less than 0.21, less than, 0.20 mm².

In certain embodiments of the invention, the number of individual bond points per cm² is from about 30 to 36, and in particular, from about 32.5 to 34.5, and more particularly, from about 33 to 24.

In some embodiments, the percent bonded area of the nonwoven fabric 10d is from about 13 to 14%, and in particular, from about 13.1 to 13.9%, and more particularly, from about 13.2 to 13.8%. In a preferred embodiment, the percent bonded area of the nonwoven fabric from about 13.3 to 13.5%.

The bonding patterns described herein may be used to thermally bond a variety of different nonwoven structures.

In one embodiment, the present disclosure provides a spunbond nonwoven fabric comprising a plurality of fibers that are thermally point bonded to each other to form a coherent web.

Although, the present invention is generally discussed in the context of spunbond fabrics prepared from continuous filaments, it should be recognized that other nonwoven fabrics and fibers may be prepared in accordance with embodiments of the invention including meltblown fibers and meltblown fabrics, staple fibers and carded fabric, wet-laid fabrics, resin bonded fabrics, and air-laid fabrics, and combinations thereof.

In certain embodiments, the fibers of the nonwoven fabric may comprise monocomponent fibers, multicomponent fibers, or combinations thereof.

In one embodiment, the fibers of the nonwoven fabric comprise multicomponent fibers that may include at least two polymer components arranged in structured domains across the cross section of the fiber. As is generally well known to those skilled in the art, polymer domains or components are arranged in substantially continuously positioned zones across the cross-section of the multicomponent fiber and extend continuously along the length of the multicomponent fiber. More than two components could be present in the multicomponent fiber.

A preferred configuration is a side-by-side arrangement wherein a first polymer component defines a first continuous distinct zone extending along the length of the fiber, and a second polymer component defines a second continuous distinct zone extending along the length of the fiber. Both the first and second polymer components define at least a portion of the outer surface of the continuous fibers. In certain embodiments, the first and second distinct zones of the side-by-side continuous fibers are present in ratios ranging from 10:90 to 90:10, and in particular, from about 40:60 to 60:40, and more particularly, from about 50:50. Side-by-side configurations are particularly useful in the preparation of crimped fibers. Other configurations that may be useful in the preparation of crimped fibers include eccentric sheath/core and D-centric sheath core configurations.

Another preferred configuration is a sheath/core arrangement wherein a first component, the sheath, substantially surrounds a second component, the core. The resulting sheath/core bicomponent fiber may have a round or non-round cross-section. Other structured fiber configurations as known in the art can be used including, segmented pie, islands-in-the-sea and tipped multilobal structures.

In certain embodiments, the fibers are bicomponent in which a first polymer component defines a sheath of the fiber, and a second polymer component defines a core of the fiber. Generally, the weight percentage of the sheath to that of the core in the fibers may vary widely depending upon the desired properties of the nonwoven fabric. For example the weight ratio of the sheath to the core may vary between about 5:95 to 95:5, such as from about 10:90 to 90:10, and in particular from about 20:80 to 80:20. In a preferred embodiment, the weight ratio of the sheath to the core is about 25:75 to 35:65, with a weight ratio of about 30:70 to 50:50 being preferred.

Preferred sheath/core bicomponent fibers for use in making fabrics of this invention can have the higher melting component as the core and the lower melting component as the sheath. For example, an aliphatic polyester component could be used as the sheath and the core could be a higher melting polymer component comprising a polyolefin, such as polypropylene. Such a structure with an aliphatic polyester on the surface allows use of a reduced calender oil bonding temperature thus conserving energy during manufacture of the nonwoven web.

A wide variety of polymers may be used in the preparation of nonwoven fabrics in accordance with embodiments of the present disclosure.

Nonwoven fabrics in accordance with embodiments of the invention may be prepared with a wide variety of different polymers and polymeric blends. Examples of suitable polymers for preparing the fibers include polyolefins, such as polypropylene and polyethylene, and copolymers thereof, polyesters, such as polyethylene terephthalate (PET), polytrimethylene terephthalate (PTT), and polybutylene terephthalate (PBT), nylons, polystyrenes, polyurethanes, copolymers, and blends thereof, and other synthetic polymers that may be used in the preparation of fibers. In some embodiment, the polymer can be selected from the group consisting of: polyolefins, polyesters, polyethylene terephthalates, polybutylene terephthalates, polycyclohexylene dimethylene terephthalates, polytrimethylene terephthalates, polymethyl methacrylates, polyamides, nylons, polyacrylics, polystyrenes, polyvinyls, polytetrafluoroethylenes, ultrahigh molecular weight polyethylenes, very high molecular weight polyethylenes, high molecular weight polyethylenes, polyether ether ketones, non-fibrous plasticized celluloses, polyethylenes, polypropylenes, polybutylenes, polymethylpentenes, low-density polyethylenes, linear low-density polyethylenes, high-density polyethylenes, polystyrenes, acrylonitrile-butadiene-styrenes, styrene-acrylonitriles, styrene tri-block and styrene tetra block copolymers, styrene-butadienes, styrene-maleic anhydrides, ethylene vinyl acetates, ethylene vinyl alcohols, polyvinyl chlorides, cellulose acetates, cellulose acetate butyrates, plasticized cellulosics, cellulose propionates, ethyl cellulose, natural fibers, any derivative thereof, any polymer blend thereof, any copolymer thereof or any combination thereof.

In certain embodiments, the polymers for use in the fibers preferably comprise a polyolefin, such as a polypropylene, polyethylene, a blend of a polypropylene and polyethylene, and combinations thereof.

A wide variety of polypropylenes may be used in embodiments of the invention typically have molecular weights greater than about 120,000 g/mol, and more typically, may have molecular weights ranging from about 150,000 to about 300,000 g/mol. In one embodiment, the polypropylene may have a molecular weights ranging from about 160,000 to about 250,000 g/mol, and in particular, from about 160,000 to about 180,000 g/mol.

In certain embodiments for the preparation of spunbond fibers, polypropylenes that may be used have an MFR that is typically from about 10 to 100 g/10 min, and in particular, from about 20 to 40 g/10 min, with an MFR from about 22 to 38 g/10 min being somewhat more typical. Unless otherwise indicated MFR is measured in accordance with ASTM D-1238.

Examples of such polypropylenes may include those available from ExxonMobil, such as PP3155 (36 MFR g/10 min, density of 0.90 g/cm³, and Mw 172 k g/mol); PP3155E5 (36 MFR g/10 min, density of 0.90 g/cm³, and Mw 172 k g/mol); and ACHIEVE™ 3854 (24 MFR g/10 min, density of 0.90 g/cm³). Polypropylenes available from SABIC®, such as SABIC PP 511A (25 MFR g/10 min, density of 0.905 g/cm³), polypropylenes available from Borealis, such as HG475FB (27 MFR g/10 min), and polypropylenes available from Braskem, such as CP360H (34 MFR g/10 min) may also be used.

In certain embodiments for the preparation of meltblown fibers, polypropylenes that may be used have an MFR that is typically greater than about 500 g/10 min may be used. For example, the polypropylene may have an MFR from about 500 to 2500 g/10 min, and in particular, from about 1000 to 1500 g/10 min, with an MFR from about 1200 to 1400 g/10 min being somewhat more typical. An example of such a polypropylene is available from Braskem, such as H155 (1284 MFR) g/10 min.

In certain embodiments, the fibers may comprise a multicomponent fiber, such as a bicomponent fiber comprising a first polymer component and a second polymer component in which the second polymer component comprises a blend of polyolefins in which a first polyolefin in the blend has a low MFR, such as less than 100 g/10 min and the second polyolefin in the blend has an MFR greater than the first polyolefin, such as greater than 500 g/10 min, and in particular, greater than 1,000 g/10 min. Typically, the MFR of the blend is less than 50 g/10 min and the MFR ratio of the low MFR polyolefin to the high MFR polyolefin is 1:100, and in particular: 1:20 to 1:50. Typically, the amount of high MFR in the blend is from about 0.5 to 12 weight percent, based on the total weight of the blend, and in particular, from about 2 to 8, weight percent, and more particularly, from about 3 to 6 weight percent, based on the total weight of the blend.

In one such embodiment, the first polymer component comprises a polypropylene polymer having an MFR from about 20 to 40 g/10 min, and the second polymer component comprises a blend of a low MFR polypropylene having an MFR from about 20 to 40 g/10 min and a high MFR polypropylene having an MFR from about 1,100 to 1,400 g/10 min in which the amount of high MFR polypropylene in the blend is from about 3 to 6 weight percent, based on the total weight of the blend. The polypropylene in the first polymer component may be the same or a different polypropylene as the low MFR polypropylene in the second polymer component. Such fibers when prepared in a side-by-side, eccentric or D-centric configuration may be used to prepare a nonwoven fabric comprising crimped fibers.

In some embodiments, the polyolefin may comprise a polyethylene polymer. Various types of polyethylene polymers may be employed in the fibers of the present invention. As an example, a high density polyethylene, a branched (i.e., non-linear) low density polyethylene, or a linear low density polyethylene (LLDPE) can be utilized. Polyethylenes may be produced from any of the well-known processes, including metallocene and Ziegler-Natta catalyst systems. Generally, the polyethylene polymers that are conventionally used in the production of spunbond fabrics may be suitable for use in the present invention.

In one embodiment of the invention, the polyethylene component comprises a polyethylene having a density ranging from about 0.90 to 0.97 g/cm³ (ASTM D-792). In particular, preferred polyethyelenes have a density value ranging from 0.93 to 0.965 g/cm³, and more particularly from about 0.94 to 0.965 g/cm³. Examples of suitable polyethylenes included ASPUN™ 6834 (a polyethylene polymer resin having a melt index of 17 g/10 min (ISO 1133) and a density of 0.95 g/cm³ (ASTM D-792)), available from Dow Chemical Company, and HD6908.19 (a polyethylene resin supplied by ExxonMobil having a melt index in the range of 7.5 to 9 g/10 min (ISO 1133) and a density of 0.9610 to 0.9680 g/cm³ (ASTM D-792)).

LLDPE may also be used in some embodiments of the present invention. LLDPE is typically produced by a catalytic solution or fluid bed process under conditions established in the art. The resulting polymers are characterized by an essentially linear backbone. Density is controlled by the level of comonomer incorporated into the otherwise linear polymer backbone. Various alpha-olefins are typically copolymerized with ethylene in producing LLDPE. The alpha-olefins which preferably have four to eight carbon atoms, are present in the polymer in an amount up to about 10 percent by weight. The most typical comonomers are butene, hexene, 4-methyl-1-pentene, and octene. In general, LLDPE can be produced such that various density and melt index properties are obtained which make the polymer well suited for melt-spinning with polypropylene. Preferably, the LLDPE should have a melt index of greater than 10, and more preferably 15 or greater for spunbonded filaments. Particularly preferred are LLDPE polymers having a density of 0.90 to 0.97 g/cm³ and a melt index of greater than 25. Examples of suitable commercially available linear low density polyethylene polymers include those available from Dow Chemical Company, such as ASPUN™ Type 6811 (27 MFR g/10 min, density 0.923 g/cm³), ASPUN™ Type 6834 (17 MFR g/10 min, density of 0.95 g/cm³), ASPUN™ Type 6000 (30 MFR g/10 min, 0.955 g/cm³ density), ASPUN™ Type 6850 (30 MFR g/10 min, 0.955 g/cm³ density), Dow LLDPE 2500 (55 MFR g/10 min, 0.923 g/cm³ density), Dow LLDPE Type 6808A (36 MFR g/10 min, 0.940 g/cm³ density), and the Exact series of linear low density polyethylene polymers from Exxon Chemical Company, such as Exact 2003 (31 MFR g/10 min, density 0.921 g/cm³).

In some embodiments, the polymers may be extensible and/or elastic.

In certain embodiments, the nonwoven fabric comprises a blend of an olefin polymer and elastomeric olefin copolymer.

In applications directed to preparation of spunbond fabrics, the olefin polymer typically has an MFR from about 5 to 150 g/10 min, with an MFR from about 15 to 50 g/10 min, and more particularly, an MFR from about 20 to 40 g/10 min being somewhat more preferred. The amount of the olefin polymer in the fibers is typically from 75 to 95 weight percent, based on the total weight of the fiber, and in particular, from about 80 to 95 weight percent, based on the total weight of the fiber.

In addition, polypropylenes that may be used as the first polypropylene may have an MFR that is from about 10 to 100 g/10 min, and in particular, from about 20 to 40 g/10 min, with an MFR from about 22 to 38 g/10 min being somewhat more typical. Unless otherwise indicated MFR is measured in accordance with ASTM D-1238.

Examples of such polypropylenes may include those available from ExxonMobil, such as PP3155 (36 MFR g/10 min, density of 0.90 g/cm³, and Mw 172 k g/mol); PP3155E5 (36 MFR g/10 min, density of 0.90 g/cm³, and Mw 172 k g/mol); and ACHIEVE™ 3854 (24 MFR g/10 min, density of 0.90 g/cm³). Polypropylenes available from SABIC®, such as SABIC PP 511A (25 MFR g/10 min, density of 0.905 g/cm³), and polypropylenes available from Borealis, such as HG475FB (27 MFR g/10 min) may also be used.

In a preferred embodiment, the olefin polymer comprises polypropylene. A wide variety of polypropylenes may be used as the olefin polymer in the fibers. Suitable polypropylenes may be produced from any of the well-known processes, including metallocene and Ziegler-Natta catalyst systems.

In certain embodiments, the olefin copolymer comprises a propylene, copolymer comprising at least two different types of monomer units, one of which is propylene. Suitable examples of monomer units include, for example, ethylene and higher α-olefins in the range of C₄ to C₂₀, such as 1-butene, 4-methyl-1-pentene, 1-hexene, or 1-octene. And 1-decene, or mixtures thereof, Preferably, ethylene is copolymerized with propylene, so that the propylene copolymer comprises propylene units (polymer chain units derived from propylene monomers) and ethylene units (polymer chain units derived from ethylene monomers).

The olefin copolymer is present as a minor component in the polypropylene blend. The amount of the olefin copolymer in the blend is typically from 5 to 25 weight percent, based on the total weight of the fiber, and in particular, from about 6 to 20 weight percent, based on the total weight of the fiber. More particularly, the amount of the olefin compolymer in the blend is from about 1 to 25 weight percent, based on the total weight of the blend. In particular, the amount of the olefin copolymer may be from about 2 to 20 weight percent, such as from about 4 to 16 weight percent, from about 5 to 15 weight percent, and from about 6 to 14 weight percent, based on the total weight of the blend.

Typically, the units or comonomers of the propylene copolymer are derived from ethylene or at least one of C4-10 alpha-olefins are from 1% to 35%, or from 5% to about 35% by weight of the propylene-alpha-olefin copolymer. It may be present in an amount of wt %, or 7 wt % to 32 wt %, or 8 to about 25 wt %, or 8 wt % to 20 wt %, or even 8 wt % to 18 wt %. The comonomer content is such that the propylene-α-olefin copolymer preferably has an isothermal heat of fusion (“DSC”) of 75000 Gy (75 J/g) or less, a melting point of 100° C., or less, and a crystallinity of 2% to about 65%. It has tactic polypropylene and can preferably be adjusted to have a melt flow rate of 0.5 to 90 dg/min.

In certain embodiments, the propylene-α-olefin copolymer may consist of ethylene derived units. The propylene-α-olefin copolymer is 5% to 35%, or 5% to 20%, or 10% to 12%, or 15% to 20% by weight of the propylene-α-olefin copolymer. It may contain weight percent ethylene derived units. In some embodiments, the propylene-α-olefin copolymer consists essentially of units derived from propylene and ethylene, ie, the propylene-α-olefin copolymer is ethylene and/or propylene used during polymerization.

In certain embodiments, the propylene-α-olefin copolymer may have a triad tacticity of three propylene units (measured by 13C NMR) of at least 75%, at least 80%, at least 82%., at least 85%, or at least 90%. “Triad tacticity” is determined as follows. The tacticity ratio (denoted herein as “m/r”) is determined by 13C nuclear magnetic resonance (“NMR”). The tacticity rate ni/r N. Calculated by Cheng as defined in 17 MACROMOLECULES 1950 (1984), which is incorporated herein by reference. The notation “m” or “r” represents the stereochemistry of a pair of adjacent propylene groups, “m” refers to meso, and “r” refers to racemic. An m/r of 1.0 generally represents a syndiotactic polymer and an in/r ratio of 2.0 generally represents an atactic material. Isotactic materials theoretically have m/r ratios approaching infinity, and many byproduct atactic polymers have sufficient isotactic content to produce m/r ratios greater than 50.

Examples of suitable propylene-α-olefin copolymers may include VISTAMAXX® (ExxonMobil Chemical Company, Houston, Tex., USA), VERSIFY® (The Dow Chemical Company, Midland, Mich., USA), Grades of TAFMER® XM or NOTIO® (Mitsui Company, Japan), and grades of SOFTEL® (Basell Polyfins of the Netherlands).

In certain embodiments, the nonwoven fabric may comprise fibers comprising a blend of a first polypropylene polymer and an olefin copolymer in which the olefin copolymer comprises a low isotacticity polypropylene (e.g., a polypropylene having an isotacticity [mmmm]from 30 to 70% by mol).

Accordingly, in certain embodiments the low isotacticity polypropylene may be present in an amounts from about 1 to 25 weight percent, 2 to 24 weight percent, 3 to 22 weight percent, 4 to 21 weight percent, 5 to 20 weight percent, 6 to 19 weight percent, 7 to 18 weight percent, 8 to 17 weight percent, 9 to 16 weight percent, and 10 to 15 weight percent, based on the total weight of the first polypropylene component.

The low isotacticity polypropylene may generally be characterized by one or more of the following properties:

isotacticity: a meso pentad fraction [mmmm] of 20 to 70% by mol;
average number molecular weight (Mw) of 10,000 to 200,000;
a melting temperature from about 60 to 120° C.; and
a melt flow rate (MFR) greater than 40 g/10 min.

In addition to the above properties the low isotacticity polypropylene may have a B-viscosity from about 7,000 to 400,000 mPa, and a tensile modulus from about 80 to 120 MPa.

Low isotacticity polypropylenes polymers that are suitable generally have an isotacticity [mmmm] (% by mol) that is between about 20 and 70, and in particular, a [mmmm] between 30 and 60% by mol, and more particularly, a [mmmm] between 35 and 55% by mol. In one embodiment, the low isotacticity polypropylene has an isotacticity [mmmm] that is between about 40 and 50% by mol.

The stereochemistry (e.g., stereoregularity index ([mm]), meso pentad fraction [mmmm], the racemic pentad fraction [rrrr], the racemic-meso-racemic-meso pentad fraction [rmrm], and triad fractions [mm] [rr] and [mr]) of the low isotacticity polypropylene may be determined with an ¹³C-NMR spectrum according to the attribution of peaks proposed by A. Zambelli, et al., Macromolecules, No. 8, p. 687 (1975). A ¹³C-NMR, Model JNM-EX400, produced by JEOL Ltd. may be used to obtain the spectrum according to the following parameters:
Method: proton complete decoupling method;
Concentration: 220 mg/mL;
Solvent: mixed solvent of 1,2,4-trichlorobenzene and deuterated benzene (90/10 by volume);
Temperature: 130° C.;
Pulse width: 450;
Pulse repetition time: 4 sec;
Accumulation: 10,000 times;

$$M = m / S \times 100 \\ R = γ / S \times 100 \\ S = P ββ + P α β + P α γ \\ S = P ββ : 19.8 - 22.5 ppm \\ P αβ : 18. - 17.5 ppm \\ P α γ : 17.5 - 17.1 ppm \\ γ : racemic pentad chain \text{: 2} 0.7 - 20.3 ppm \\ m : meso pentad chain : 21.7 - 22.5 ppm . \qquad < Calculation Expression >$$

In one embodiment, the low isotactic polypropylene has an isotacticity [mmmm] (% by mol) that is greater than about 30, greater than about 31, greater than about 32, greater than about 33, greater than about 34, greater than about 35, greater than about 36, greater than about 37 greater than about 38, greater than about 39, greater than about 40, greater than about 41, greater than about 42, greater than about 43, greater than about 44, greater than about 45, greater than about 45, greater than about 47, greater than about 48, greater than about 49, greater than about 50, greater than about 51, greater than about 52, greater than about 53, greater than about 54, greater than about 55, greater than about 56, greater than about 57, greater than about 58, greater than about 59, and greater than about 60.

In one embodiment, the low isotacticity polypropylene has an isotacticity [mmmm](% by mol) that is less than about 60, less than about 59, less than about 58, less than about 57, less than about 56, less than about 55, less than about 54, less than about 53, less than about 52, less than about 51, less than about 50, less than about 49, less than about 48, less than about 47, less than about 46, less than about 45, less than about 44, less than about 43, less than about 42, less than about 41, less than about 40, less than about 39, less than about 38, less than about 37, less than about 36, less than about 35, less than about 34, less than about 33, less than about 32, and less than about 31.

In some embodiments, the low isotacticity polypropylene may have a crystallinity that is from about 30 to 60 percent, such as between 35 and 55 percent, between 40, and 50 percent, and preferably, between 42 and 48 percent. In one embodiment, the low isotacticity polypropylene may have a crystallinity that is from about 44 to 46 percent. Crystallinity of the low isotacticity polypropylene may be measured in accordance with ASTM D-3418-15.

In one embodiment, the low isotacticity polypropylene typically has an MFR greater than 40 g/10 min and a molecular weight of less than 140,000 g/mol, and in particular, an MFR greater than 45 g/10 min and a molecular weight less than 135,000 g/mol. In a preferred embodiment, the low isotacticity polypropylene has a molecular weight between 125,000 g/mol and 135,000 g/mol and an MFR from about 45 to 55 g/10 min. Unless otherwise indicated MFR is measured in accordance with ASTM D-1238.

In certain embodiments, the low isotacticity polypropylene has a melting temperature that is greater than about 60° C., and in particular, from about 60 to 120° C., and more particularly, from about 60 to 100° C. In one embodiment, the low isotacticity polypropylene has a melting temperature that is from about 65 to 85° C., and in particular, from about 70 to 80° C. The melting temperature of low isotacticity polypropylene can be determined in accordance with ISO 306 Method A50.

In certain embodiments, the low tacticity polypropylene has a molecular weight ranging from about 30,000 to about 150,000 g/mol, and in particular, from about 45,000 to about 140,000 g/mol, and more particularly, from about 70,000 to 135,000 g/mol. In a preferred embodiment, the low isotacticity polypropylene has a molecular weight that is from about 128,000 to about 132,000 g/mol.

In one embodiment, the low isotacticity polypropylene may have a molecular weight less than one of the following: less than about 150,000 g/mol, less than about 145,000 g/mol, less than about 140,000 g/mol, less than about 138,000 g/mol, less than about 136,000 g/mol, less than about 134,000 g/mol, less than about 132,000 g/mol, less than about 130,000 g/mol, less than about 128,000 g/mol, less than about 126,000 g/mol, less than about 124,000 g/mol, less than about 122,000 g/mol, less than about 120,000 g/mol, less than about 118,000 g/mol, less than about 116,000 g/mol, less than about 114,000 g/mol, less than about 112,000 g/mol, less than about 110,000 g/mol, less than about 108,000 g/mol, less than about 106,000 g/mol, less than about 104,000 g/mol, less than about 102,000 g/mol, less than about 100,000 g/mol, less than about 98,000 g/mol, less than about 96,000 g/mol, less than about 94,000 g/mol, less than about 92,000 g/mol, less than about 90,000 g/mol, less than about 88,000 g/mol, less than about 86,000 g/mol, less than about 84,000 g/mol, less than about 82,000 g/mol, less than about 80,000 g/mol, less than about 78,000 g/mol, less than about 76,000 g/mol, less than about 74,000 g/mol, less than about 72,000 g/mol, or less than about 70,000 g/mol.

In some embodiments, the low isotacticity polypropylene has a molecular weight that is less than the molecular weight of the first polypropylene in which it is blended. For example, in certain embodiments of the present invention, the percent difference in the molecular weight between the first polypropylene and the low isotacticity polypropylene is from 5 to 150% In one embodiment, the percent difference may be between 7 and 120%. In a preferred embodiment, the percent difference in the molecular weight between the first polypropylene and the low isotacticity polypropylene is from about 20 to 35%, and more preferably, from about 25 to 30%.

In the context of the present invention, percent difference is calculated according to the following:

$$PERCENT DIFFERENCE = \frac{\vert {V}_{1} - {V}_{2} \vert}{[ \frac{( {V}_{1} + {V}_{2} )}{2} ]} \times 100$$

In one embodiment, the first polypropylene has a molecular weight of 172,000 g/mol and the low isotacticity weight polypropylene has a molecular weight that is about 130,000 to provide a percent difference of about 27.8%. In another embodiment, the first polypropylene may have a molecular weight of about 140,000 g/mol, and the low isotacticity polypropylene may have a molecular weight of 130,000 g/mol to provide a percent difference of about 7%. In a further embodiment, the first polypropylene may have a molecular weight of about 172,000 g/mol, and the low isotacticity polypropylene may have a molecular weight of 45,000 to provide a percent difference of about 117%

Examples of suitable low isotacticity polypropylenes are available from Idemitsu under the product name L-MODUT™. Examples include S400 (˜2,600 MFR g/10 min, density of 0.87 g/cm³, and M_{w} 45 k g/mol); S600 (390 MFR g/10 min, density of 0.87 g/cm³, and M_{w} 75 k g/mol); and S901 (50 MFR g/10 min, density of 0.87 g/cm³, and M_{w} 130 k g/mol).

In other embodiments, the low isotacticity polypropylene may comprise a copolymer of ethylene and propylene units.

As discussed above, the low isotacticity polypropylene is blended with the first polypropylene. Typically, the blending occurs in an extruder under heat and pressure to produce a homogeneous blend prior to be introduced into the spin beam as a molten or semi-molten polymer stream.

The amount of the low isotacticity polypropylene in the blend is typically from about 0.1 to 40 weight percent, based on the total weight of the polypropylene component, and in particular, from about 5 to 25 weight percent, based on the total weight of the polypropylene component. In one embodiment, the amount of the low isotacticity polypropylene in the blend is typically from about 5 to 20 weight percent, and more typically, from about 8 to 16 weight percent, and even more typically, from about 10 to 15 weight percent, based on the total weight of the propylene component.

In some embodiments, the polypropylene component may include additives, such as pigments, antimicrobial agents, processing aids, fillers, such as Ca₂O₃, hydrophilic agents, antistatic agents, hydrophobic additives, botanicals, such as aloe vera, vitamin E, flame retardants, biodegradeable enhancement agents, slip agents, and the like.

In some embodiments, the polymers may comprise polymers derived from mechanically or chemically recycled feedstocks. For example, up to 100% of the polymer comprising the nonwoven fabric may be derived from recycled polymers.

In further embodiments, nonwoven fabrics nonwoven fabrics in accordance with one or more embodiments of the invention may be prepared from bio-based materials, and in particular, from bio-based polymers. In contrast to polymers derived from petroleum sources, bio-based polymers are generally derived from a bio-based material. In some embodiments, a bio-based polymer may also be considered biodegradeable. A special class of biodegradable product made with a bio-based material might be considered as compostable if it can be degraded in a composting environment. The European standard EN 13432, “Proof of Compostability of Plastic Products” may be used to determine if a fabric or film comprised of sustainable content could be classified as compostable.

In one such embodiment, the nonwoven fabric comprises fibers comprising a bio-based polymer. In certain embodiments, the fibers are substantially free of synthetic materials, such as petroleum-based materials and polymers. For example, fibers comprising the nonwoven fabric may have less than 25 weight percent of materials that are non-bio-based, and more preferably, less than 20 weight percent, less than 15 weight percent, less than 10 weight percent, and even more preferably, less than 5 weight percent of non-bio-based materials, based on the total weight of the nonwoven fabric.

In certain embodiments, the nonwoven fabric may comprise fibers comprising a bio-based polymer and a polymer derived from a petroleum source.

In one embodiment, bio-based polymers for use may include aliphatic polyester based polymers, such as polylactic acid, and bio-based derived polyethylene.

Aliphatic polyesters useful in the present invention may include homo- and copolymers of poly(hydroxyalkanoates), and homo- and copolymers of those aliphatic polyesters derived from the reaction product of one or more polyols with one or more polycarboxylic acids that are typically formed from the reaction product of one or more alkanediols with one or more alkanedicarboxylic acids (or acyl derivatives). Polyesters may further be derived from multifunctional polyols, e.g. glycerin, sorbitol, pentaerythritol, and combinations thereof, to form branched, star, and graft homo- and copolymers. Polyhydroxyalkanoates generally are formed from hydroxyacid monomeric units or derivatives thereof. These include, for example, polylactic acid, polyhydroxybutyrate, polyhydroxyvalerate, polycaprolactone and the like. Miscible and immiscible blends of aliphatic polyesters with one or more additional semicrystalline or amorphous polymers may also be used.

One useful class of aliphatic polyesters are poly(hydroxyalkanoates), derived by condensation or ring-opening polymerization of hydroxy acids, or derivatives thereof. Suitable poly(hydroxyalkanoates) may be represented by the formula: H(O—R—C(O)—)ₙOH where R is an alkylene moiety that may be linear or branched having 1 to 20 carbon atoms, preferably 1 to 12 carbon atoms optionally substituted by catenary (bonded to carbon atoms in a carbon chain) oxygen atoms; n is a number such that the ester is polymeric, and is preferably a number such that the molecular weight of the aliphatic polyester is at least 10,000, preferably at least 30,000, and most preferably at least 50,000 daltons. In certain embodiments, the molecular weight of the aliphatic polyester is typically less than 1,000,000, preferably less than 500,000, and most preferably less than 300,000 daltons. R may further comprise one or more caternary (i.e. in chain) ether oxygen atoms. Generally, the R group of the hydroxy acid is such that the pendant hydroxyl group is a primary or secondary hydroxyl group.

Useful poly(hydroxyalkanoates) include, for example, homo- and copolymers of poly(3-hydroxybutyrate), poly(4-hydroxybutyrate), poly(3-hydroxyvalerate), poly(lactic acid) (as known as polylactide), poly(3-hydroxypropanoate), poly(4-hydropentanoate), poly(3-hydroxypentanoate), poly(3-hydroxyhexanoate), poly(3-hydroxyheptanoate), poly(3-hydroxyoctanoate), polydioxanone, polycaprolactone, and polyglycolic acid (i.e. polyglycolide). Copolymers of two or more of the above hydroxy acids may also be used, for example, poly(3-hydroxybutyrate-co-3-hydroxyvalerate), poly(lactate-co-3-hydroxypropanoate), poly(glycolide-co-p-dioxanone), and poly(lactic acid-co-glycolic acid). Blends of two or more of the poly(hydroxyalkanoates) may also be used, as well as blends with one or more semicrystalline or amorphous polymers and/or copolymers.

The aliphatic polyester may be a block copolymer of poly(lactic acid-co-glycolic acid). Aliphatic polyesters useful in the inventive compositions may include homopolymers, random copolymers, block copolymers, star-branched random copolymers, star-branched block copolymers, dendritic copolymers, hyperbranched copolymers, graft copolymers, and combinations thereof.

Another useful class of aliphatic polyesters includes those aliphatic polyesters derived from the reaction product of one or more alkanediols with one or more alkanedicarboxylic acids (or acyl derivatives). Such polyesters have the general formula:

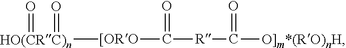

where R′ and R″ each represent an alkylene moiety that may be linear or branched having from 1 to 20 carbon atoms, preferably 1 to 12 carbon atoms, and m is a number such that the ester is polymeric, and is preferably a number such that the molecular weight of the aliphatic polyester is at least 10,000, preferably at least 30,000, and most preferably at least 50,000 daltons, but less than 1,000,000, preferably less than 500,000 and most preferably less than 300,000 daltons. Each n is independently 0 or 1. R′ and R″ may further comprise one or more caternary (i.e. in chain) ether oxygen atoms.

Examples of aliphatic polyesters include those homo- and copolymers derived from (a) one or more of the following diacids (or derivative thereof): succinic acid; adipic acid; 1,12 dicarboxydodecane; fumaric acid; glutartic acid; diglycolic acid; and maleic acid; and (b) one of more of the following diols: ethylene glycol; polyethylene glycol; 1,2-propane diol; 1,3-propanediol; 1,2-propanediol; 1,2-butanediol; 1,3-butanediol; 1,4-butanediol; 2,3-butanediol; 1,6-hexanediol; 1,2 alkane diols having 5 to 12 carbon atoms; diethylene glycol; polyethylene glycols having a molecular weight of 300 to 10,000 daltons, and preferably 400 to 8,000 daltons; propylene glycols having a molecular weight of 300 to 4000 daltons; block or random copolymers derived from ethylene oxide, propylene oxide, or butylene oxide; dipropylene glycol; and polypropylene glycol, and (c) optionally a small amount, i.e., 0.5-7.0 mole percent of a polyol with a functionality greater than two, such as glycerol, neopentyl glycol, and pentaerythritol.

Such polymers may include polybutylene succinate homopolymer, polybutylene adipate homopolymer, polybutyleneadipate-succinate copolymer, polyethylenesuccinate-adipate copolymer, polyethylene glycol succinate homopolymer and polyethylene adipate homopolymer.

Commercially available aliphatic polyesters include poly(lactide), poly(glycolide), poly(lactide-co-glycolide), poly(L-lactide-co-trimethylene carbonate), poly(dioxanone), poly(butylene succinate), and poly(butylene adipate).

The term “aliphatic polyester” covers—besides polyesters which are made from aliphatic and/or cycloaliphatic components exclusively also polyesters which contain besides aliphatic and/or cycloaliphatic units, aromatic units, as long as the polyester has substantial bio-based content.

In addition to PLA based resins, nonwoven fabrics in accordance with embodiments of the invention may include other polymers derived from an aliphatic component possessing one carboxylic acid group and one hydroxyl group, which are alternatively called polyhydroxyalkanoates (PHA). Examples thereof are polyhydroxybutyrate (PHB), poly-(hydroxybutyrate-co-hydroxyvaleterate) (PHBV), poly-(hydroxybutyrate-co-polyhydroxyhexanoate) (PHBH), polyglycolic acid (PGA), poly-(epsilon-caprolactione) (PCL) and preferably polylactic acid (PLA).

Examples of additional polymers that may be used in embodiments of the invention include polymers derived from a combination of an aliphatic component possessing two carboxylic acid groups with an aliphatic component possessing two hydroxyl groups, and are polyesters derived from aliphatic diols and from aliphatic dicarboxylic acids, such as polybutylene succinate (PBS), polyethylene succinate (PES), polybutylene adipate (PBA), polyethylene adipate (PEA), polytetramethy-lene adipate/terephthalate (PTMAT).

Useful aliphatic polyesters include those derived from semicrystalline polylactic acid. Poly(lactic acid) or polylactide (PLA) has lactic acid as its principle degradation product, which is commonly found in nature, is non-toxic and is widely used in the food, pharmaceutical and medical industries. The polymer may be prepared by ring-opening polymerization of the lactic acid dimer, lactide. Lactic acid is optically active and the dimer appears in four different forms: L,L-lactide, D,D-lactide, D,L-lactide (meso lactide) and a racemic mixture of L,L- and D,D-. By polymerizing these lactides as pure compounds or as blends, poly(lactide) polymers may be obtained having different stereochemistries and different physical properties, including crystallinity. The L,L- or D,D-lactide yields semicrystalline poly(lactide), while the poly(lactide) derived from the D,L-lactide is amorphous.

Generally, polylactic acid based polymers are prepared from dextrose, a source of sugar, derived from field corn. In North America corn is used since it is the most economical source of plant starch for ultimate conversion to sugar. However, it should be recognized that dextrose can be derived from sources other than corn. Sugar is converted to lactic acid or a lactic acid derivative via fermentation through the use of microorganisms. Lactic acid may then be polymerized to form PLA. In addition to corn, other agriculturally-based sugar sources may be used including rice, sugar beets, sugar cane, wheat, cellulosic materials, such as xylose recovered from wood pulping, and the like.

The polylactide preferably has a high enantiomeric ratio to maximize the intrinsic crystallinity of the polymer. The degree of crystallinity of a poly(lactic acid) is based on the regularity of the polymer backbone and the ability to crystallize with other polymer chains. If relatively small amounts of one enantiomer (such as D-) is copolymerized with the opposite enantiomer (such as L-) the polymer chain becomes irregularly shaped, and becomes less crystalline. For these reasons, when crystallinity is favored, it is desirable to have a poly(lactic acid) that is at least 85% of one isomer, at least 90% of one isomer, or at least 95% of one isomer in order to maximize the crystallinity.

In some embodiments, an approximately equimolar blend of D-polylactide and L-polylactide is also useful. In certain embodiments, this blend forms a unique crystal structure having a higher melting point than does either the D-poly(lactide) and L-(polylactide) alone, and has improved thermal stability.

Copolymers, including block and random copolymers, of poly(lactic acid) with other aliphatic polyesters may also be used. Useful co-monomers include glycolide, beta-propiolactone, tetramethylglycolide, beta-butyrolactone, gamma-butyrolactone, pivalolactone, 2-hydroxybutyric acid, alpha-hydroxyisobutyric acid, alpha-hydroxyvaleric acid, alpha-hydroxyisovaleric acid, alpha-hydroxycaproic acid, alpha-hydroxyethylbutyric acid, alpha-hydroxyisocaproic acid, alpha-hydroxy-beta-methylvaleric acid, alpha-hydroxyoctanoic acid, alpha-hydroxydecanoic acid, alpha-hydroxymyristic acid, and alpha-hydroxystearic acid.

Blends of poly(lactic acid) and one or more other aliphatic polyesters, or one or more other polymers may also be used. Examples of useful blends include poly(lactic acid) and poly(vinyl alcohol), polyethylene glycol/polysuccinate, polyethylene oxide, polycaprolactone and polyglycolide.

In certain preferred embodiments, the aliphatic polyester component comprises a PLA based resin. A wide variety of different PLA resins may be used to prepare nonwoven fabrics in accordance with embodiments of the invention. The PLA resin should have proper molecular properties to be spun in spunbond processes. Examples of suitable include PLA resins are supplied from NatureWorks LLC, of Minnetonka, Minn. 55345 such as, grade 6752D, 6100D, and 6202D, which are believed to be produced as generally following the teaching of U.S. Pat. Nos. 5,525,706 and 6,807,973 both to Gruber et al. Other examples of suitable PLA resins may include L130, L175, and LX175, all from Corbion of Arkelsedijk 46, 4206 A C Gorinchem, the Netherlands.

In some embodiments, the inventive nonwoven fabrics may comprise bio-based polymer components of biodegradable products that are derived from an aliphatic component possessing one carboxylic acid group (or a polyester forming derivative thereof, such as an ester group) and one hydroxyl group (or a polyester forming derivative thereof, such as an ether group) or may be derived from a combination of an aliphatic component possessing two carboxylic acid groups (or a polyester forming derivative thereof, such as an ester group) with an aliphatic component possessing two hydroxyl groups (or a polyester forming derivative thereof, such as an ether group).

Additional nonlimiting examples of bio-based polymers include polymers directly produced from organisms, such as polyhydroxyalkanoates (e.g., poly(beta-hydroxyalkanoate), poly(3-hydroxybutyrate-co-3-hydroxyvalerate, NODAX™), and bacterial cellulose; polymers extracted from plants and biomass, such as polysaccharides and derivatives thereof (e.g., gums, cellulose, cellulose esters, chitin, chitosan, starch, chemically modified starch), proteins (e.g., zein, whey, gluten, collagen), lipids, lignins, and natural rubber; and current polymers derived from naturally sourced monomers and derivatives, such as bio-polyethylene, bio-polypropylene, polytrimethylene terephthalate, polylactic acid, NYLON 11, alkyd resins, succinic acid-based polyesters, and bio-polyethylene terephthalate.

In some embodiments, the bio-based polymer may comprise bio-based polyethylene, bio-based polypropylene, and bio-based polyesters, such as bio-based PET, that are derived from a biological source. For example, bio-based polyethylene can be prepared from sugars that are fermented to produce ethanol, which in turn is dehydrated to provide ethylene. An example of a suitable sugar cane derived polyethylene is available from Braskem S.A. under the product name PE SHA7260.

### Optional Components

In some embodiments, the fibers may include one or more additives that are blended with the polymer(s) during the melt extrusion phase. Examples of suitable additives include one or more of colorants, such as pigments (e.g., TiO₂), UV stabilizers, hydrophobic agents, hydrophilic agents, antistatic agent, elastomers, compatibilizers, antioxidants, anti-block agent, slip agents, surfactants, optical brighteners, flame retardants, antimicrobials, such as copper oxide and zinc oxide and the like.

In some embodiments, it may also be useful to optionally treat the nonwoven fabric with finishes containing additives or other chemicals, such as antimicrobial agents, flame retardant agents, catalysts, lubricants, softeners, light stabilizers, antioxidants, colorants such as dyes and/or pigments, antistatic agents, fillers, odor control agents, perfumes and fragrances, and the like, and combinations thereof. Other optional components may be included in the compositions described herein.

### Fabric Properties

According to certain embodiments, for instance, the fabric may comprise a machine direction (MD) tensile strength at max from about 20 N/5 cm to about 75 N/5 cm. In other embodiments, for example, the fabric may comprise a MD tensile strength at max from about 22 N/5 cm to about 65 N/5 cm. In further embodiments, for instance, the fabric may comprise a MD tensile strength at max from about 50 N/5 cm to about 65 N/5 cm. As such, in certain embodiments the fabric may comprise a MD tensile strength at max from at least about any of the following: 20, 25, 26, 27, 28, 29, 30, 50, 60, 70, and 80 N/5 cm, and/or at most about 100, 75, 70, 65, 60, 55, 50, and 45 N/5 cm, (e.g., about 25-100 N/5 cm, about 30-75 N/5 cm, about 45 to 65 N/5 cm, etc.). In general, it should be recognized that MD and CD tensile strengths can vary depending on the basis weight of the fabric. In particular, nonwoven fabrics in accordance with embodiments of the invention may exhibit higher MD and CD tensile strengths than those provided above at basis weights above 30 gsm.

In certain embodiments, for example, the fabric may comprise a cross machine direction (CD) tensile strength at max from about 5 N/5 cm to about 85 N/5 cm. In other embodiments, for instance, the fabric may comprise a CD tensile strength at max from about 6 N/5 cm to about 75 N/5 cm. In some embodiments, for example, the fabric may comprise a CD tensile strength at max from about 7 N/5 cm to about 25 N/5 cm. As such, in certain embodiments, the fabric may comprise a CD tensile strength at max from at least about any of the following: 6, 7, 8, 9, 10, 11, 12, 13, 14, 15, 16, 17, 18, 19, 20, 21, 22, 23, 24, 25, 26, 27, 28, 29, and 30 N/5 cm and/or at most about 85, 80, 75, 70, 65, 60, 55, 50, 45, 40, 35, 30, 29, 28, 27, 26, and 25 N/5 cm (e.g., about 15-85 N/5 cm, about 15-30 N/5 cm, etc.).

In accordance with certain embodiments, for example, the nonwoven fabric may have a basis weight from about 5 grams per square meter (gsm) to about 150 gsm. In other embodiments, for instance, the fabric may have a basis weight from about 8 gsm to about 70 gsm. In certain embodiments, for example, the fabric may comprise a basis weight from about 10 gsm to about 50 gsm. In further embodiments, for instance, the fabric may have a basis weight from about 11 gsm to about 30 gsm. In one embodiment, the fabric may have a basis weight from about 15 gsm to about 25 gsm. As such, in certain embodiments, the fabric may have a basis weight from at least about any of the following: 5, 6, 7, 8, 9, 10, and 11 gsm and/or at most about 150, 100, 70, 60, 50, 40, and 30 gsm (e.g., about 9-60 gsm, about 11-40 gsm, etc.).

According to certain embodiments, for example, the fibers may have a linear mass density from about 0.05 dtex to about 12 dtex. In other embodiments, for instance, the fibers may have a dtex from about 1 dtex to about 10 dtex. In further embodiments, for example, the fibers may have a linear mass density from about 1.2 dtex to about 6 dtex. As such, in certain embodiments, the fibers have a linear mass density from at least about any of the following: 0.6, 0.7, 0.8, 0.9, 1.0, 1, 1.1, 1.2, 1.3, 1.4, 1.5, and 1.6 dtex and/or at most about 2.5, 2.4, 2.3, 2.2, 2.1, 2.0, 1.9, 1.8, and 1.7 dtex (e.g., about 1-2.5 dtex, about 1.1-1.8 dtex, etc.).

For meltblown fibers, the fibers may have a linear mass density from about 0.05 dtex to about 2.0 dtex.

In certain embodiments, bonded nonwoven fabrics in accordance with embodiments of the present invention exhibit improvements in abrasion resistance. Abrasion resistance is often measured with a Rub test where the surface of the fabric is rubbed in a very controlled manner and then loosened fibers are removed and weighed. Fabrics having improved abrasion resistance will exhibit a decrease in the weight of fibers removed in comparison to a less abrasion resistant nonwoven fabric. In some embodiments, nonwoven fabrics exhibited an average weight of material removed during the abrasion testing of less than 5 mg, and in particular, less than 4. mg, as determined in accordance with Test Method NWSP 20.5. Abrasion Test Method NWSP 20.5 is discussed in greater detail below in the Example Section.

In certain embodiments, nonwoven fabrics in accordance with the invention may exhibit a Martindale Abrasion Score of less than 2, and in particular, less than 1.5. In certain embodiments, nonwoven fabrics in accordance with the invention may exhibit a Martindale Abrasion Score from about 1.0 to 2.0, and in particular, from 1.0 to 1.6, and more particularly, from about 1.1 to 1.5. In one particular embodiment, nonwoven fabrics in accordance with the invention may exhibit a Martindale Abrasion Score from about 1.40 to 1.45. The test method for evaluating the Martindale Abrasion Score is discussed in greater detail below in the Example Section.

In certain embodiments, nonwoven fabrics in accordance with the invention may exhibit a Martindale Abrasion Score greater than 1.0, greater than 1.05, greater than 1.10, greater than 1.15, greater than 1.20, greater than 1.25, greater than 1.30, greater than 1.35, greater than 1.40, greater than 1.45, greater than 1.50, greater than 1.55, greater than 1.60, greater than 1.65, greater than 1.70, greater than 1.75, greater than 1.80, greater than 1.85, greater than 1.90, greater than 1.95, and greater than 1.99.

In certain embodiments, nonwoven fabrics in accordance with the invention may exhibit a Martindale Abrasion Score less than 2.0, less than 1.95, less than 1.90, less than 1.85, less than 1.80, less than 1.75, less than 1.70, less than 1.65, less than 1.60, less than 1.55, greater than 1.50, less than 1.45, less than 1.40, less than 1.35, less than 1.30, less than 1.25, less than 1.20, less than 1.15, less than 1.10, less than 1.05, and less than 1.01.

In certain embodiments, bonded nonwoven fabrics in accordance with embodiments of the present invention exhibit improved softness as demonstrated by a nonwoven fabric with a basis weight from 20 to 30 gsm exhibiting a cross direction Handle-o-meter of less than 7.0 grams (g), such as less than 7.9 grams or less than 7.5 grams. Handle-O-Meter was measured in accordance with NWSP 90.3, which is discussed in greater detail below in the Example Section.

In certain embodiments, bonded nonwoven fabrics in accordance with embodiments of the present invention exhibit improved softness as demonstrated by nonwoven fabric with a basis weight of 20 to 30 gsm exhibiting a machine direction handle-o-meter of less than 3.9 grams (g), such as less than 3.8 grams or less than 3.78 grams.

In certain embodiments, nonwoven fabrics in accordance with embodiments of the invention exhibit improvements in one or more of tensile strengths, elongations, abrasion resistance, and softness in comparison to a similarly prepared nonwoven fabric with the exception that the similarly fabric was point bonded with a bonding pattern having a bond point packing of less than 3.5 mm⁻¹, such as less than 4.0 mm⁻¹. In certain embodiments, the similarly prepared nonwoven fabric was point bonded with a bonding pattern in which the percent surface bonding area of the nonwoven fabric 18.1%. That is, the overall bonding point pattern is the same in the similarly prepared nonwoven fabric, with the exception that the surface area of the individual bond points is larger such that the overall percent bonded surface area of the nonwoven fabric is greater, which also results in a decrease in the bond point packing. In certain embodiments, the similarly prepared fabric is substantially identical (for example polymer chemistry, fiber structure, and extrusion conditions) to the inventive fabric with the exception of the foregoing bonding pattern in the comparison fabric having a bond point packing of less than 3.5 mm⁻¹, such as less than 4.0 mm⁻¹. Some variations in process conditions used in the similarly prepared nonwoven fabric may exist, such as, for example, slight variations in calender temperatures and pressures.

In certain embodiments, nonwoven fabrics in accordance with embodiments of the present invention may exhibit tensile strengths that are 10% greater in comparison to a similarly prepared nonwoven fabric having a bond point packing of less than 3.5 mm⁻¹, such as less than 4.0 mm⁻¹. In some embodiments, the nonwoven fabric may exhibit a tensile strength that is from 10% to 50%, such as 12 to 30%, 12 to 25%., 12 to 24%, or 12 to 20% greater than the tensile strength of a similarly prepared nonwoven fabric having a bond point packing of less than 3.5 mm⁻¹, such as less than 4.0 mm⁻¹.

In certain embodiments, nonwoven fabrics in accordance with embodiments of the present invention may exhibit tensile strengths that are 10% greater in comparison to a similarly prepared nonwoven fabric having a bonded surface area greater than 12%, such as 18.1%. In some embodiments, the nonwoven fabric may exhibit a tensile strength that is from 10% to 30%, such as 12 to 20%, greater than the tensile strength of a similarly prepared nonwoven fabric having a bonded surface area greater than 12%, such as 18.1%.

In particular, nonwoven fabrics in accordance with the present invention may exhibit increases in machine direction (MD) tensile strengths that are from about 10 to 50% greater in comparison to a similarly prepared nonwoven fabric having a bond point packing of less than 3.5 mm⁻¹, such as less than 4.0 mm⁻¹. In some embodiments, the inventive nonwoven fabrics may exhibit an increase in MD tensile strength ranging from about 10 to 30%, such as from about 12 to 20%, or from about 12 to 15% in comparison to a similarly prepared nonwoven fabric having a bond point packing of less than 3.5 mm⁻¹, such as less than 4.0 mm⁻¹.

In addition, nonwoven fabrics in accordance with the present invention may exhibit increases in cross direction (CD) tensile strengths that are from about 10 to 50% greater in comparison to a similarly prepared nonwoven fabric having a bond point packing of less than 3.5 mm⁻¹, such as less than 4.0 mm⁻¹. In some embodiments, the inventive nonwoven fabrics may exhibit an increase in CD tensile strength ranging from about 10 to 30%, such as from about 15 to 25%, from about 18 to 24%, or from about 19 to 21% in comparison to a similarly prepared nonwoven fabric having a bond point packing of less than 3.5 mm⁻¹, such as less than 4.0 mm⁻¹.

In certain embodiments, nonwoven fabrics in accordance with the present invention may exhibit increases in machine direction (MD) tensile strengths that are from about 10 to 50% greater in comparison to a similarly prepared nonwoven fabric having a bonded surface area greater than 12%, such as 18.1%. In some embodiments, the inventive nonwoven fabrics may exhibit an increase in MD tensile strength ranging from about 10 to 30%, such as from about 12 to 20%, or from about 12 to 15% in comparison to a similarly prepared nonwoven fabric having a bonded surface area greater than 12%, such as 18.1%.

In addition, nonwoven fabrics in accordance with the present invention may exhibit increases in cross direction (CD) tensile strengths that are from about 10 to 50% greater in comparison to a similarly prepared nonwoven fabric having a bonded surface area greater than 12%, such as 18.1%. In some embodiments, the inventive nonwoven fabrics may exhibit an increase in CD tensile strength ranging from about 10 to 30%, such as from about 15 to 25%, from about 18 to 24%, or from about 19 to 21% in comparison to a similarly prepared nonwoven fabric having a bonded surface area greater than 12%, such as 18.1%.

In some embodiments, nonwoven fabrics in accordance with the present invention may exhibit increases in percent elongation that are from about 4 to 50% in comparison to a similarly prepared nonwoven fabric having a bond point packing of less than 3.5 mm⁻¹, such as less than 4.0 mm⁻¹. In some embodiments, the inventive nonwoven fabrics may exhibit an increase in percent elongation ranging from about 4 to 25%, such as from about 5 to 20%, or from about 5 to 15%, in comparison to a similarly prepared nonwoven fabric having a bond point packing of less than 3.5 mm⁻¹, such as less than 4.0 mm⁻¹.

In some embodiments, nonwoven fabrics in accordance with the present invention may exhibit increases in percent elongation that are from about 4 to 50% in comparison to a similarly prepared nonwoven fabric having a bonded surface area greater than 12%, such as 18.1%. In some embodiments, the inventive nonwoven fabrics may exhibit an increase in percent elongation ranging from about 4 to 25%, such as from about 5 to 20%, or from about 5 to 15%, in comparison to a similarly prepared nonwoven fabric having a bonded surface area greater than 12%, such as 18.1%.

In certain embodiments nonwoven fabrics in accordance with the present invention may exhibit an improvement in abrasion resistance as exemplified by a percent difference in the Martindale Abrasion Score of 10 to 30% in comparison to a similarly prepared nonwoven fabric having a bond point packing of less than 3.5 mm⁻¹, such as less than 4.0 mm⁻¹. In some embodiments, the inventive nonwoven fabrics may an improvement in abrasion resistance as exemplified by a percent difference in the Martindale Abrasion Score of from about 10 to 25%, such as from about 12 to 24%, or from about 18 to 22%, in comparison to a similarly prepared nonwoven fabric having a bond point packing of less than 3.5 mm⁻¹, such as less than 4.0 mm⁻¹.

In certain embodiments nonwoven fabrics in accordance with the present invention may exhibit an improvement in abrasion resistance as exemplified by a percent difference in the Martindale Abrasion Score of 10 to 30% in comparison to a similarly prepared nonwoven fabric having a bonded surface area greater than 12%, such as 18.1%. In some embodiments, the inventive nonwoven fabrics may an improvement in abrasion resistance as exemplified by a percent difference in the Martindale Abrasion Score of from about 10 to 25%, such as from about 12 to 24%, or from about 18 to 22%, in comparison to a similarly prepared nonwoven fabric having a bonded surface area greater than 12%, such as 18.1%.

In some embodiments, nonwoven fabrics in accordance with embodiments of the invention exhibited an average percent decrease in the weight of material removed during the abrasion testing (in accordance with Test Method NWSP 20.5) from 5 to 150% in comparison to a similarly prepared nonwoven fabric having a bond point packing of less than 3.5 mm⁻¹, such as less than 4.0 mm⁻¹. In certain embodiments, the inventive nonwoven fabrics may exhibit an average percent decrease in the weight of material removed during the abrasion testing from 8 to 120%, such as 9 to 95% in comparison to a similarly prepared nonwoven fabric having a bond point packing of less than 3.5 mm⁻¹, such as less than 4.0 mm⁻¹.

In some embodiments, nonwoven fabrics in accordance with embodiments of the invention exhibited an average percent decrease in the weight of material removed during the abrasion testing (in accordance with Test Method NWSP 20.5) from 5 to 150% in comparison to a similarly prepared nonwoven fabric having a bonded surface area greater than 12%, such as 18.1%. In certain embodiments, the inventive nonwoven fabrics may exhibit an average percent decrease in the weight of material removed during the abrasion testing from 8 to 120%, such as 9 to 95% in comparison to a similarly prepared nonwoven fabric having a bonded surface area greater than 12%, such as 18.1%.

In some embodiments, nonwoven fabrics in accordance with embodiments of the invention exhibited improvements in softness as demonstrated by an average improvement of Handle-O-Meter values from about 5 to 20% in comparison to a similarly prepared nonwoven fabric having a bond point packing of less than 3.5 mm⁻¹, such as less than 4.0 mm⁻¹. In certain embodiments, the inventive nonwoven fabrics may exhibit an average improvement of Handle-O-Meter values from about 6 to 15%., such as 8 to 10% in comparison to a similarly prepared nonwoven fabric having a bond point packing of less than 3.5 mm⁻¹, such as less than 4.0 mm⁻¹.

In some embodiments, nonwoven fabrics in accordance with embodiments of the invention exhibited improvements in softness as demonstrated by an average improvement of Handle-O-Meter values from about 5 to 20% in comparison to a similarly prepared nonwoven fabric having a bonded surface area greater than 12%, such as 18.1%. In certain embodiments, the inventive nonwoven fabrics may exhibit an average improvement of Handle-O-Meter values from about 6 to 15%, such as 8 to 10% in comparison to a similarly prepared nonwoven fabric having a bonded surface area greater than 12%, such as 18.1%.

In certain embodiments, nonwoven fabrics in accordance with embodiments of the invention exhibited improvements in softness (measured with Handle-O-Meter) from about 5 to 20% in comparison to a similarly prepared nonwoven fabric having a bond point packing of less than 3.5 mm⁻¹, such as less than 4.0 mm⁻¹. In certain embodiments, the inventive nonwoven fabrics may exhibit an average improvement is softness from about 6 to 15%, such as 8 to 10% in comparison to a similarly prepared nonwoven fabric having a bond point packing of less than 3.5 mm⁻¹, such as less than 4.0 mm⁻¹.

In certain embodiments, nonwoven fabrics in accordance with embodiments of the invention exhibited improvements in softness (measured with Handle-O-Meter) from about 5 to 20% in comparison to a similarly prepared nonwoven fabric having a bonded surface area greater than 12%, such as 18.1%. In certain embodiments, the inventive nonwoven fabrics may exhibit an average improvement is softness from about 6 to 15%, such as 8 to 10% in comparison to a similarly prepared nonwoven fabric having a bonded surface area greater than 12%, such as 18.1%.

In certain embodiments, nonwoven fabrics in accordance with embodiments of the invention may be characterized by having a percent bonded area from about 9.6 to 10.4%, an average individual bond point surface area from about 0.15 to 2.0 mm², an average bond point packing value from about 6.75 to 7.25 mm⁻¹, a bond point density from about 50 to 60 individual bond points per square centimeter, a Martindale Abrasion Score from about 1.0 to 1.5 (e.g., 1.4 to 1.5), a cross direction Handle-o-Meter from about 6.6 to 7.2 grams (for a nonwoven fabric having a basis weight from 20 to 30 gsm), a machine direction Handle-O-Meter from about 3.5 to 3.95 grams, and average abrasion resistance as determined by the weight of removed material from 3.2 to 5.5 grams.

In certain embodiments, nonwoven fabrics in accordance with embodiments of the invention may be characterized by having a percent bonded area from about 9.8 to 10%, an average individual bond point surface area from about 0.16 to 0.19 mm², an average bond point packing value from about 7.0 to 7.3 mm⁻¹, a bond point density from about 52 to 58 individual bond points per square centimeter, a Martindale Abrasion Score from about 1.42 to 1.45, a cross direction Handle-o-Meter from about 6.7 to 7.0 grams (for a nonwoven fabric having a basis weight from 20 to 30 gsm), a machine direction Handle-O-Meter from about 3.6 to 3.9 grams, and average abrasion resistance as determined by the weight of removed material from 3.4 to 3.6 grams.

In certain embodiments, nonwoven fabrics in accordance with embodiments of the invention may be characterized by having a percent bonded area from about 9.6 to 10.2%, an average individual bond surface area from about 0.15 to 0.25 mm², an average bond point packing value from about 6.75 to 7.25 mm⁻¹, and an average increase in tensile strengths that are 10% greater, such as from about 10 to 50%, from about 12 to 24%, or from about 12 to 22%, in comparison to a similarly prepared nonwoven fabric having a bond point packing of less than 3.5 mm⁻¹, such as less than 4.0 mm⁻¹.

In certain embodiments, nonwoven fabrics in accordance with embodiments of the invention may be characterized by having a percent bonded area from about 9.6 to 10.2%, an average individual bond surface area from about 0.15 to 0.25 mm², an average bond point packing value from about 6.75 to 7.25 mm⁻¹, and an average increase in tensile strengths that are 10% greater, such as from about 10 to 50%, from about 12 to 24%, or from about 12 to 22%, in comparison to a similarly prepared nonwoven fabric having a bonded surface area greater than 12%, such as 18.1%.

In certain embodiments, nonwoven fabrics in accordance with embodiments of the invention may be characterized by having a percent bonded area from about 9.6 to 10.2%, an average individual bond surface area from about 0.15 to 0.25 mm², an average bond point packing value from about 6.75 to 7.25 mm⁻¹, and an average increase in percent elongation that is from about 4 to 50%, such as from about 4 to 20%, from about 4 to 15%, or from about 4 to 14%, in comparison to a similarly prepared nonwoven fabric having a bond point packing of less than 3.5 mm⁻¹, such as less than 4.0 mm⁻¹.

In certain embodiments, nonwoven fabrics in accordance with embodiments of the invention may be characterized by having a percent bonded area from about 9.6 to 10.2%, an average individual bond surface area from about 0.15 to 0.25 mm², an average bond point packing value from about 6.75 to 7.25 mm⁻¹, and an average increase in percent elongation that is from about 4 to 50%, such as from about 4 to 20%, from about 4 to 15%, or from about 4 to 14%, in comparison to a similarly prepared nonwoven fabric having a bonded surface area greater than 12%, such as 18.1%.

In certain embodiments, nonwoven fabrics in accordance with embodiments of the invention may be characterized by having a percent bonded area from about 9.6 to 10.2%, an average individual bond surface area from about 0.15 to 0.25 mm², an average bond point packing value from about 6.75 to 7.25 mm⁻¹, and one or more of the following properties:
i) an improvement in abrasion resistance as exemplified by a percent difference in the Martindale Abrasion Score of 10 to 30% in comparison to a similarly prepared nonwoven fabric having a bond point packing of less than 1 mm⁻¹;
ii) an average percent decrease in the weight of material removed during the abrasion testing (in accordance with Test Method NWSP 20.5) from 5 to 150%, such as from about 8 to 120% or 9 to 95% in comparison to a similarly prepared nonwoven fabric having a bond point packing of less than 1 mm⁻¹; and
iii) improvements in softness as demonstrated by an average improvement of Handle-O-Meter values from about 5 to 20%, such as from about 6 to 15% or 8 to 10%, in comparison to a similarly prepared nonwoven fabric having a bond point packing of less than 1 mm⁻¹.

In certain embodiments, nonwoven fabrics in accordance with embodiments of the invention may be characterized by having a percent bonded area from about 9.6 to 10.2%, an average individual bond surface area from about 0.15 to 0.25 mm², an average bond point packing value from about 6.75 to 7.25 mm⁻¹, and one or more of the following properties:
i) an improvement in abrasion resistance as exemplified by a percent difference in the Martindale Abrasion Score of 10 to 30% in comparison to a similarly prepared nonwoven fabric having a bonded surface area greater than 12%, such as 18.1%;
ii) an average percent decrease in the weight of material removed during the abrasion testing (in accordance with Test Method NWSP 20.5) from 5 to 150%, such as from about 8 to 120% or 9 to 95% in comparison to a similarly prepared nonwoven fabric having a bonded surface area greater than 12%, such as 18.1%; and
iii) improvements in softness as demonstrated by an average improvement of Handle-O-Meter values from about 5 to 20%, such as from about 6 to 15% or 8 to 10%, in comparison to a similarly prepared nonwoven fabric having a bonded surface area greater than 12%, such as 18.1%.

In certain embodiments, nonwoven fabrics in accordance with embodiments of the invention may be characterized by having a percent bonded area from about 9.6 to 10.2%, an average individual bond surface area from about 0.10 to 0.25 mm², an average bond point packing value from about 6.75 to 7.25 mm⁻¹, and one or more of the following properties:
i) an improvement in abrasion resistance as exemplified by a percent difference in the Martindale Abrasion Score of 10 to 30% in comparison to a similarly prepared nonwoven fabric having a bond point packing of less than 1 mm⁻¹;
ii) an average percent decrease in the weight of material removed during the abrasion testing (in accordance with Test Method NWSP 20.5) from 5 to 150%, such as from about 8 to 120% or 9 to 95% in comparison to a similarly prepared nonwoven fabric having a bond point packing of less than 3.5 mm⁻¹, such as less than 4.0 mm⁻¹;
iii) improvements in softness as demonstrated by an average improvement of Handle-O-Meter values from about 5 to 20%, such as from about 6 to 15% or 8 to 10%, in comparison to a similarly prepared nonwoven fabric having a bond point packing of less than 3.5 mm⁻¹, such as less than 4.0 mm⁻¹;
iv) an increase in machine direction (MD) tensile strengths that are from about 10 to 50% greater, such as from about 10 to 30%, such as from about 12 to 20%, or from about 12 to 15% greater, in comparison to a similarly prepared nonwoven fabric having a bond point packing of less than 3.5 mm⁻¹, such as less than 4.0 mm⁻¹;
v) an increase in cross direction (CD) tensile strengths that are from about 10 to 50% greater, such as from about 10 to 30%, such as from about 15 to 25%, from about 18 to 24%, or from about 19 to 21%, in comparison to a similarly prepared nonwoven fabric having a bond point packing of less than 3.5 mm⁻¹, such as less than 4.0 mm⁻¹; and
vi) an increase in percent elongation that is from about 4 to 50%, such as from about 5 to 20%, or from about 5 to 15%, in comparison to a similarly prepared nonwoven fabric having a bond point packing of less than 3.5 mm⁻¹, such as less than 4.0 mm-.

In certain embodiments, nonwoven fabrics in accordance with embodiments of the invention may be characterized by having a percent bonded area from about 9.6 to 10.2%, an average individual bond surface area from about 0.15 to 0.25 mm², an average bond point packing value from about 6.75 to 7.25 mm⁻¹, and one or more of the following properties: i) an improvement in abrasion resistance as exemplified by a percent difference in the Martindale Abrasion Score of 10 to 30% in comparison to a similarly prepared nonwoven fabric having a bonded surface area greater than 12%, such as 18.1%;
ii) an average percent decrease in the weight of material removed during the abrasion testing (in accordance with Test Method NWSP 20.5) from 5 to 150%, such as from about 8 to 120% or 9 to 95% in comparison to a similarly prepared nonwoven fabric having a bonded surface area greater than 12%, such as 18.1%;
iii) an improvement in softness as demonstrated by an average improvement of Handle-O-Meter values from about 5 to 20%, such as from about 6 to 15% or 8 to 10%, in comparison to a similarly prepared nonwoven fabric having a bonded surface area greater than 12%, such as 18.1%;
iv) an increase in machine direction (MD) tensile strengths that are from about 10 to 50% greater, such as from about 10 to 30%, such as from about 12 to 20%, or from about 12 to 15% greater, in comparison to a similarly prepared nonwoven fabric having a bonded surface area greater than 12%, such as 18.1%;
v) an increase in cross direction (CD) tensile strengths that are from about 10 to 50% greater, such as from about 10 to 30%, such as from about 15 to 25%, from about 18 to 24%, or from about 19 to 21%, in comparison to a similarly prepared nonwoven fabric having a bonded surface area greater than 12%, such as 18.1%;
and
vi) an increase in percent elongation that is from about 4 to 50%, such as from about 5 to 20%, or from about 5 to 15%, in comparison to a similarly prepared nonwoven fabric having a bonded surface area greater than 12%, such as 18.1%.

### System and Method for Preparing the Nonwoven Fabric

Certain aspects of the invention provide systems and methods for preparing a bonded nonwoven fabric in accordance with embodiments described previously discussed.

With reference to FIG. 8, for example, a schematic diagram of a spunbond nonwoven fabric preparation system in accordance with certain embodiments of the invention is illustrated and broadly designated by reference character 100a. As shown in FIG. 8, a first polymer source (i.e. hopper) 102 is in fluid communication with the spin beam 104 via the extruder 106.

In certain embodiments, the first polymer source may provide a stream of a molten or semi-molten polymer resin. Following extrusion, the extruded polymer stream are introduced into the spin beam 104 at which point the streams enter a plurality of spinnerets (not shown) for spinning into filaments. Following spinning, the spun filaments may then be drawn (i.e. attenuated) via a drawing unit (not shown) and randomized in a diffuser. The spin beam 104 produces a curtain of filaments 108 that is deposited on the collection surface 110 to produce a web of filaments. At this stage, the filaments may comprise a web 112 of filaments that are unbonded or slightly bonded to each other.

A calender bonding unit 116 comprising a calender is disposed downstream of the collection surface 110 and is configured and arranged to thermally point adjacent filaments to each other and also impart a bond pattern onto the surface in accordance with embodiments of the invention as discussed previously. As discussed in greater detail below, the calender comprises a pair of cooperating rolls in which a first roll 116a comprises an engraved patterned roll having a plurality of bonding points extending from a surface thereof, and the second roll 116b comprises smooth or anvil surface. During bonding the web 112 of filaments passes between the pair cooperating rolls, which are heated to a temperature that is sufficient to soften at least one polymer component comprising filaments of web 112 to produce a bonded nonwoven fabric 124. In certain embodiments, the bonded nonwoven fabric 124 moves to a winder 118, where the fabric is then wound onto rolls.

In some embodiments, an optional pair of cooperating rolls 120 (also referred to herein as a “press roll”) stabilize the web of filaments by compressing the web before delivery to the calender 116 for bonding. In some embodiments, for example, the press roll may include a ceramic coating deposited on a surface thereof. In certain embodiments, for instance, one roll of the pair of cooperating rolls 120 may be positioned above the collection surface 110, and a second roll of the pair of cooperating rolls 120 may be positioned below the collection surface 110. In some embodiments, the system may also include a hot air knife (not shown) that exposes the web 112 to a stream of heated gas, such as air, to lightly bond and stabilize the web.

In some embodiments, the system 100a may further comprise a vacuum source 128 disposed below collection surface 110. Vacuum source 128 provides a vacuum that helps draw and pull the curtain of filaments 108 onto collection surface 110.

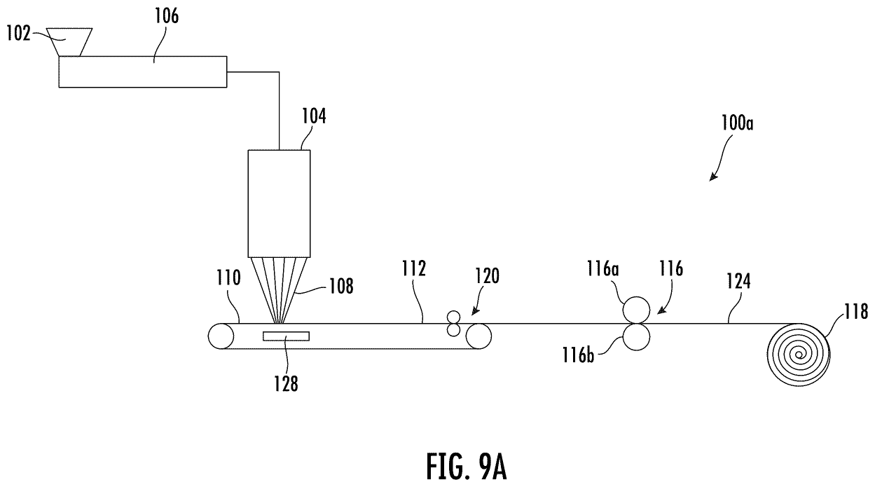

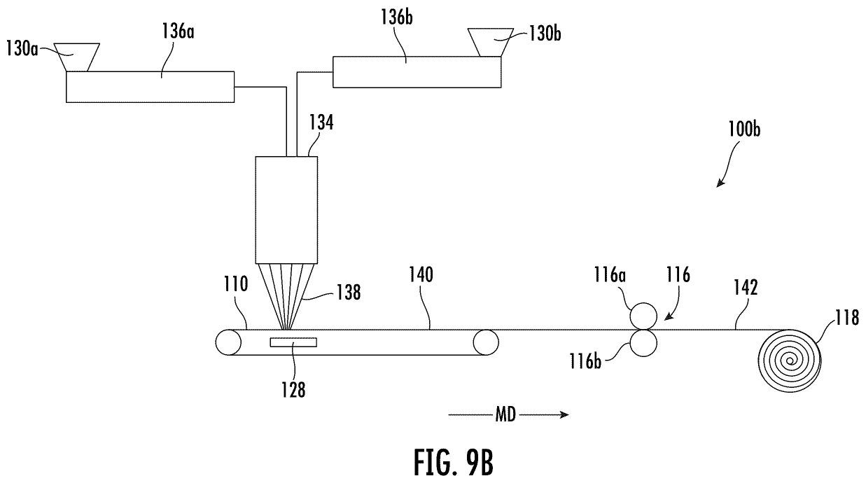

With reference to FIG. 9, a further aspect of a system and method of preparing a nonwoven fabric in accordance with at least one embodiment of the invention is illustrated and broadly designated by reference character 100b. In this embodiment, system 100b may be configured and arranged to produce multicomponent filaments, such as bicomponent filaments.

System 100b includes a first polymer source (i.e. hopper) 130a that is in fluid communication with the spin beam 134 via the extruder 136a. A second polymer source (i.e. hopper) 130b is also in fluid communication with the spin beam 134 via extruder 136b. In the preparation of multicomponent fabrics, first polymer source may provide a stream of a first polymer resin, and the second polymer source may provide a stream of a second polymer resin. In melt spinning applications, the polymer streams are typically in a molten or semi-molten state. The first polymer resin and the second polymer resin may be different polymers, or may be the same polymers depending on the desired application and desired properties of the nonwoven fabric.

Following extrusion, the extruded polymer streams are introduced into the spin beam 134 at which point the streams enter a plurality of spinnerets (not shown) for spinning into filaments. Following spinning, the spun filaments may then be drawn (i.e. attenuated) via a drawing unit (not shown) and randomized in a diffuser. The spin beam 134 produces a curtain of multicomponent filaments 138 that is deposited on the collection surface 110 to produce a web 140 of filaments. At this stage, the filaments may comprise a web 140 of multicomponents filaments that are unbonded or slightly bonded to each other.

A calender bonding unit 116 comprising a calender is disposed downstream of the collection surface 110 and is configured and arranged to thermally point adjacent filaments to each other and also impart a bond pattern onto the surface in accordance with embodiments of the invention as discussed previously. As discussed in greater detail below, the calender comprises a pair of cooperating rolls in which a first roll 116a comprises an engraved patterned roll having a plurality of bonding points extending from a surface thereof, and the second roll 116b comprises smooth or anvil surface. During bonding, the web 140 of filaments passes between the pair cooperating rolls, which are heated to a temperature that is sufficient to soften at least one polymer component comprising filaments of web 140 such that the softened polymer component fuses and bonds to adjacent filaments within the web to produce a bonded nonwoven fabric 142. In certain embodiments, the bonded nonwoven fabric 142 moves to a winder 118, where the fabric is then wound onto rolls.

As in the previously discussed embodiment, system 100b may also include an optional pair of cooperating rolls 120, optional hot air knife, and vacuum source 128.

Embodiments of the invention, may also include multilayered nonwoven fabrics having 2 to 10 layers, such as 2 to 5, and in particular, 2 to 3 layers.

Various layers of the nonwoven fabric may include one or more spunbond layers, one or more carded layers, one or more air laid layers, one or more meltblown layers, and the like.

In certain embodiments, the bonded nonwoven fabric may include a layer comprising monocomponent filaments and a second layer comprising multicomponent filaments, such as bicomponent filaments.

In embodiments in which the bonded nonwoven fabric includes multiple layers, the system may include additional fiber forming devices as desired. For example, systems in accordance with embodiments of the invention may include one or more meltblown beams, one or more devices for preparing carded fabric layers, one or more devices for preparing airlaid fabric layers, and the like. Such additional devices may be the same manufacturing line with the other fiber forming devices to provide a continuous system. Alternatively, one or more additional layers may be provided from a supply roll onto which a previously prepared nonwoven fabric was wound.

In certain embodiments, the bonded nonwoven fabric may include at least one spunbond layer comprising filaments having no crimping or low crimping, and a least one layer comprising crimped filaments.

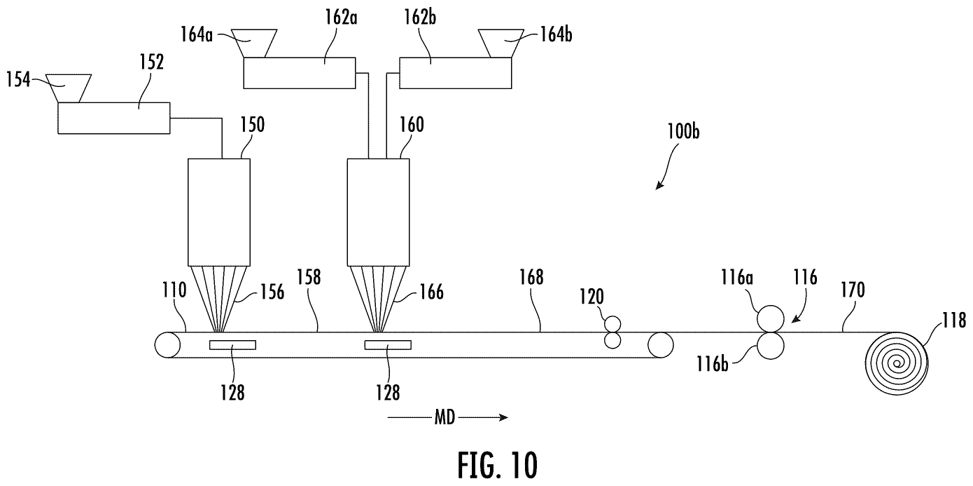

With reference to FIG. 10, a further aspect of a system and method of preparing a nonwoven fabric in accordance with at least one embodiment of the invention is illustrated and broadly designated by reference character 100c. In system 100c, the system includes at least two spin beams and is configured and arranged to produce a bonded nonwoven fabric having at least two layers.

As shown, system 100c includes a first spinbeam 150 in fluid communication with extruder 152 and first polymer source (hopper 154). Following extrusion, the extruded polymer stream is introduced into the spin beam 150 at which point the stream enters a plurality of spinnerets (not shown) for spinning into filaments. Following spinning, the spun filaments may then be drawn (i.e. attenuated) via a drawing unit (not shown) and randomized in a diffuser. The spin beam 150 produces a curtain of filaments 156 that is deposited on the collection surface 110 to produce a web of filaments. At this stage, the filaments may comprise a web 158 of filaments that are unbonded or slightly bonded to each other.

A second spinbeam 160 is disposed downstream of the first spinbeam 150. The second spin beam is in fluid communication with second extruder 162a (which is in communication with second polymer source (hopper 164a)) and third extruder 162b (which is in communication with third polymer source (hopper 164b)).

Following extrusion, the extruded polymer streams from extruders 162a, 162b are introduced into the spin beam 160 at which point the streams enter a plurality of spinnerets (not shown) for spinning into multicomponent filaments. Following spinning, the spun filaments may then be drawn (i.e. attenuated) via a drawing unit (not shown) and randomized in a diffuser. The spin beam 160 produces a curtain of multicomponent filaments 166 that is deposited overlying web 158 produce a composite web 168 comprising at least two layers. At this stage, the filaments of web 158 may be unbonded or slightly bonded to each other.

As in the previously discussed embodiments, a calender bonding unit 116 comprising a calender is disposed downstream of the collection surface 110 and is configured and arranged to thermally point adjacent filaments to each other and also impart a bond pattern onto the surface in accordance with embodiments of the invention as discussed previously. During bonding, the web 168 of filaments passes between the pair cooperating rolls, which are heated to a temperature that is sufficient to soften at least one polymer component comprising filaments of web 168 to produce a bonded nonwoven fabric 170. In certain embodiments, the bonded nonwoven fabric 170 moves to a winder 118, where the fabric is then wound onto rolls.

As in the previously discussed embodiment, system 100c may also include an optional pair of cooperating rolls 120 and vacuum source 128.

In certain embodiments, the nonwoven fabric may be air through bonded prior to thermal point bonding via the calender bonding unit.

In accordance with certain embodiments, for instance, bonding the web to form the bonded nonwoven fabric comprises thermal point bonding the web with heat and pressure via a calender having a pair of cooperating rolls including a patterned roll. The patterned roll imparts a three-dimensional geometric bonding pattern onto the nonwoven fabric.

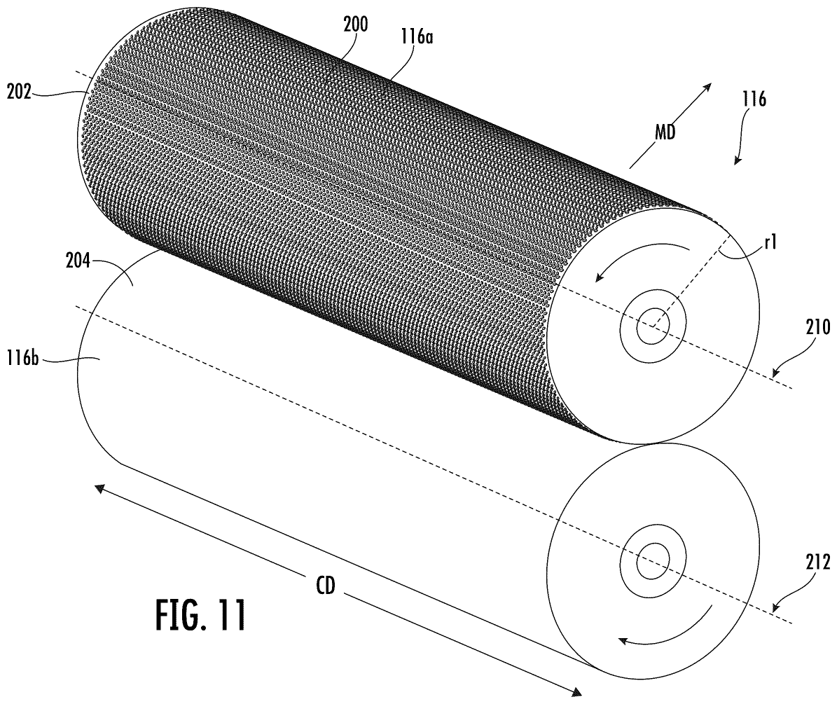

FIG. 11 illustrates a calender bonding unit 116 that is in accordance with at least one embodiment of the present disclosure. In certain embodiments, the calender bonding unit comprises a pair of cylindrically shaped rolls 116a. 116b that arc positioned in a cooperating opposing relationship for receiving a nonwoven fabric therebetween. In the illustrated embodiment, the patterned roll 116a, includes a plurality of individual raised bonding points 200 that extend radially outward from a surface 202 of patterned roll 116a, and are arranged to define one or more patterns on the surface 202 of patterned roll 116a.

Roll 116b (also referred to as an anvil roll) has a generally smooth surface 204. However, in some embodiments, both rolls 116a. 116b may be patterned to include a plurality of individual raised bonding points 200 that extend radially outwardly from surfaces 202, 204.

Rolls 116a, 116b both include a central axis 210, 212, respectively, that extends laterally in the C) direction. Central axis 210, 212 of rolls 116a, 116b are substantially perpendicular with the MD direction of the system for making the inventive nonwoven fabric (see FIGS. 8-10, systems 106a-106c). Rolls 116a, 116b also each include radial axis r1, r2 that extend radially outward from central axis 210, 212.

The bonding points collectively are arranged and distributed across the surface of the pattern roll to define a first pattern that is imparted to the nonwoven sheet when subjected to thermal bonding by being passed between rolls 116a, 116b of calender bonding unit 116.

In certain embodiments, the patterned roll 116a comprises a plurality of arrays in which each array comprises a plurality of spaced apart individual bonding points that extend across the surface of the patterned roll 116a. The arrays of bonding points on the surface of the patterned roll 116a are configured and arranged to impart a plurality of arrays of thermal bond points in the machine direction, cross direction and diagonal direction of a nonwoven fabric subjected to thermal point bonding via calender bonding unit 116.

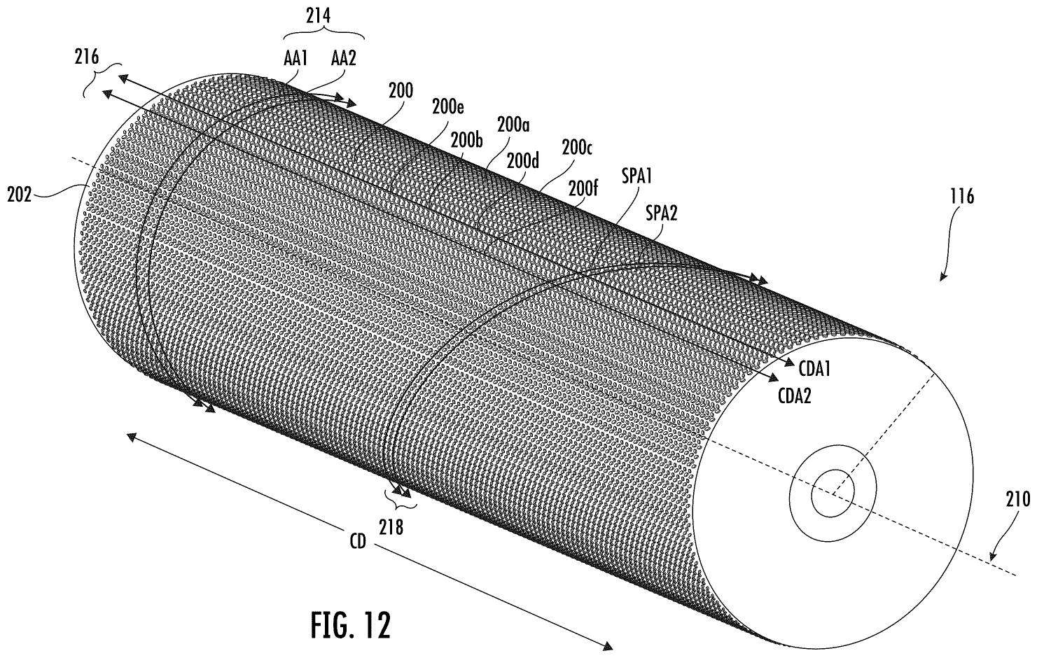

Turning now to FIG. 12, to impart a plurality of arrays of bond points that extend in the machine direction of the nonwoven fabric, the patterned roll 116a includes a plurality of pairs of annular arrays AA1, AA2, in which each annular array AA1, AA2 of the pair 214 comprises a plurality of spaced apart bonding points 200 that are disposed radially about the circumference of patterned roll 116a. Typically, the individual bonding points of each annular array are disposed in a same plane defined by a radial axis r1. The annular arrays comprise a plurality of array pairs 214 in which each array pair comprises a first annular array AA1 defining a first member of the array annular array pair 214 and a second annular array AA2 defining a second member of the annular array pair 214. The plurality of annular array pairs 214 define a pattern in which first annular array AA1 and second annular array AA2 alternate in a repeating pattern across the surface of patterned roll 116a in the cross direction of the roll.

In certain embodiments, the individual bonding points 200 of second annular array AA2 are radially offset relative to adjacent bonding points of the first annular arrays AA1. In other words, adjacent bond points of the first and second annular arrays are not aligned with each other on the surface of the patterned roll in the cross direction of the roll, while the bonding points of every other annular array are substantially aligned in the cross direction of the roll. For example, in FIG. 12 bonding points 200a and 200c which are both in separate annular arrays AA2 are aligned with each other in the cross direction while bonding points 200b and 200d, which are in the first annular arrays AA1 are not aligned with bonding points 200a and 200c in the cross direction of the patterned roll 116a.

In certain embodiments, the patterned roll 116a also includes arrays of individual spaced apart bonding points 200 that extend in the cross direction of the patterned roll 116a. In addition, the arrays extending in the cross direction of the patterned roll 116a comprise a plurality of cross direction array pairs 216 in which each array pair comprises a first cross direction array CDA1 defining a first member of the cross direction array pair 216 and a second cross direction array CDA2 defining a second member of the array pair. The plurality of cross direction array pairs 216 define a pattern in which first cross direction array CDA1 and second cross direction array CDA2 alternate radially about the circumference of the patterned roll in a repeating pattern. In certain embodiments, first cross direction array CDA1 and second cross direction array CDA2 extend across the surface 202 of the roll in a direction that substantially parallel to the central axis 210 of the roll.

In certain embodiments, the individual bonding points 200 of the first cross direction array CDA1 are offset in the cross direction relative to adjacent bonding points of the second cross direction array CDA2. In other words, adjacent bond points of the first and second cross direction arrays are not radially aligned with each other on the surface of the patterned roll, while the bonding points of every other cross direction array are substantially radially aligned across the surface of the patterned roll. For example, in FIG. 12 bonding points 200a and 200e which are both in separate cross direction arrays CDA2 are radially aligned with each other while bonding points 200b and 200f, which are in the first cross direction arrays CDA1 are not radially aligned with bonding points 200a and 200e of the patterned roll 116a.

In certain embodiments, the patterned roll 116a also includes a plurality of spiral-like arrays comprising a plurality of spaced apart bonding points that are configured and arranged in a spiral like pattern that circumferentially extends about the outer surface of the pattern roll 116a. In this regard, FIG. 12 illustrates pairs of spiral arrays 218 having a first spiral array SPA1 and a second spiral array SPA2 in which the plurality of pairs of spiral arrays are spaced apart from each other across the surface of the patterned roll 116a. The spiral like arrays impart a pattern onto the nonwoven sheet comprising a plurality of arrays that extend in a diagonal direction across the surface of the bonded nonwoven fabric (see, for example, FIG. 1, arrays A5, A6).

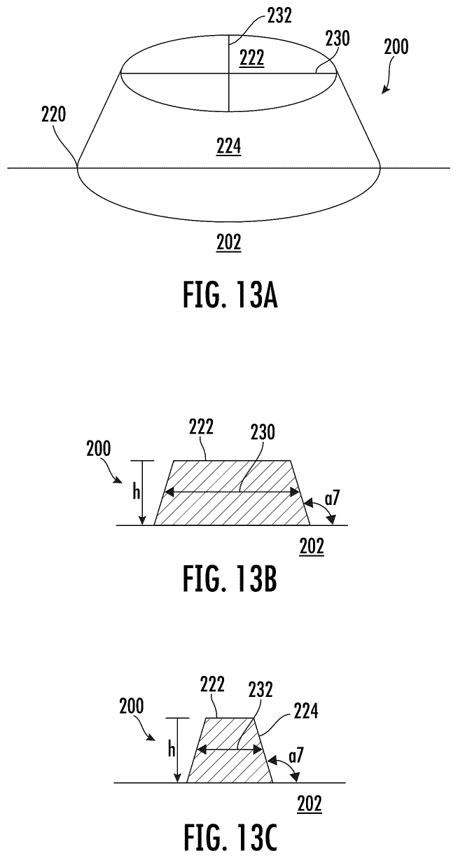

With reference to FIG. 13A, the bonding points 200 have a generally oblong shape, such as oval-elliptical, rectangular, rod or bar shaped, or the like. In some embodiments, the patterned roll 116a may comprise a combination of oblong shaped bonding points and circular/square/diamond shaped bonding points, such as an engraved roll used to form the bonding pattern shown in FIGS. 7A-7D.

In an oblong shaped bonding point, the bonding point 200 includes a major bonding point axis 230 and a minor bonding point axis 232 in which the major bonding point axis is of greater length than the minor bonding point axis.

In certain embodiments, the major axis of the bonding points in the same array (e.g., first annular array AA1) are oriented/aligned in the same direction while the major axis of the individual bonding points in the array forming the second member (e.g., second annular array AA2) of the array pair are oriented/aligned in an alignment that is oriented about 85° to 95° relative to the alignment of the major axis of bonding points 200 in the first annular array AA1. In certain embodiments, the individual bonding points in first annular array are oriented/aligned in an alignment that is oriented about 86° to 94°, such as from about 87° to 93°, 88° to 92°, 86° to 94°, 89° to 91°, or 90° relative to the alignment of the major axis of bonding points 200 in the second annular array AA2.

In certain embodiments, the bonding point density (number of individual bonding points per square centimeter on the surface of the patterned roll) is from about 50 to 65 cm²,

Referring back to FIG. 13A, a perspective view of a raised bonding point 200 in accordance with at least one embodiment of the invention is illustrated. As shown, the raised bonding point 200 includes a base 220, a raised surface 222, and a continuous sidewall 224 extending between the base 220 and raised surface 222. The raised surface is configured and arranged to engage the discrete regions of the surface of the nonwoven fabric and apply sufficient pressure and heat to cause fibers engaged with the bonding point to soften and fuse together to form an individual bond point on the surface of the nonwoven fabric.

In certain embodiments, the average length of the major bonding point axis 230 is from about 0.65 mm to 1.25 mm, and in particular, from about 0.70 to 1.20 mm, and more particularly, from about 1.15 to 0.72 mm, and even more particularly, from about 0.74 to 1.10 mm.

In a preferred embodiment, the average length of the major bonding point axis 230 from about 0.74 to 0.78 mm, with an average length that is about 0.76 mm being somewhat more preferred.

In certain embodiments, the average length of the major bonding point axis 230 is less than 1.25 mm, less than 1.24 mm, less than 1.23 mm, less than 1.22 mm, less than 1.21 mm less than 1.20 mm, less than 1.19 mm, less than 1.18 mm, less than 1.17 mm, less than 1.16 mm, less than 1.15 mm, less than 1.14 mm, less than 1.13 mm, less than 1.12 mm, less than 1.11 mm, less than 1.10 mm, less than 1.09 mm, less than 1.08 mm, less than 1.07 mm, less than 1.06 mm, less than 1.05 mm, less than 1.04 mm, less than 1.03 mm, less than 1.02 mm, less than 1.01 mm, less than 1.00 mm, less than 0.99 mm, less than 0.98 mm, less than 0.97 mm, less than 0.96 mm, less than 0.95 mm, less than 0.94 mm, less than 0.93 mm, less than 0.92 mm, less than 0.91 mm, less than 0.90 mm, less than 0.89 mm, less than 0.88 mm, less than 0.87 mm, less than 0.86 mm, less than 0.85 mm, less than 0.84 mm, less than 0.83 mm, less than 0.82 mm, less than 0.81 mm, less than 0.80 mm, less than 0.79 mm, less than 0.78 mm, less than 0.77 mm, less than 0.76 mm, less than 0.75 mm, less than 0.74 mm, less than 0.73 mm, less than 0.73 mm, less than 0.71 mm, less than 0.70 mm, less than 0.69 mm, less than 0.68 mm, less than 0.67 mm, or less than 0.66 mm.

In certain embodiments, the average length of the major bonding point axis 230 is greater than 0.65 mm, greater than 0.66 mm, greater than 0.67 mm, greater than 0.68 mm, greater than 0.69 mm, greater than 0.70 mm, greater than 0.71 mm, greater than 0.72 mm, greater than 0.73 mm, greater than 0.74 mm, greater than 0.75 mm, greater than 0.76 mm, greater than 0.77 mm, greater than 0.78 mm, greater than 0.79 mm, greater than 0.80 mm, greater than 0.81 mm, greater than 0.82 mm, greater than 0.83 mm, greater than 0.84 mm, greater than 0.85 mm, greater than 0.86 mm, greater than 0.87 mm, greater than 0.88 mm, greater than 0.89 mm, greater than 0.90 mm, greater than 0.91 mm, greater than 0.92 mm, greater than 0.93 mm, greater than 0.94 mm, greater than 0.95 mm, greater than 0.96 mm, greater than 0.97 mm, greater than 0.98 mm, greater than 0.99 mm, greater than 1.0 mm, greater than 1.01 mm, greater than 1.02 mm, greater than 1.03 mm, greater than 1.04 mm, greater than 1.05 mm, greater than 1.06 mm, greater than 1.07 mm, greater than 1.08 mm, greater than 1.09 mm, greater than 1.1 mm, greater than 1.11 mm, greater than 1.12 mm, greater than 1.13 mm, greater than 1.14 mm, greater than 1.15 mm, greater than 1.16 mm, greater than 1.17 mm, greater than 1.18 mm, greater than 1.19 mm, greater than 1.20 mm, greater than 1.21 mm, greater than 1.22 mm, greater than 1.23 mm, greater than 1.24 mm.

In certain embodiments, the average width of the minor bonding point axis 232 is from about 0.24 mm to 0.60 mm, and in particular, from about 0.25 to 0.55 mm, and more particularly, from about 0.27 to 0.50 mm, and even more particularly, from about 0.28 to 0.48 mm.

In preferred embodiments, the average width of the minor bonding point axis 232 is from about 0.24 to 0.36 mm, and in particular, from about 0.26 to 0.32 mm, and more particularly, from about 0.28 to 0.31 mm. In a slightly more preferred embodiment, the individual bond points have an average width that is about 0.30 mm.

In certain embodiments, the average width of the minor bonding point axis 232 is less than 0.6 mm, less than 0.59 mm, less than 0.58 mm, less than 0.57 mm, less than 0.56 mm less than 0.55 mm, less than 0.54 mm, less than 0.53 mm, less than 0.52 mm, less than 0.51 mm, less than 0.50 mm, less than 0.49 mm, less than 0.48 mm, less than 0.47 mm, less than 0.46 mm, less than 0.45 mm, less than 0.44 mm, less than 0.43 mm, less than 0.42 mm, less than 0.41 mm, less than 0.40 mm, less than 0.39 mm, less than 0.38 mm, less than 0.37 mm, less than 0.36 mm, less than 0.35 mm, less than 0.34 mm, less than 0.33 mm, less than 0.32 mm, less than 0.31 mm, less than 0.30 mm, less than 0.29 mm, less than 0.28 mm, less than 0.27 mm, less than 0.26 mm, or less than 0.25 mm.

In certain embodiments, the average width of the minor bonding point axis 232 is greater than 0.24 mm, greater than 0.25 mm, greater than 0.26 mm, greater than 0.27 mm, greater than 0.28 mm, greater than 0.29 mm, greater than 0.30 mm, greater than 0.31 mm, greater than 0.32 mm, greater than 0.33 mm, greater than 0.34 mm, greater than 0.35 mm, greater than 0.36 mm, greater than 0.37 mm, greater than 0.38 mm, greater than 0.39 mm, greater than 0.40 mm, greater than 0.41 mm, greater than 0.42 mm, greater than 0.43 mm, greater than 0.44 mm, greater than 0.45 mm, greater than 0.46 mm, greater than 0.47 mm, greater than 0.48 mm, greater than 0.49 mm, greater than 0.50 mm, greater than 0.51 mm, greater than 0.52 mm, greater than 0.53 mm, greater than 0.54 mm, greater than 0.55 mm, greater than 0.56 mm, greater than 0.57 mm, greater than 0.58 mm, or greater than 0.59 mm.

In the preparation of the nonwoven fabric of FIGS. 8A-8C, the major bonding point axis 230 is from about 1.0 to 1.2 mm, and in particular, from about 1.2 to 1.18 mm, and more particularly, from about 1.12 to 1.06 mm. In a slightly more preferred embodiment for the preparation of nonwoven fabrics of FIGS. 8A-8C, the individual bond points have an average length that is about 1.09 mm.

In the preparation of the nonwoven fabric of FIGS. 8A-8C, the minor bonding point axis 232 is from about 0.35 to 0.6 mm, and in particular, from about 0.4 to 0.5 mm, and more particularly, from about 0.42 to 0.52 mm. In a slightly more preferred embodiment for the preparation of nonwoven fabrics of FIGS. 8A-8C, the individual bond points have an average width that is about 0.47 mm.

In certain embodiments, the surface area of raised surface 222 is from about 0.15 to 0.75 mm², and in particular, from about 0.18 to 0.55 mm², and more particularly, from about 0.20 to 0.35 mm². In a preferred embodiment, the surface area of raised surface 222 is from about 0.20 to 0.30 mm², and somewhat more preferably, from about 0.21 to 0.25 mm².

In the preparation of the nonwoven fabric of FIGS. 8A-8C, the surface area of raised surface 222 is from about 0.25 to 0.55 mm², and in particular, from about 0.30 to 0.50 mm², and more particularly, from about 0.35 to 0.45 mm². In a preferred embodiment, the surface area of raised surface 222 is from about 0.38 to 0.42 mm², and somewhat more preferably, from about 0.4 mm².

FIGS. 13B and 13C are side views of a bonding point 200 taken along major axis 230 and minor axis 232 of the bonding point, respectively. In certain embodiments, the bonding points 200 may have an average height h (distance between the base 220 and raised surface 222) that is from about 0.5 to 1.25 mm, and in particular, about 0.85 to 1.15 mm, and more particularly, from about 0.9 to 1.1 mm, with a height from about 0.95 to 1.05 mm being somewhat more preferred, and a height of about 1.0 mm being even more preferred.

In certain embodiments, the average height of the bonding points 200 is less than 1.20 mm, less than 1.19 mm, less than 1.18 mm, less than 1.17 mm, less than 1.16 mm, less than 1.15 mm, less than 1.14 mm, less than 1.13 mm, less than 1.12 mm, less than 1.11 mm, less than 1.10 mm, less than 1.09 mm, less than 1.08 mm, less than 1.07 mm, less than 1.06 mm, less than 1.05 mm, less than 1.04 mm, less than 1.03 mm, less than 1.02 mm, less than 1.01 mm, less than 1.00 mm, less than 0.99 mm, less than 0.98 mm, less than 0.97 mm, less than 0.96 mm, less than 0.95 mm, less than 0.94 mm, less than 0.93 mm, less than 0.92 mm, less than 0.91 mm, less than 0.90 mm, less than 0.89 mm, less than 0.88 mm, less than 0.87 mm, less than 0.86 mm, less than 0.85 mm, less than 0.84 mm, less than 0.83 mm, less than 0.82 mm, less than 0.81 mm, less than 0.80 mm, less than 0.79 mm, less than 0.78 mm, less than 0.77 mm, less than 0.76 mm, less than 0.75 mm, less than 0.74 mm, less than 0.73 mm, less than 0.73 mm, less than 0.71 mm, less than 0.70 mm, less than 0.69 mm, less than 0.68 mm, less than 0.67 mm, less than 0.66 mm, less than 0.65 mm, less than 0.64 mm, less than 0.63 mm, less than 0.62 mm, less than 0.61 mm, less than 0.60 mm, less than 0.59 mm, less than 0.58 mm, less than 0.57 mm, less than 0.56 mm, less than 0.55 mm, less than 0.54 mm, less than 0.53 mm, less than 0.52 mm, or less than 0.51 mm.

In certain embodiments, the average height of the bonding points 200 is greater than 0.5 mm, greater than 0.51 mm, greater than 0.52 mm, greater than 0.53 mm, greater than 0.54 mm, greater than 0.55 mm, greater than 0.56 mm, greater than 0.57 mm, greater than 0.58 mm, greater than 0.59 mm, greater than 0.60 mm, greater than 0.61 mm, greater than 0.62 mm, greater than 0.63 mm, greater than 0.64 mm, greater than 0.65 mm, greater than 0.66 mm, greater than 0.67 mm, greater than 0.68 mm, greater than 0.69 mm, greater than 0.70 mm, greater than 0.71 mm, greater than 0.72 mm, greater than 0.73 mm, greater than 0.74 mm, greater than 0.75 mm, greater than 0.76 mm, greater than 0.77 mm, greater than 0.78 mm, greater than 0.79 mm, greater than 0.80 mm, greater than 0.81 mm, greater than 0.82 mm, greater than 0.83 mm, greater than 0.84 mm, greater than 0.85 mm, greater than 0.86 mm, greater than 0.87 mm, greater than 0.88 mm, greater than 0.89 mm, greater than 0.90 mm, greater than 0.91 mm, greater than 0.92 mm, greater than 0.93 mm, greater than 0.94 mm, greater than 0.95 mm, greater than 0.96 mm, greater than 0.97 mm, greater than 0.98 mm, greater than 0.99 mm, greater than 1.0 mm, greater than 1.01 mm, greater than 1.02 mm, greater than 1.03 mm, greater than 1.04 mm, greater than 1.05 mm, greater than 1.06 mm, greater than 1.07 mm, greater than 1.08 mm, greater than 1.09 mm, greater than 1.1 mm, greater than 1.11 mm, greater than 1.12 mm, greater than 1.13 mm, greater than 1.14 mm, greater than 1.15 mm, greater than 1.16 mm, greater than 1.17 mm, greater than 1.18 mm, greater than 1.19 mm, or greater than 1.20 mm.

In certain embodiments for the preparation of the nonwoven fabrics of FIGS. 8A-8C, the bonding points 200 may have an average height h (distance between the base 220 and raised surface 222) that is from about 0.5 to 0.85 mm, and in particular, about 0.55 to 0.8 mm, and more particularly, from about 0.6 to 0.75 mm, with a height from about 0.65 to 0.7 mm being somewhat more preferred, and a height of about 0.68 mm being even more preferred.

In certain embodiments, the continuous sidewall 224 of the bonding point 200 may be perpendicular to the surface 202 of the patterned roll. In certain other embodiments, the continuous sidewall 224 of the bonding point 200 may be inclined relative to the surface of the patterned roll. As shown in FIGS. 13B and 13C, angle a7 between the surface 202 and the continuous sidewall 224 may be from about 90° to 120°, with an angle a7 from about 90° to 120°, and in particular, from about 105 to 115°.

In certain other embodiments, the cross direction arrays (e.g., CDA1, CDA2) extending in the CD of the patterned roll 116a may be angled relative to the central axis 210. That is, rather than being parallel to the central axis, the cross direction arrays may extend at an angle of inclination that is greater than 0° and less than 6° relative to a horizontal line (the horizontal line being parallel to the central axis 210) drawn laterally across the surface of the patterned roll. In particular, the angle of inclination may range from greater than 0° to less than 6°. In a preferred embodiment, the angle of inclination is from about 0.5° to 4°, and in particular, from about 1° to 3°, with an angle of about 2° being preferred.

Nonwoven fabrics in accordance with embodiments of the present invention may be used in a wide variety of applications, such as absorbent and personal hygiene applications, medical applications, such as clothing, gowns, face masks, wound dressings, and the like. Other applications include industrial applications, such as home construction, filtration, furniture, and the like. Other possible applications may include agricultural, such as ground coverings, root wraps, plant coverings, protective bags, and the like. In one embodiment, the nonwoven fabric may be combined with one or more additional layers to form a laminate. In particular, a laminate comprising the inventive nonwoven fabrics previously discussed may be adapted for use in a disposable absorbent article such as a diaper, a pant, an adult incontinence product, a sanitary napkin or any other article that may benefit from the desirable properties provided with the nonwoven fabrics in accordance with embodiments of the present invention.

Nonwoven fabrics in accordance with embodiments of the invention may be used to prepare a variety of different structures. For example, in some embodiments, the inventive bonded nonwoven fabric may comprise from about 1 to 10 layers, and in particular, 2 to 8 layers, such as from 3 to 6 layers.

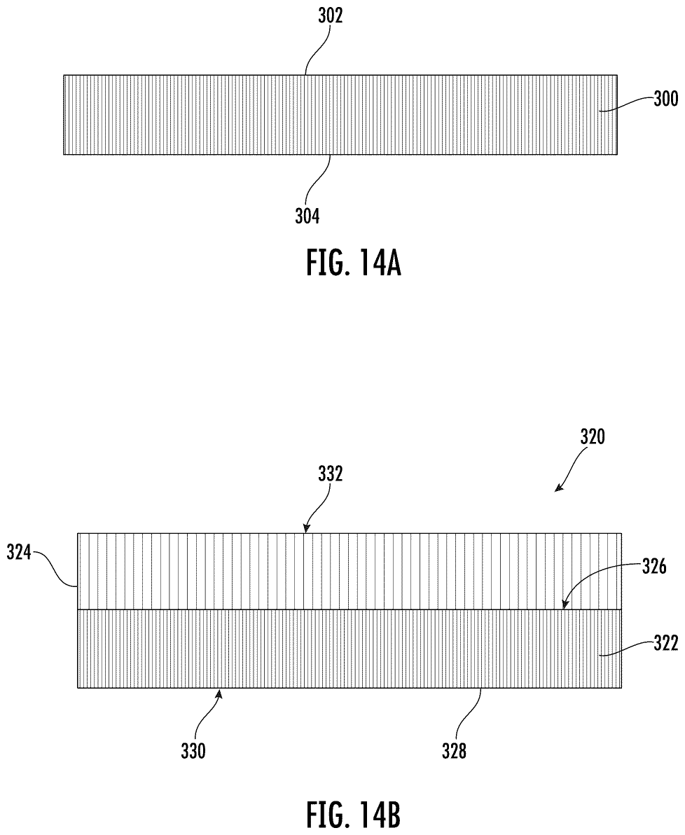

In this regard, FIG. 14A is a cross-sectional side view of a single layer bonded nonwoven fabric 300 in accordance with at least one embodiment of the invention. Nonwoven fabric 300 comprises a plurality of fibers that are bonded together with a plurality of spaced apart individual bond points to form a coherent web. Nonwoven fabric 300 comprises a first surface 302 and a second surface 304 in which at least one of the surfaces includes a bond pattern in accordance with the one or more of the previously discussed bond patterns. In certain embodiments, first surface 302 includes a bond pattern in accordance with at least one embodiment of the invention, and second surface does not include a bond pattern on the surface thereof. In other words, during bonding only surface 302 is contacted by a patterned roll having a plurality of individual bonding points.

With reference to FIG. 14B, a cross sectional side view of a two layer composite nonwoven in accordance with at least one embodiment of the invention is illustrated and broadly designated by reference character 320. Composite nonwoven fabric 320 includes a first layer 322 and a second layer 324 that are joined to each other at interface 326. First layer 322 comprises a surface 328 having a bond pattern 330 disposed thereon in accordance with at least one embodiment of the disclosure as discussed previously. Second nonwoven fabric 324 includes outer surface 332. In certain embodiments, outer surface 332 of the second nonwoven fabric layer 324 may comprise a substantially smooth surface that has not been subjected to point bonding via a calender roll. In other embodiments, outer surface 332 may include a bond pattern imparted thereon in accordance with at least one embodiment of the disclosure as discussed previously.

The second nonwoven layer may comprise the same type of nonwoven fabric as the first nonwoven layer. In certain embodiments, the second nonwoven layer may comprise the same type of nonwoven fabric as the first nonwoven layer. For example, the second nonwoven layer may be a meltblown fabric, a spunbond fabric, a carded fabric, an air laid fabric, a resin bonded fabric, a spunlace fabric, or the like.

In some embodiments, both the first nonwoven fabric and the second nonwoven fabric may each comprise a spunbond nonwoven fabric.

In certain embodiments, the bonded nonwoven fabric may be combined with one or more additional layers to prepare a composite or laminate material.

Examples of such composites/laminates may include a spunbond composite, such as a spunbond-meltblown (SM) composite, a spunbond-meltblown-spunbond (SMS) composite, or a spunbond-meltblown-meltblown-spunbond (SMMS) composite. In some embodiments, composites may be prepared comprising a layer of the bonded nonwoven fabric and one or more film layers. It should be recognized other configurations are also in the scope of the invention.

In these multilayer structures, the basis weight of the spunbond nonwoven fabric layer may range from as low as 5 g/m² and up to 150 g/m². In some embodiments comprising a multilayer structure (e.g., SM, SMS, and SMMS), the amount of the meltblown in the composite structure may range from about 5 to 30 weight %, and in particular, from about 5 to 15 weight % of the structure as a weight percentage of the structure as a whole.

Multilayer structures in accordance with embodiments can be prepared in a variety of manners including continuous in-line processes where each layer is prepared in successive order on the same line, or depositing a second nonwoven layer on a previously formed spunbond layer. The layers of the multilayer structure can be thermally bonded together to form a multilayer composite sheet material to provide a composite sheet material having the bonding patterns described herein. In addition, composite sheet materials in accordance with certain embodiments of the invention may also be subjected to other bonding techniques, such as thermal bonding via an air through dry, mechanical bonding, adhesive bonding, hydroentangling, or combinations of these. In certain embodiments, the layers are thermally point bonded to each other by passing the multilayer structure through a bonding unit comprising a pair of calender rolls in which the patterned roll of the calender has an engraved surface comprising the inventive bonding pattern thereon.

As previously noted, fabrics prepared in accordance with embodiments of the invention may be used in wide variety of articles and applications. For instance, embodiments of the invention may be used for personal care applications, for example products for babycare (diapers, wipes), for femcare (pads, sanitary towels, tampons), for adult care (incontinence products), or for cosmetic applications (pads), agricultural applications, for example root wraps, seed bags, crop covers, industrial applications, for example work wear coveralls, airline pillows, automobile trunk liners, sound proofing, and household products, for example mattress coil covers and furniture scratch pads.

The following examples are provided for illustrating one or more embodiments of the present invention and should not be construed as limiting the invention.

### EXAMPLES

Spunbond nonwoven fabrics in the following examples were prepared with a Reicofil 4S spunbond spinning line produced by Reifenhaeuser. Unless otherwise indicated all percentages are weight percentages. The materials and test methods used in the examples are identified below.

### Test Methods:

Basis weight was measured in accordance with NWSP 130.1.

MD and CD tensile strengths were measure in accordance with NWSP 110.4B (modified: gauge length was 100 mm, sample width was 50 mm, and speed was 100 mm/min.).

MD and CD elongations were measure in accordance with NWSP 110.4B (modified: gauge length was 100 mm, sample width was 50 mm, and speed was 100 mm/min.).

Caliper was measure in accordance with NWSP 120.6.

Air Permeability was measured in accordance with ASTM 90.3.

Hydrohead was measured in accordance with WSP80.6.

Handle-O-Meter was measure in accordance with NWSP 90.3.

Softness Bending was measured in accordance with WSP90.1.

Abrasion Resistance was measured in accordance with NWSP 20.5: The surface of each test sample of the nonwoven fabric was subjected to 32 rubs at a pressure of 9 KPa with a white rubber. The test sample was weighed prior to being rubbed. Following rubbing, the sample was shaved and then reweighed again.

Martindale Abrasion Test Method Grading Scale (Martindale Abrasion Scoring). In this method, the surface of each test sample of the nonwoven fabric was subjected to 32 rubs at a pressure of 9 KPa with a white rubber. The surface of the samples were then evaluated under a microscope for the appearance of defects and then graded on a scale of 1-5, with a score of 1-2 being considered acceptable to the consumer, and a score of 3-5 being unacceptable. The lower the score, the better the abrasion resistance of the fabric. Scores are determined based on the following criteria:
Score between 1 and 2: samples exhibit little to no visible defects (the sample may include fuzziness (short fibers lifted from the fabric) and micro-pills (less than 2 mm in diameter);
Score between 2 and 3: samples exhibit pills (fibers roll into ball measuring less than 2 mm in diameter), small strings (less than 2 mm wide), and the pills and strings are not connected to a network of individual fibers;
Scores of 3 and above: samples will exhibit ropes (long fibers that twist to form a braid rope), loft, spider webs (networks of interconnected ropes and pills that increase the loft of the fabric, and sever spider webs, holes in the fabric surface, and increased loft.

### Materials:

“PP-1” refers to a homopolymer polypropylene having an MFR of 34 g/10 min available from Braskem under the product number CP360H.

“PP-2” refers to a homopolymer polypropylene having an MFR of 1284 g/10 min available from Braskem under the product number H155.

“PP-3” refers to a metallocene catalyzed polypropylene having a MFR of 35 g/10 min available from Basell under the product number HM 562S.

“PP-4” refers to a homopolymer polypropylene having a MFR of 35 g/10 min available from IRPC under the product code 1105SC.

“CoPP’ refers to a polypropylene copolymer having an MFR of 48 g/10 min available from Exxon Mobil under the tradename VISTAMAXX™ 7050FL.

“L-MODU” refers to a low isotacticity polypropylene copolymer are available from Idemitsu under the product name L-MODU™.

“TiO₂” refers to Remafin White PPF2K002G titanium dioxide available from Clariant/Avient under the product code PP0N420701.

“SA” refers to a slip agent available from Evonik Industries under the tradename ACCUREL® SF 617.

### Comparative Example 1

In Comparative Example 1 a two-layer spunbond nonwoven fabric was prepared in which the first layer comprised non-crimped filaments composed of PP-1, and the second layer comprised bicomponent filaments having a side-by-side (70:30) configuration in which one side comprised PP-1 and the other side comprised a blend of PP-1 and PP-2. The amount of PP-1 in the blend was 98.65 weight percent, and amount of PP-2 was 1.35 weight percent, based on the total weight of the blend.

The resulting two layer spunbond fabric was thermally point bonded with a calender bonding unit comprising a smooth anvil roll and an engraved pattern roll with a bond pattern similar to that shown in FIG. 1, with the exception of the size of the individual bond points, bond point density, and the overall resulting bonded % surface area of the bonded nonwoven fabric. The patterned roll was heated to a temperature of approximately 152° C. The percentage of the surface of the nonwoven fabric bonded in Comparative Example 1 was 18.1%, and the bond density was 49.9 bonding points per cm².

Inventive Example 1 was identical to the nonwoven fabric of Comparative Example 1 with the exception that the nonwoven fabric sample was thermally pointed bonded with a calender bonding unit having a patterned roll with a bonding pattern in accordance with the pattern shown in FIG. 1. In addition, the patterned roll had a cross directional inclination of approximately 2°. The calender bonding unit was operated at a temperature of approximately 155° C. The percentage of the surface of the nonwoven fabric bonded in Inventive Example 1 was 9.92%, and the bond density was 55.2 bonding points per cm².

The dimensions and physical properties of the engraving rolls used in Comparative Example 1 and Inventive Example 1 are summarized in Tables 1 and 2, below.

TABLE 1
Dimensions of Bonding Points of Comparative
Example 1 and Inventive Example 1

| | Bond point | Bond point | Bond point | Bond point |
|---|---|---|---|---|
| | length* | Width** | surface area | density (bond |
| Example No. | (mm) | (mm) | (mm²) | points/cm²) |
| Comparative | 0.882 | 0.524 | 0.36 | 49.9 |
| Example 1 | | | | |
| Inventive | 0.76 | 0.30 | 0.18 | 55.2 |
| Example 1 | | | | |

*Measurement of major axis of bonding point.
**Measurement of major axis of bonding point.

TABLE 2
Engraving Roll Dimensions of Comparative Example 1 and Inventive Example 1

| | | Distance | Distance | | |
|---|---|---|---|---|---|
| | Distance between oval | between oval | between bond | Bonded | Angle between |
| | bond mid points in | bond midpoints | midpoints in | Surface area | DD array and |
| Example No. | CD* (mm) | in MD* (mm) | DD* (mm) | (%) | CD array (°) |
| Comparative | 2.634 | 1.521 | 1.521 | 18.1 | 30° |
| Example 1 | | | | | |
| Inventive | 1.9 | 1.9 | 1.34 | 9.92 | 45° |
| Example 1 | | | | | |

*Measurements between adjacent bonding points in same array.

10 samples of Comparative Example 1 and Inventive Example 1 were prepared and evaluated. The average results are provided in Tables 3-5 below.

TABLE 3
Nonwoven Fabric Bonding Properties

| | Average | | | |
|---|---|---|---|---|
| | collective | | | |
| | distance | Average | | |
| | between | surface | Average | |
| | adjacent | area of | Bond | Percentage |
| | bond | bond | Point | surface |
| Example | points | points | Packing | area |
| No. | (mm) | (mm²) | (mm⁻¹) | bonded |
| Comparative | 1.19 | 0.36 | 3.30 | 18.1 |
| Example 1 | | | | |
| Inventive | 1.28 | .18 | 7.11 | 9.92 |
| Example 1 | | | | |

TABLE 4
Physical/Mechanical properties of Comparative Example 1 and Inventive Example 1

| | | | MD | | | | |
|---|---|---|---|---|---|---|---|
| | Basis | | tensile | CD tensile | MD | CD | Air |
| Example | weight | Caliper | strength | strength | Elongation | Elongation | Permeability |
| No. | (gsm) | (mm) | (N/cm) | (N/cm) | (%) | (%) | (m³/m²/min) |
| Comparative | 25.32 | 0.26 | 7.21 | 3.86 | 65.44 | 76.04 | 153.73 |
| Example 1 | | | | | | | |
| Inventive | 24.7 | 0.27 | 8.13 | 4.16 | 68.46 | 86.60 | 150.77 |
| Example 1 | | | | | | | |
| Percent | n/a | n/a | 12.76 | 20.37 | 4.62 | 13.89 | −1.93 |
| Increase | | | | | | | |

From Table 4, it can be seen that the Inventive Nonwoven fabrics exhibited increases in both tensile strengths and percent elongations in comparison to Comparative Example 1. The increases in tensile strengths were particularly surprising as increases in % bonding area would typically be expected to also increase the tensile strength of the nonwoven fabric in comparison to a nonwoven fabric having less % bond area.

In particular, the nonwoven fabric of Inventive Example 1 exhibited an increase in machine direction tensile strength of greater than 10%, and in particular, greater than 12%, in comparison to the nonwoven fabric of Comparative Example 1. In addition, the nonwoven fabric of Inventive Example 1 exhibited an increase in cross direction tensile strength of greater than 15%, and in particular, greater than 20%, in comparison to the nonwoven fabric of Comparative Example 1.

Machine and cross direction percent elongations were also improved in the nonwoven fabric of Inventive Example 1 in comparison to the nonwoven fabric of Comparative Example 1. For example, the nonwoven fabric of Inventive Example 1 exhibited an increase of 4.6% and 13.9% in machine and cross direction elongation %, respectively, in comparison to the nonwoven fabric of Comparative Example 1.

Air permeability between the fabrics of Comparative Example 1 and Inventive Example 1 were comparable, with Inventive Example 1 showing a negligible decrease of less than 2%.

TABLE 5
Abrasion and Softness for Comparative Example and Inventive Example 1

| | Martindale | Abrasion | Abrasion | Average | CD | MD | Average |
|---|---|---|---|---|---|---|---|
| Example | Abrasion | Smooth | engraved | Abrasion | H-O-M | H-O-M | H-O-M |
| No. | (Score) | (mg) | (mg) | (mg) | (g) | (g) | (g) |
| Comparative | 1.75 | 20.50 | 3.1 | 11.8 | 7.38 | 4.06 | 5.72 |
| Example 1 | | | | | | | |
| Inventive | 1.43 | 5.30 | 3.4 | 4.35 | 6.81 | 3.75 | 5.28 |
| Example 1 | | | | | | | |
| Increase (%) | −18.23 | −74.15 | 9.68 | −63.14 | −7.72 | −7.64 | −8.65 |
| Percent | 20.1 | 118 | 9.2 | 92 | 8 | 8 | 8 |
| Difference (%) | | | | | | | |

Surprisingly, the Inventive nonwoven fabric also demonstrated improvements in abrasion resistance and softness in comparison to the Comparative Example 1. In particular, a fabric having a higher % bonded area would be expected to have reduced abrasion resistance in comparison to the nonwoven fabric of Inventive Example 1, which had a % bonded area that was approximately half that of the nonwoven fabric of Comparative Example 1. The smooth side of Inventive Example 1 (the side in contact with the surface of the smooth anvil roll) exhibited a decrease of about 74% of material removed during the abrasion test in comparison to the nonwoven fabric of Comparative Example 1. The surface of the nonwoven fabric of Inventive Example 1 in contact with the pattern roll did exhibit a slight percent increase in material removed during abrasion testing (about 9.7%) in comparison to the nonwoven fabric of Comparative Example 1; however, the average abrasion resistance of the two sides of the nonwoven fabric exhibited a decrease of approximately 63% of material removed during abrasion resistance in comparison to the nonwoven fabric of Comparative Example 1. Accordingly, the results show that the inventive fabrics, having a reduction in percent bond area in comparison to the comparative nonwoven fabrics (having a higher % bonded area) exhibited improvements in abrasion resistance.

Evaluation with the Martindale Scoring Method also showed that the inventive fabrics had improved abrasion resistance in comparison to the nonwoven fabric of Comparative Example 1. In particular, Inventive Example 1 exhibited a percent decrease in Martindale Score of 18.2%, which reflects a nearly 20% improvement in abrasion resistance. Again, this is surprising as the amount of surface area bonded in Inventive Example 1 is nearly half of that of Comparative Example 1.

In addition, the inventive fabrics also exhibited improvements with respect to softness in comparison to the nonwoven fabrics of Comparative Example 1 as evidenced by the reduction in Handle-O-Meter (H-O-M) values.

From the foregoing results, it is evident that the inventive nonwoven fabrics exhibit higher mechanical properties in combination with increased abrasion resistance and increased softness. Such improvements are not expected in combination as one would expect a reduction in both tensile strengths and abrasion resistance as the % of bonded area of the nonwoven fabric is reduced in comparison to Comparative Example 1.

### Inventive Examples 2 and 3

In Inventive Examples 2 and 3, a three layer spunbond nonwoven fabric was prepared in which each layer comprised the same polymeric blend. The nonwoven fabric was thermally point bonded with a calender bonding unit was operated at a temperature of approximately 150° C. The calender bonding unit was the same engraving pattern of Inventive Example 1. The percentage of the surface of the nonwoven fabric bonded in Inventive Examples 2 and 3 was 9.92%, and the bond density was 55.2 bonding points per cm². The nonwoven of Inventive Example 2 was prepared at a polymer throughput of 180 kg/m/hr and a cabin pressure of 5100 Pa. The nonwoven of Inventive Example 3 was prepared at a polymer throughput of 160 kg/m/hr and a cabin pressure of 5300 Pa.

The fibers of Inventive Examples 2 and 3 comprised a blend of 92.2% PP-3, 7.0% CoPP, and 0.8% of TiO₂ and SA.

### Inventive Example 4

In the following Inventive Example 4, a three layer spunbond nowoven fabric was prepared with a calender bonding unit comprising a smooth anvil roll and an engraved pattern roll with a bond pattern configured to produce a bond pattern similar to that shown in FIG. 8A. The die cabin pressure was operated a pressures above 5,100 Pa with a polymer through put of 200 kg/m/hour.

The fibers of Inventive Example 4 comprised a blend of 81.4% PP-4, 18.0% L-MODU, and 0.8% of TiO₂ and SA. The calender bonding unit was operated at a temperature of approximately 150° C. The percentage of the surface of the nonwoven fabric bonded in Inventive Example 4 was 13.4%, and the bond density was 33.4 bonding points per cm².

The average height of the individual bonding points on the engraving roll was 68 mm, the average width was 0.47 mm, and the average length of each bonding point was 1.09 mm. The average surface area of the bonding points was 0.4 mm², with a collective average bond distance of 1.6 mm, and a bond packing density of 4 mm⁻¹.

The properties of the nonwoven fabric of Inventive Examples 2-4 are provided in the Table 6, below.

TABLE 6
MECHANICAL PROPERTIES OF INVENTIVE EXAMPLES 2-4

| | | | MD | | | | |
|---|---|---|---|---|---|---|---|
| | | | tensile | CD tensile | | | |
| | Basis | Filament | strength | strength | MD | CD | Air |
| Example | weight | Size | (N/25.4 | (N/25.4 | Elongation | Elongation | Permeability |
| No. | (gsm) | (Dtex) | mm) | mm) | (%) | (%) | (m³/m²/s) |
| Inventive | 15 | 1.26 | 17.3 | 7.2 | 58.4 | 76.7 | 83.3 |
| Example 2 | | | | | | | |
| Inventive | 15.1 | 0.9 | 19.3 | 9.1 | 60.4 | 77.4 | 78.1 |
| Example 3 | | | | | | | |
| Inventive | 15 | 1.1-1.3 | 15.2 | 8.6 | 79.9 | 93.5 | 86 |
| Example 4 | | | | | | | |

TABLE 7
BENDING SOFTNESS AND HYDROHEAD
INVENTIVE EXAMPLES 2-4

| | Basis | Filament | MD | CD | Average | Hydro- |
|---|---|---|---|---|---|---|
| Example | weight | Size | Bending | Bending | Bending | head |
| No. | (gsm) | (Dtex) | (mm) | (mm) | (mm) | (mm H₂0) |
| Inventive | 15 | 1.26 | 45.5 | 20.2 | 32.8 | 107.2 |
| Example 2 | | | | | | |
| Inventive | 15.1 | 0.9 | 43.4 | 20.2 | 31.8 | 110.7 |
| Example 3 | | | | | | |
| Inventive | 15 | 1.1-1.3 | 42.1 | 27.9 | 35.0 | 99.9 |
| Example 4 | | | | | | |

Interestingly, it was observed that a reduction in throughput and an increase in cabin pressure resulted in finer fibers as can be seen in comparison of Inventive Examples 2 and 3. In particular, the fibers of Inventive Example 2 exhibited an average filament size of 1.26 Dtex whereas the fibers of Inventive Example 3 exhibited an average filament size of 0.9 Dtex. This represent a decrease of 28.6% in fiber fineness.

1. A nonwoven fabric comprising a plurality of fibers bonded with a bond pattern on a surface thereof to form a coherent web, the nonwoven fabric having a vertical axis extending in a machine direction and a horizontal axis extending in a cross direction, the bond pattern comprising a plurality of spaced apart pairs of arrays extending in the machine, cross, and diagonal directions of the nonwoven fabric, wherein each array comprises a plurality of spaced apart bond points having an oblong shape, and wherein the nonwoven fabric has a percent bonded surface area less than about 12% and an average bond point packing value from about 6.5 to 8.0 mm⁻¹.

2. The nonwoven fabric according to claim 1, wherein the nonwoven fabric wherein the bond points have an average surface area from about 0.15 to 0.25 mm² and a bond point density from about 50 to 60 individual bond points per square centimeter.

3. The nonwoven fabric according to claim 1, wherein the nonwoven fabric has a Martindale Abrasion Score from about 1.0 to 1.5, a cross direction Handle-o-Meter from about 6.6 to 7.2 grams, a machine direction Handle-O-Meter from about 3.5 to 3.95 grams, and an average abrasion resistance as determined by the weight of removed material from 3.2 to 5.5 grams.

4. The nonwoven fabric according to claim 1, wherein a percent bonded surface area of the nonwoven fabric is from about 9.8 to 10%, the average bond point surface area is from about 0.16 to 0.2 mm², the average bond point packing value is from about 6.75 to 7.25 mm-1, and a bond point density of the nonwoven fabric is from about 52 to 58 individual bond points per square centimeter.

5. The nonwoven fabric according to claim 1, wherein the nonwoven fabric exhibits a Martindale Abrasion Score of 1 to 2, a cross direction Handle-o-Meter from about 6.7 to 7.0 grams, a machine direction Handle-O-Meter from about 3.6 to 3.9 grams, and an average abrasion resistance as determined by the weight of removed material from 3.4 to 3.6 grams.

6. The nonwoven fabric according to claim 1, wherein the bond pattern comprises alternating first and second arrays of individual bond points extending in the cross direction of the nonwoven fabric, wherein the individual bond points of said first array and second array each have a length and a width, and wherein the lengths of the individual bond points of the first array are aligned in substantially the same direction and at an angle that is from about 43 to 47 degrees relative to the horizontal axis of the nonwoven fabric, and the lengths of the individual bond points of the second array are rotated from about 88 to 92 degrees relative to the alignment of the lengths of the individual bond points of the first array, and wherein the individual bond points of the second array are offset in the cross direction from adjacent individual bond points of the first array.

7. The nonwoven fabric according to claim 1, wherein the nonwoven fabric has one or more of the following properties:
a number of individual bond points per cm² is from about 45 to 60;
a percent bonded area of the nonwoven fabric from about 9 to 10.5%;
a distance between adjacent bonds in the cross direction is from about 1.4 to 1.6 mm; and
a distance between adjacent bond points in a same array in a diagonal direction of the nonwoven fabric is from about 0.7 to 0.95 mm.

8. The nonwoven fabric according to claim 1, wherein the bond points have an oval-elliptical shape, rectangular shape, rod shape, or a combination thereof.

9. The nonwoven fabric according to claim 1, wherein the nonwoven fabric exhibits a Martindale Abrasion Score of less than 1.5.

10. The nonwoven fabric according to claim 1, wherein the nonwoven fabric has a basis weight from about 20 to 30 gsm and exhibits one or more of a softness as demonstrated by a cross direction handle-o-meter of less than 7.0 grams or a softness as demonstrated by a machine direction handle-o-meter of less than 3.9 grams.

11. The nonwoven fabric according to claim 1, wherein the nonwoven fabric has a percent bonded area from about 9.6 to 10.4%, an average individual bond point surface area from about 0.15 to 0.25 mm², an average bond point packing value from about 6.75 to 7.25 mm⁻¹, a bond point density from about 50 to 60 individual bond points per square centimeter, a Martindale Abrasion Score from about 1.0 to 1.5, a cross direction Handle-o-Meter from about 6.6 to 7.2 grams, a machine direction Handle-O-Meter from about 3.5 to 3.95 grams, and an average abrasion resistance as determined by the weight of removed material from 3.2 to 5.5 grams.

12. The nonwoven fabric according to claim 1, wherein the nonwoven fabric has a percent bonded area from about 9.6 to 10.2%, an average individual bond surface area from about 0.15 to 0.25 mm², an average bond point packing value from about 6.75 to 7.25 mm-1, and an average increase in tensile strengths that are 10% greater, such as from about 10 to 50%, from about 12 to 24%, or from about 12 to 22%, in comparison to a similarly prepared nonwoven fabric having a bond point packing of less than 3.5 mm⁻¹.

13. The nonwoven fabric according to claim 1, wherein the nonwoven fabric has a percent bonded area from about 9.6 to 10.2%, an average individual bond surface area from about 0.15 to 0.25 mm², an average bond point packing value from about 6.75 to 7.25 mm-1, and one or more of the following properties:
i) an improvement in abrasion resistance as exemplified by a percent difference in the Martindale Abrasion Score of 10 to 30% in comparison to a similarly prepared nonwoven fabric having a bond point packing of less than 3.5 mm⁻¹;
ii) an average percent decrease in the weight of material removed during the abrasion testing (in accordance with Test Method NWSP 20.5) from 5 to 150%, such as from about 8 to 120% or 9 to 95% in comparison to a similarly prepared nonwoven fabric having a bond point packing of less than 3.5 mm⁻¹; and
iii) improvements in softness as demonstrated by an average improvement of Handle-O-Meter values from about 5 to 20%, such as from about 6 to 15% or 8 to 10%, in comparison to a similarly prepared nonwoven fabric having a bond point packing of less than 3.5 mm⁻¹.

14. The nonwoven fabric according to claim 1, wherein the nonwoven fabric has a percent bonded area from about 9.6 to 10.2%, an average individual bond surface area from about 0.15 to 0.25 mm², an average bond point packing value from about 6.75 to 7.25 mm⁻¹, and one or more of the following properties:
i) an improvement in abrasion resistance as exemplified by a percent difference in the Martindale Abrasion Score of 10 to 30% in comparison to a similarly prepared nonwoven fabric having a bond point packing of less than 3.5 mm⁻¹;
ii) an average percent decrease in the weight of material removed during the abrasion testing (in accordance with Test Method NWSP 20.5) from 5 to 150%, such as from about 8 to 120% or 9 to 95% in comparison to a similarly prepared nonwoven fabric having a bond point packing of less than 3.5 mm⁻¹;
iii) improvements in softness as demonstrated by an average improvement of Handle-O-Meter values from about 5 to 20%, such as from about 6 to 15% or 8 to 10%, in comparison to a similarly prepared nonwoven fabric having a bond point packing of less than 3.5 mm⁻¹;
iv) an increase in machine direction (MD) tensile strengths that are from about 10 to 50% greater, such as from about 10 to 30%, such as from about 12 to 20%, or from about 12 to 15% greater, in comparison to a similarly prepared nonwoven fabric having a bond point packing of less than 3.5 mm⁻¹;
v) an increase in cross direction (CD) tensile strengths that are from about 10 to 50% greater, such as from about 10 to 30%, such as from about 15 to 25%, from about 18 to 24%, or from about 19 to 21%, in comparison to a similarly prepared nonwoven fabric having a bond point packing of less than 3.5 mm⁻¹; and
vi) an increase in percent elongation that is from about 4 to 50%, such as from about 5 to 20%, or from about 5 to 15%, in comparison to a similarly prepared nonwoven fabric having a bond point packing of less than 3.5 mm⁻¹.

15. The nonwoven fabric according to claim 1, wherein the nonwoven fabric comprises at least two layers in which one of the layers is selected from the group of meltblown layer, carded fabric layer, spunbond layer, resin bonded layer, airlaid fabric layer, and a spunlace layer.

16. An absorbent article comprising the nonwoven fabric according to claim 1.

17. A composite sheet material comprising the nonwoven fabric according to claim 1.

18. A calender bonding unit for point bonding a sheet material, the calender bonding unit comprising a pair of cooperating cylindrical rolls in which at least one of the rolls includes an engraved pattern thereon, the engraved pattern comprising a plurality of individual, spaced apart bonding points that extend radially outward from a surface of the roll, the plurality of bonding points defining a pattern comprising a plurality of spaced apart arrays that extend in the cross and radial direction of the roll, and are configured and arranged to thermally point bond a nonwoven fabric in which a percent bonded surface area of the nonwoven fabric is less than about 12% and an average bond point packing value of the nonwoven fabric is from about 6.5 to 8 mm⁻¹, and wherein the engraved pattern includes one or more of the following features:
the engraved pattern further comprises a plurality of spaced apart pairs of arrays that extend circumferentially about the roll in a spiral-like shape;
the bonding points have a continuous side wall and a raised surface, the raised surfaces have an average surface area from about 0.15 to 0.25 mm²;
the number of bonding points is from about 50 to 60 individual bonding points per square centimeter; and
an average length of the bonding points is from about 0.74 to 0.78 mm, and an average width of the bonding points is from about 0.24 to 0.36 mm.

19. A system for preparing a nonwoven fabric comprising,
a first polymer source.
a spinbeam in communication with the first polymer source, the spinbeam configured and arranged to produce a plurality of polymer fibers;
a collection surface disposed below the spinbeam for depositing the plurality of polymer fibers thereon to form a web of fibers;
a thermal bonding unit disposed downstream of the spin beam, the thermal bonding unit comprising a pair of cooperating cylindrical rolls in which at least one of the rolls includes an engraved pattern thereon, the engraved pattern comprising a plurality of individual, spaced apart bonding points that extend radially outward from a surface of the roll, the plurality of bonding points defining a pattern comprising a plurality of spaced apart arrays that extend in the cross and radial direction of the roll, and are configured and arranged to thermally point bond the web of fibers to form a nonwoven fabric in which a percent bonded surface area of the nonwoven fabric is less than about 12% and an average bond point packing value of the nonwoven fabric is from about 6.5 to 8 mm⁻¹.

20. A Method of preparing a bonded nonwoven fabric comprising:
providing a nonwoven web comprising a plurality of fiber;
introducing the nonwoven web into a thermal bonding unit comprising a pair of cooperating cylindrical rolls in which at least one of the rolls includes an engraved pattern thereon, the engraved pattern comprising a plurality of individual, spaced apart bonding points that extend radially outward from a surface of the roll, the plurality of bonding points defining a pattern comprising a plurality of spaced apart arrays that extend in the cross and radial direction of the roll, and
thermally point bonding the web of fibers to form a nonwoven fabric in which a percent bonded surface area of the nonwoven fabric is less than about 12% and an average bond point packing value of the nonwoven fabric is from about 6.5 to 8 mm⁻¹.
